(19) United States
(12) Patent Application Publication
(10) Pub. No.: US 2005/0240100 A1
(43) Pub. Date: Oct. 27, 2005

(54) MRI imageable medical device

(76) Inventors: Xingwu Wang, Wellsville, NY (US); Howard J. Greenwald, Rochester, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 11/085,726

(22) Filed: Mar. 21, 2005

Related U.S. Application Data
(63) Continuation-in-part of application No. 10/887,521, filed on Jul. 7, 2004.
(63) Continuation-in-part of application No. 10/867,517, filed on Jun. 14, 2004.
(63) Continuation-in-part of application No. 10/810,916, filed on Mar. 26, 2004, now Pat. No. 6,846,985.
(63) Continuation-in-part of application No. 10/808,618, filed on Mar. 24, 2004.
(63) Continuation-in-part of application No. 10/786,198, filed on Feb. 25, 2004.
(63) Continuation-in-part of application No. 10/780,045, filed on Feb. 17, 2004.
(63) Continuation-in-part of application No. 10/747,472, filed on Dec. 29, 2003.
(63) Continuation-in-part of application No. 10/744,543, filed on Dec. 22, 2003.
(63) Continuation-in-part of application No. 10/442,420, filed on May 21, 2003, now Pat. No. 6,914,412.
(63) Continuation-in-part of application No. 10/409,505, filed on Apr. 8, 2003, now Pat. No. 6,815,609.

(51) Int. Cl. A61F 2/06

(52) U.S. Cl. 600/431; 623/1.46; 623/1.15; 174/35 R

(57) ABSTRACT

A coated assembly with an inductance of from about 0.1 to about 5 nanohenries and a capacitance of from about 0.1 to about 10 nanofarads. The coated assembly contains a stent and a coating. When the assembly is exposed to radio frequency electromagnetic radiation with a frequency of from 10 megahertz to about 200 megahertz, at least 90 percent of the electromagnetic radiation penetrates to the interior of the stent.

### CROSS-REFERENCE TO RELATED PATENT APPLICATION

This patent application is a continuation in part of each of applicants' copending patent application Ser. No. 10/887,521 (filed on Jul. 7, 2004), Ser. No. 10,867,517 (filed on Jun. 14, 2004), Ser. No. 10/810,916 (filed on Mar. 26, 2004), Ser. No. 10/808,618 (filed on Mar. 24, 2004), Ser. No. 10/786,198 (filed on Feb. 25, 2004), Ser. No. 10/780,045 (filed on Feb. 17, 2004), Ser. No. 10/747,472 (filed on Dec. 29, 2003), Ser. No. 10/744,543 (fled on Dec. 22, 2003), Ser. No. 10/442,420 (filed on May 21, 2003), and Ser. No. 10/409,505 (flied on Apr. 8, 2003). The entire disclosure of each of these patent applications is hereby incorporated by reference into this specification.

### FIELD OF THE INVENTION

A medical device comprised of a lumen that inhibits distortion of medical resonance images taken of the device. When the device is exposed to radio frequency electromagnetic radiation with a frequency of from 10 megahertz to about 200 megahertz, at least 90 percent of the radiofrequency electromagnetic radiation penetrates to the lumen of the device; and the radiation that penetrates to the lumen of the device is substantially uniform at different points within such interior.

### BACKGROUND OF THE INVENTION

Published U.S. patent application 2005/0033407 of Jan Weber et al. discusses vascular stents and discloses that “Vascular stents are known medical devices used in various vascular treatments of patients. Stents commonly include a tubular member that is moveable from a collapsed, low profile, delivery configuration to an expanded, deployed configuration. In the expanded configuration, an outer periphery of the stent frictionally engages an inner periphery of a lumen. The deployed stent then maintains the lumen such that it is substantially unoccluded and flow therethrough is substantially unrestricted. However, various stent designs substantially distort the surrounding of the stent during a Magnetic Resonance Imaging procedure” (see paragraph 0002). A similar teaching is contained in published United States patent application U.S. 2004/0093075 of Titus Kuehne As is disclosed in column 2 of this Kuehne patent application, “In the medical field, magnetic resonance imaging (MRI) is used to non-invasively produce medical information . . . . While researching heart problems, it was found that all the currently used metal stents distorted the magnetic resonance images of blood vessels. As a result, it was impossible to study the blood flow in the stents and the area directly around the stents for determining tissue response to different stents in the heart region.” (see paragraphs 0008 and 0009).

“Susceptibility artifacts,” “radiofrequency shielding,” and “in-stent restenosis” were discussed in an article by Elmar Spuentrup et al. entitled “Artifact-Free Coronary MRI Stents” that was published on Mar. 1, 2005 in Circulation, at pages 1019 to 1026; “Circulation” is available at http://www.circulationaha.org. As is disclosed at page 1019 of this Spuentrup et al. article, “Metallic stents are frequently used in the treatment of coronary artery stenosis; however, in-stent restenosis . . . is often observed. Although coronary magnetic resonance angiography (MRA) has been successfully implemented for visualization of the native proximal and middle portions of the coronary artery tree, the in-stent lumen cannot now be visualized because of susceptibility artifacts and radiofrequency shielding, resulting in a local signal void.”

There has been a substantial amount of speculation as to why “ . . . various stent designs substantially distort the surrounding of the stent during a Magnetic Resonance Imaging procedure;” and this phenomenon has been attributed to a “Faraday Cage effect.” Thus, and as is disclosed at lines 29-57 of Column 2 of U.S. Pat. No. 6,712,844 of Stephen Dirk Pacetti, “Because stents are constructed of electrically conductive materials, they suffer from a Faraday Cage effect when used with MRI's. Generically, a Faraday Cage is a box, cage, or array of electrically conductive material intended to shield its contents from electromagnetic radiation. The effectiveness of a Faraday Cage depends on the wave length of the radiation, the size of the mesh in the cage, the conductivity of the cage material, its thickness, and other variables. Stents do act as Faraday Cages in that they screen the stent lumen from the incident RF pulses of the MRI scanner. This prevents the proton spins of water molecules in the stent lumen from being flipped or excited. Consequently, the desired signal from the stent lumen is reduced by this diminution in excitation. Furthermore, the stent Faraday Cage likely impedes the escape of whatever signal is generated in the lumen. The stent's high magnetic susceptibility, however, perturbs the magnetic field in the vicinity of the implant. This alters the resonance condition of protons in the vicinity, thus leading to intravoxel dephasing with an attendant loss of signal. The net result with current metallic stents, most of which are stainless steel, is a signal void in the MRI images. Other metallic stents, such as those made from Nitinol, also have considerable signal loss in the stent lumen due to a combination of Faraday Cage and magnetic susceptibility effects.”

The contribution of a stent's ring structure to the “Faraday Cage effect” is also discussed in Pacetti's U.S. Pat. No. 6,712,844, wherein it is disclosed (at lines 10-31 of Column 3) that “Stents commonly have some form of ring elements. These are the portions of the stent that both expand and provide the radial strength. These ring elements are joined by links of various sorts. This combination of rings and links creates enclosed cells, and taken together, they create many continuous loops of metal. These loops can run around the circumference of the stent, or they can run in portions of the sent wall. Examination of any modern stent pattern will show a variety of hoops, rings, loops, or cells that provide many electrically conductive paths. It is this structure that creates a Faraday Cage, and its associated problems with MRI. Examples of such structures can be found in the Handbook of Coronary Stents, edited by Serruys and Kutryk . . . ”

The contribution of “ . . . ferromagnetic or electrically conductive materials . . . ” to the “Faraday Cage effect” is also discussed in U.S. Pat. No. 6,767,360 of Eckhard Alt, which discusses the problems involved with MRI imaging of stents. In column 2 of this patent, commencing at line 15, it is disclosed that “Magnetic resonance imaging (MRI) can be used to visualize internal features of the body if there is no magnetic resonance distortion. MRI has an excellent capability to visualize the vascular bed, with particularly accurate imaging of the vascular structure being feasible following the application of gadolinium, a contrast dye which enhances the magnetic properties of the blood and which stays within the vascular circulation . . . . Imaging procedures using MRI without need for contrast dye are emerging in the practice. But a current considerable factor weighing against the use of magnetic resonance imaging techniques to visualize implanted stents composed of ferromagnetic or electrically conductive materials is the inhibiting effect of such materials. These materials cause sufficient distortion of the magnetic resonance field to preclude imaging the interior of the stent. This effect is attributable to their Faradaic physical properties in relation to the electromagnetic energy applied during the MRI process.”

In the paragraph beginning at line 50 of column 2 of Alt's U.S. Pat. No. 6,767,360, reference was made to a “prior art” attempt to solve this imaging problem that was developed by Andreas Melzer et al. It is disclosed in this section of the patent that “In German application 197 46 735.0, which was filed as international patent application PCT/DE98/03045, published Apr. 22, 1999 as WO 99/19738, Melzer et al (Melzer, or the 99/19738 publication) disclose an MRI process for representing and determining the position of a stent, in which the stent has at least one passive oscillating circuit with an inductor and a capacitor. According to Melzer, the resonance frequency of this circuit substantially corresponds to the resonance frequency of the injected high-frequency radiation from the magnetic resonance system, so that in a locally limited area situated inside or around the stent, a modified signal answer is generated which is represented with spatial resolution. However, the Melzer solution lacks a suitable integration of an LC circuit within the stent.” The Alt patent does not specify in what respect(s) the “ . . . Melzer solution lacks a suitable integration of an LC circuit within the stent.”

One means of avoiding the “Faraday Cage effect” is to use stents made of nonconductive material. Thus, as is discussed in the paragraph beginning at line 54 of column 2 of Pacetti's U.S. Pat. No. 6,172,844, it is disclosed that “ . . . MRI . . . may become the standard diagnostic tool for heart disease. With these advances in imaging technologies, a stent that can be meaningfully imaged by MRI in an optimal manner would be advantageous. A non-metallic stent obviously solves the imaging problem. Metals, however, are the preferred material as they make strong, low profile stents possible. Unfortunately, most metal stents, particularly of stainless steel, obliterate MRI images of the anatomy in their vicinity and obscure the stent lumen in the image. By reducing the amount of metal in the stent, or by making the cells larger, or by having fewer cells, the Faraday Cage effect may be reduced. The RF radiation used in MRI has a wavelength of 2 to 35 meters depending on the scanner and environment of the stent. Therefore, the cell sizes of stents are already much smaller than the RF wavelength. Increasing the stent cell size would work only primarily by decreasing the amount of metal. This solution is limited by the need for stents to have adequate radial strength and scaffolding.”

To a similar effect is the teaching contained in paragraph 0007 of Jan Weber et al.'s published U.S. patent application 2005/0033407, wherein it is disclosed that “It is possible to build a stent out of polymer or other non-conducting materials such as ceramics. Building stents out of such non-conducting materials would avoid either of these MR artifacts. However, stents made form materials such as these would require larger strut dimensions to maintain adequate stent mechanical performance as compared to stents made out of metals.”

The problem with the prior art stents that have “adequate stent mechanical performance” is that magnetic resonance imaging is generally not able to view areas within such stents with adequate degrees of resolution. The desirability of being able to view areas within a stent is discussed in paragraph 0005 of Weber et al.'s published U.S. application 2005/0033407, wherein it is disclosed that “An ability to effectively view areas proximate a stent during an MRI procedure is desirable. In particular, viewing areas inside and proximate a tubular member of a stent may be desirable both during deployment and after deployment of the stent in a patient. However, various current stent designs prevent adequate imaging of the area surrounding the stent. Instead, the images are distorted and thus cannot be used.”

In paragraphs 0006 and 0007 of published U.S. patent application 2005/0033407, it was disclosed that, as of the filing date of such application (Aug. 7, 2003), none of the “current stent designs” had effectively solved the MRI imaging problem. It was disclosed that “The visibility of the inside of current stent designs during MRI procedures is blocked for two reasons. First of all, the permanent influence of the surrounding magnetic field by stents containing ferromagnetic materials prevents adequate imaging. A second reason that adequate imaging of the area inside the stent is blocked relates to induction currents (Eddy currents), induced in the closed cell metal stent structure due to the changes in the magnetic field generated by the MRI system during image sequencing. The result is that the MR visibility of the inside of the stent is shielded. It is possible to build a stent out of polymer or other non-conducting materials such as ceramics. Building stents out of such non-conducting materials would avoid either of these MR artifacts. However, stents made from materials such as these would require larger strut dimensions to maintain adequate stent mechanical performance as compared to stents made of metals.”

In paragraphs 31 and 32 of published U.S. patent application 2005/0033407, a discussion of the problems that are presented because of “Faraday's law” is presented. It is disclosed that “Another effect that commonly distorts the magnetic field around an intravascular device is associated with Faraday's Law. Faraday's Law simply states that any change in a magnetic environment of a coil will cause a voltage (emf) to be “induced” in the coil. Stent 150 can act as a coil when implanted in a subject during an MRI process. The change in magnetic environment is caused either by stent 150 moving or rotating within a nonuniform magnetic field, or by changes in the magnetic field proximate stent 150. For example, stent 150 may move due to the heart beating or magnetic field changes may be induced by gradient generator 130 or RF Source 140.”

In paragraph 32 of this published patent application, it is disclosed that “According to Faraday's Law, the induced emf in a coil is equal to the negative of the rate of change of magnetic flux through the coil times the number of turns in the coil. When an emf is generated by a change in magnetic flux, the polarity of the induced emf produces a current creating a magnetic field that opposes the change which produces it. Accordingly, the induced magnetic field inside any loop of wire acts to keep the magnetic flux inside the loop constant. In the case of a metallic stent, where each individual ring or cell, or combinations of cells, can act as a coil, the visibility within and around or adjacent the stent using an MRI can be blocked.”

In spite of all of the research reflected in the prior art, none of the prior art designs has provided a metallic stent that, when subjected to MRI imaging, provides adequate resolution of objects disposed within the stent.

It is an object of this invention to provide a stent assembly that, when it is exposed to MRI radiation, will allow at least 90 percent of this radiation to penetrate to the interior of the stent in a substantially uniform manner.

### SUMMARY OF THE INVENTION

In accordance with this invention, there is provided a medical device comprised of a lumen that inhibits distortion of medical resonance images taken of the device. When the device is exposed to radio frequency electromagnetic radiation with a frequency of from 10 megahertz to about 200 megahertz, at least 90 percent of such radio frequency electromagnetic radiation penetrates to the lumen of the device; and the concentration of the radio frequency electromagnetic radiation that penetrates to the lumen of the device is substantially identical at different points within such interior.

### BRIEF DESCRIPTION OF THE DRAWINGS

The above noted and other features of the invention will be better understood from the following drawings, and the accompanying description of them in the specification, wherein like numerals refer to like elements, and wherein:

FIG. 1 is a schematic diagram of one preferred seed assembly of the invention;

FIG. 1A is a schematic diagram of another preferred seed assembly of the invention;

FIG. 2 is a schematic illustration of one process of the invention that may be used to make nanomagnetic material;

FIG. 2A is a schematic illustration of a process that may be used to make and collect nanomagnetic particles;

FIG. 3 is a flow diagram of another process that may be used to make the nanomagnetic compositions of this invention;

FIG. 3A is a graph of the magnetic order of a nanomagnetic material plotted versus its temperature;

FIG. 4 is a phase diagram showing the phases in various nanomagnetic materials comprised of moieties A, B, and C;

FIGS. 4A and 4B illustrate how the magnetic order of the nanomagnetic particles of this invention is destroyed at a temperature in excess of the phase transition temperature;

FIG. 5 is a schematic representation of what occurs when an electromagnetic field is contacted with a nanomagnetic material;

FIG. 5A illustrates the coherence length of the nanomagnetic particles of this invention;

FIG. 6 is a schematic sectional view of a shielded conductor assembly that is comprised of a conductor and, disposed around such conductor, a film of nanomagnetic material;

FIGS. 7A through 7E are schematic representations of other shielded conductor assemblies that are similar to the assembly of FIG. 6;

FIG. 8 is a schematic representation of a deposition system for the preparation of aluminum nitride materials;

FIG. 9 is a schematic, partial sectional illustration of a coated substrate that, in the preferred embodiment illustrated, is comprised of a coating disposed upon a stent;

FIG. 9A is a schematic illustration of a coated substrate that is similar to the coated substrate of FIG. 9 but differs therefrom in that it contains two layers of dielectric material;

FIG. 10 is a schematic view of a typical stent that is comprised of wire mesh constructed in such a manner as to define a multiplicity of openings;

FIG. 11 is a graph of the magnetization of an object (such as an uncoated stent, or a coated stent) when subjected to an electromagnetic filed, such as an MRI field;

FIG. 11A is a graph of the magnetization of a composition comprised of species with different magnetic susceptibilities when subjected to an electromagnetic field, such as an MRI field;

FIG. 12 is a graph of the reactance of an object (such as an uncoated stent, or a coated stent) when subjected to an electromagnetic filed, such as an MRI field;

FIG. 13 is a graph of the image clarity of an object (such as an uncoated stent, or a coated stent) when subjected to an electromagnetic filed, such as an MRI field;

FIG. 14 is a phase diagram of a material that is comprised of moieties A, B, and C;

FIG. 15 is a schematic view of a coated substrate comprised of a substrate and a multiplicity of nanoelectrical particles;

FIGS. 16A and 16B illustrate the morphological density and the surface roughness of a coating on a substrate;

FIG. 17A is a schematic representation of a stent comprised of plaque disposed inside the inside wall;

FIG. 17B illustrates three images produced from the imaging of the stent of FIG. 17A, depending upon the orientation of such stent in relation to the MRI imaging apparatus reference line;

FIG. 17C illustrates three images obtained from the imaging of the stent of FIG. 17A when the stent has the nanomagnetic coating of this invention disposed about it;

FIGS. 18A and 18B illustrate a hydrophobic coating and a hydrophilic coating, respectively, that may be produced by the process of this invention;

FIG. 19 illustrates a coating disposed on a substrate in which the particles in their coating have diffused into the substrate to form a interfacial diffusion layer;

FIG. 20 is a sectional schematic view of a coated substrate comprised of a substrate and, bonded thereto, a layer of nano-sized particles;

FIG. 20A is a partial sectional view of an indentation within a coating that, in turn, is coated with a multiplicity of receptors;

FIG. 20B is a schematic of an electromagnetic coil set aligned to an axis and which in combination create a magnetic standing wave;

FIG. 20C is a three-dimensional schematic showing the use of three sets of magnetic coils arranged orthogonally;

FIG. 21 is a schematic illustration of one process for preparing a coating with morphological indentations;

FIG. 22 is a schematic illustration of a drug molecule disposed inside of a indentation;

FIG. 23 is a schematic illustration of one preferred process for administering a drug into the arm of a patient near a stent via an injector;

FIG. 24 is a schematic illustration of a preferred binding process of the invention;

FIG. 25 is a schematic view of a preferred coated stent of the invention;

FIG. 26 is a graph of a typical response of a magnetic drug particle to an applied electromagnetic field;

FIGS. 27A and 27B illustrate the effect of applied fields upon a nanomagnetic and upon magnetic drug particles;

FIG. 28 is graph of a preferred nanomagnetic material and its response to an applied electromagnetic field, in which the applied field is applied against the magnetic moment of the nanomagnetic material;

FIG. 29 illustrates the forces acting upon a magnetic drug particle as it approaches nanomagnetic material;

FIG. 30 illustrates the situation that occurs after the drug particles have migrated into the layer of polymeric material and when one desires to release such drug particles;

FIG. 31 illustrates the situation that occurs after the drug particles have migrated into the layer of polymeric material but when no external electromagnetic field is imposed:

FIG. 32 is a partial view of a coated container over which is disposed a layer 5002 of material which changes its dimensions in response to an applied magnetic field;

FIG. 33 is a partial view of magnetostrictive magnetostrictive material prior to the time an orifice has been created in it;

FIG. 34 is a schematic illustration of a magnetostrictive material bounded by nanomagnetic material;

FIG. 35 is a schematic illustration of a preferred implantable device of this invention with improved MRI imageability;

FIG. 36 is a sectional view of a component of a preferred stent assembly;

FIG. 37 is a graph of the relative permeability of a coating of nanomagnetic material, and a coating of ferrite material, over the range from 0 hertz to greater than 1 gigahertz;

FIG. 38 is a schematic illustration of the effects on the deposition of iron onto a substrate of a magnetron, illustrating how the concentration of iron decreases as the coated film thickness increases;

FIG. 39 is a graph of the concentration of iron in the coating depicted in FIG. 38 versus the thickness of the coating;

FIG. 40 is a schematic of a preferred process for imaging a coated stent; and

FIG. 41 is a schematic illustration of the resolution obtained with applicants' coated stent and, in particular, of the resolution obtained by MRI imaging of objects disposed within such coated stent;

FIG. 42 is a flow diagram of a preferred phase imaging process;

FIG. 43 is a schematic illustration of the phase shift obtained with applicants' coated stent; and

FIG. 44 is a schematic illustration of one preferred coated stent assembly;

FIG. 45 is a sectional view of a preferred coated ring assembly;

FIG. 46 is a sectional view of another coated ring assembly;

FIG. 47 is a sectional view of yet another coated ring assembly;

FIG. 48 is a sectional view of yet another coated ring assembly;

FIG. 49 is a schematic illustration of the effect of MRI radiation upon in-stent restenosis of a prior art stent;

FIG. 50 is a schematic illustration of the effect of MRI radiation upon in-stent restenosis of a preferred stent of this invention;

FIG. 51 is a schematic of the bandwidth of one preferred coated stent of the invention; and

FIGS. 52 through 55 are schematic illustrations of some preferred coated substrates that provide the desired passive resonance properties for imaging in-stent restenosis.

### DESCRIPTION OF THE PREFERRED EMBODIMENT

In the first part of this specification, certain assemblies that contain nanomagnetic material, and/or certain processes for making nanomagnetic material, will be briefly described. Thereafter, in the second part of this specification, an improved stent assembly whose lumen is readily imageable under magnetic resonance imaging conditions will be described.

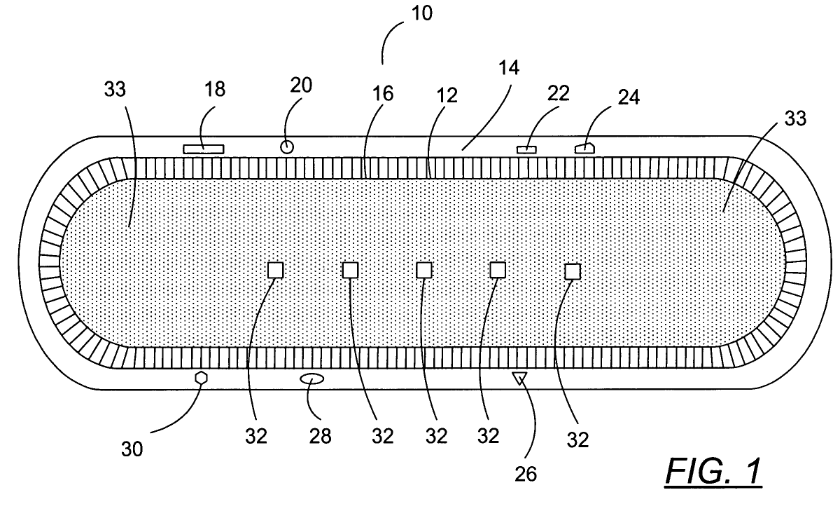

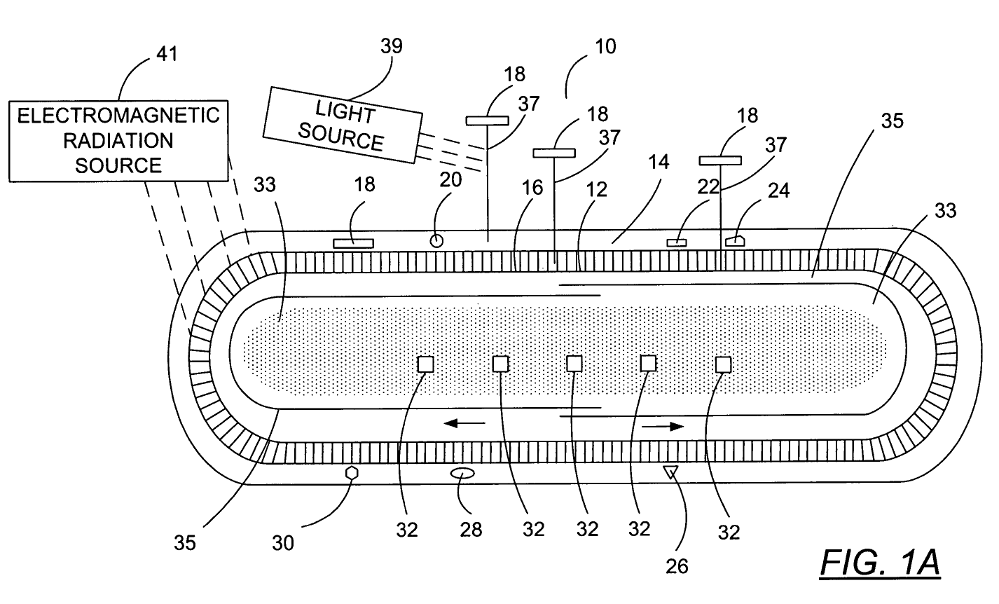

FIG. 1 is a schematic diagram of a preferred seed assembly 10 of this invention that may, in one preferred embodiment, contain nanomagnetic material. The FIGS. 1 and 1A of this specification are substantially identical to the FIGS. 1 and 1A of published United States patent application U.S. 2005/0025797, published on Feb. 5, 2005, the entire disclosure of which is hereby incorporated by reference into this specification; in particular, and without limitation, the disclosure of pages 2 through 40 of such published patent application, are hereby incorporated by reference into this specification.

Referring again to FIGS. 1 and 1A, the seed assembly 10 is preferably comprised of a polymeric material 14 disposed above the sealed container 12. In the embodiment depicted in FIG. 1, the polymeric material 14 is contiguous with a layer 16 of magnetic material. In another embodiment, not shown in FIG. 1, the polymeric material 14 is contiguous with the sealed container 12.

In one embodiment, depicted in FIG. 1A, a photosensitive linker 37 is bound to layer 16 comprised of nanomagnetic material. In yet another embodiment, the photosensitive linker 37 is bound to the surface of container 12.

Referring again to FIGS. 1 and 1A, the sealed container 12 is comprised of one or more nanomagnetic particles 32. Furthermore, in the preferred embodiment depicted in FIGS. 1 and 1A, a film 16 is disposed around sealed container 12, and this film is also preferably comprised of nanomagnetic particles 32 (not shown for the sake of simplicity of representation).

In one embodiment, and disposed within sealed container 12, there is collection of nanomagnetic particles 32 with an average particle size of less than about 100 nanometers. The average coherence length between adjacent nanomagnetic particles is preferably less than about 100 nanometers. Some similar nanomagnetic particles are disclosed in applicants' U.S. Pat. No. 6,502,972, the entire disclosure of which is hereby incorporated by reference into this specification.

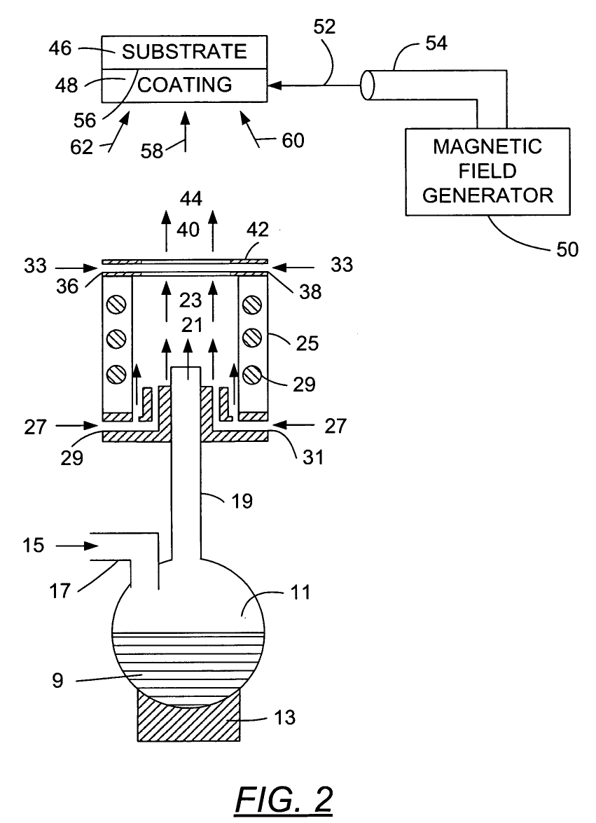

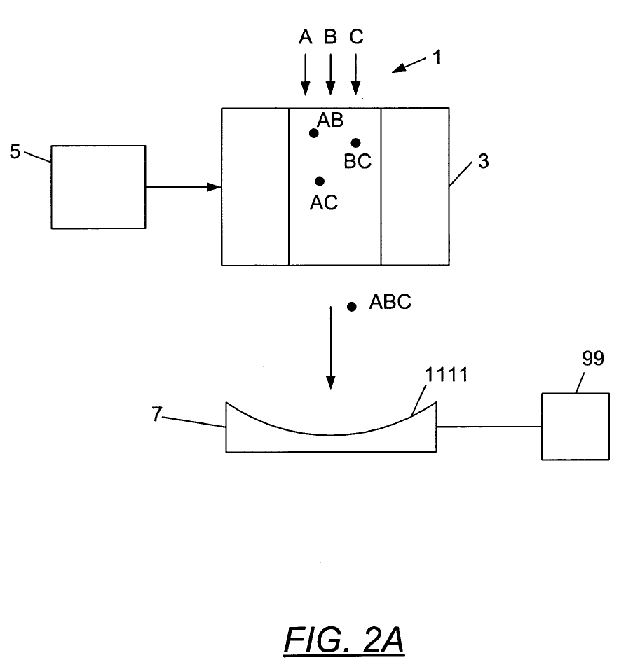

FIG. 2 is a schematic illustration of one process of the invention that may be used to make nanomagnetic material. This FIG. 2 is similar in many respects to the FIG. 1 of U.S. Pat. No. 5,213,851, the entire disclosure of which is hereby incorporated by reference into this specification.

Referring to FIG. 2, it is preferred that the reagents charged into misting chamber 11 will be sufficient to form a nano-sized ferrite in the process. The term ferrite, as used in this specification, refers to a material that exhibits ferromagnetism. Ferrites are extensively described in U.S. Pat. No. 5,213,851, the entire disclosure of which is hereby incorporated by reference into this specification.

FIG. 2 of published U.S. patent application 2005/0025797 A1 is substantially identical to the FIG. 2 of this case; and pages 41-46 of such published patent application describe its FIG. 2. The entire disclosure of such pages 41-46 is hereby incorporated by reference into this specification.

Referring again to FIG. 2, the solution 9 will preferably comprise reagents necessary to form the required magnetic material. For example, in one embodiment, in order to form the spinel nickel ferrite of the formula NiFe₂O₄, the solution should contain nickel and iron, which may be present in the form of nickel nitrate and iron nitrate.

In one embodiment, illustrated in FIG. 2A, the substrate is cooled so that nanomagnetic particles are collected on such substrate. Referring to FIG. 2A, a precursor 1 that preferably contains moieties A, B, and C (which are described elsewhere in this specification) are charged to reactor 3; the reactor 3 may be the plasma reactor depicted in FIG. 2, and/or it may be the sputtering reactor described elsewhere in this specification.

Referring again to FIG. 2A, an energy source 5 is preferably used in order to cause reaction between moieties A, B, and C. The energy source 5 may be an electromagnetic energy source that supplies energy to the reactor 3. Within reactor 3 moieties A, B, and C are preferably combined into a metastable state. This metastable state is then caused to travel towards collector 7. Prior to the time it reaches the collector 7, the ABC moiety is formed, either in the reactor 3 and/or between the reactor 3 and the collector 7.

In one embodiment, collector 7 is preferably cooled with a chiller 99 so that its surface 111 is at a temperature below the temperature at which the ABC moiety interacts with surface 111; the goal is to prevent bonding between the ABC moiety and the surface 111. In one embodiment, the surface 111 is at a temperature of less than about 30 degrees Celsius. In another embodiment, the temperature of surface 111 is at the liquid nitrogen temperature, i.e., about 77 degrees Kelvin.

After the ABC moieties have been collected by collector 7, they are removed from surface 111.

The substrate 46 may be moved in a plane that is substantially parallel to the top of plasma chamber 25. Alternatively, it may be moved in a plane that is substantially perpendicular to the top of plasma chamber 25. In one embodiment, the substrate 46 is moved stepwise along a predetermined path to coat the substrate only at certain predetermined areas.

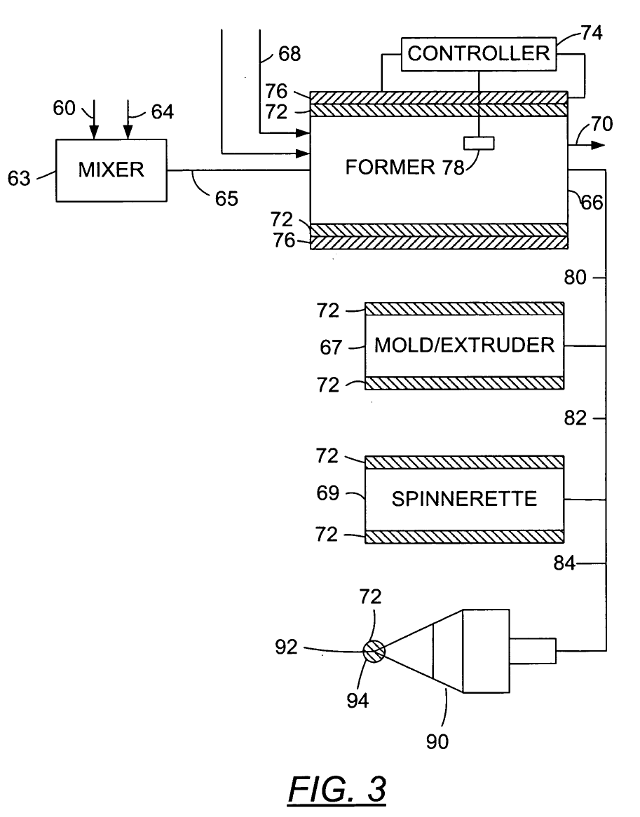

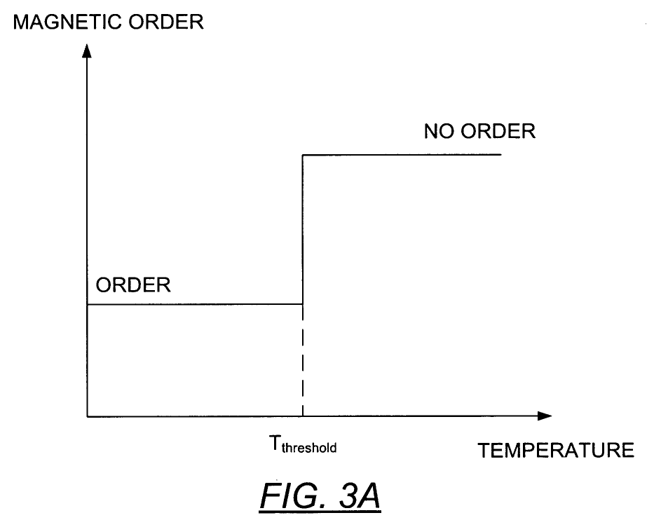

FIG. 3 is a flow diagram of another process that may be used to make the nanomagnetic compositions of this invention. This FIG. 3 is substantially identical to the FIG. 3 of published U.S. patent application 2005/0025797 A1, published on Feb. 3, 2005; pages 46-49 of such published patent application describe such FIG. 3; and the entire disclosure of such pages 46-49 of such published U.S. patent application is hereby incorporated by reference into this specification.

Referring to FIG. 3 of the instant case, nano-sized ferromagnetic material(s), with a particle size less than about 100 nanometers are charged via line 60 to mixer 62. It is preferred to charge a sufficient amount of such nano-sized material(s) so that at least about 10 weight percent of the mixture formed in mixer 62 is comprised of such nano-sized material. In one embodiment, at least about 40 weight percent of such mixture in mixer 62 is comprised of such nano-sized material. In another embodiment, at least about 50 weight percent of such mixture in mixer 62 is comprised of such nano-sized material. In one embodiment, one or more binder materials are charged via line 64 to mixer 62.

Referring again to FIG. 3, the mixture within mixer 62 is preferably stirred until a substantially homogeneous mixture is formed. Thereafter, it may be discharged via line 65 to former 66.

In the embodiment depicted, former 66 is also preferably comprised of an electromagnetic coil 72 that, in response from signals from controller 74, can control the extent to which a magnetic field is applied to the mixture within the former 66 (and also within the mold 67 and/or the spinnerette 69).

In the embodiment depicted in FIG. 3, a sensor 78 preferably determines the extent to which the desired nanomagnetic properties have been formed with the nano-sized material in the former 66; and, as appropriate, the sensor 78 imposes a magnetic field upon the mixture within the former 66 until the desired properties have been obtained.

When the mixture within former 66 has the desired combination of properties and/or prior to that time, some or all of such mixture may be discharged via line 80 to a mold/extruder 67 wherein the mixture can be molded or extruded into a desired shape. A magnetic coil 72 also preferably may be used in mold/extruder 67 to help align the nano-sized particles.

Alternatively, some or all of the mixture within former 66 may be discharged via line 82 to a spinnerette 69, wherein it may be formed into a fiber (not shown).

Nanomagnetic Compositions comprised of Moieties A, B, and C

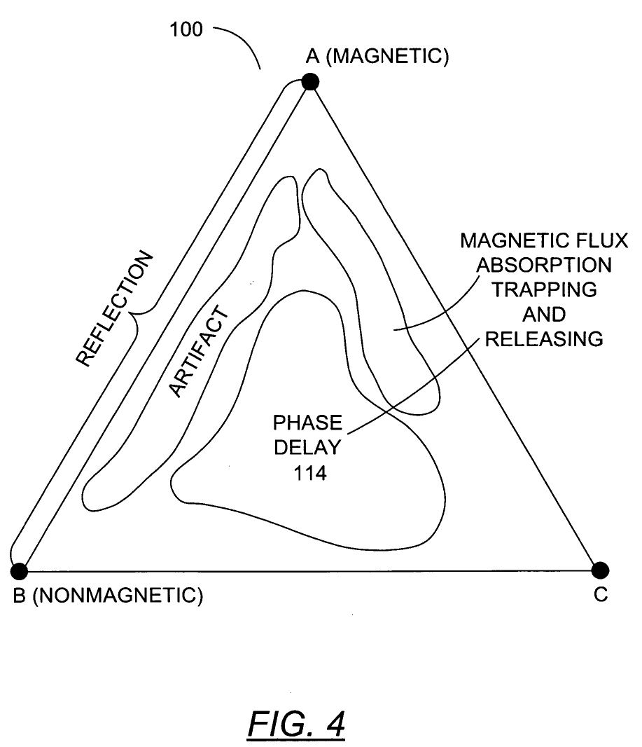

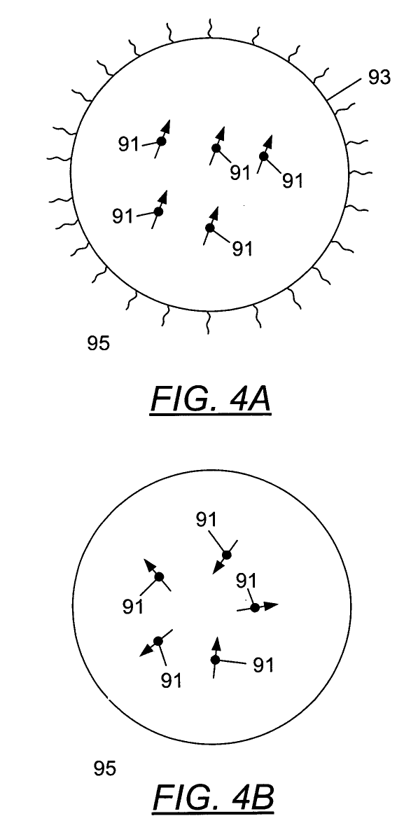

The aforementioned process described in the preceding section of this specification, and the other processes described in this specification, may each be adapted to produce other, comparable nanomagnetic structures, as is illustrated in FIG. 4. This FIG. 4 is substantially identical to the FIG. 4 of published United States patent application U.S. 2005/0025797 A1, published on Feb. 3, 2005, the entire disclosure of which is hereby incorporated by reference into this specification. In particular, and without limitation, pages 49-50 of such published United States patent application are hereby incorporated by reference into this specification.

Referring to FIG. 4 of the instant case, and in the preferred embodiment depicted therein, a phase diagram 100 is presented. As is illustrated by this phase diagram 100, the nanomagnetic material used in this embodiment of the invention preferably is comprised of one or more of moieties A, B, and C.

In the embodiment depicted, the moiety A depicted in phase diagram 100 is preferably comprised of a magnetic element selected from the group consisting of a transition series metal, a rare earth series metal, or actinide metal, a mixture thereof, and/or an alloy thereof. In one embodiment, the moiety A is iron. In another embodiment, moiety A is nickel. In yet another embodiment, moiety A is cobalt. In yet another embodiment, moiety A is gadolinium. elements.

In one preferred embodiment, two or more A moieties are present, as atoms. In one aspect of this embodiment, the magnetic susceptibilities of the atoms so present are both positive.

In one embodiment, two or more A moieties are present, at least one of which is iron. In one aspect of this embodiment, both iron and cobalt atoms are present.

When both iron and cobalt are present, it is preferred that from about 10 to about 90 mole percent of iron be present by mole percent of total moles of iron and cobalt present in the ABC moiety. In another embodiment, from about 50 to about 90 mole percent of iron is present. In yet another embodiment, from about 60 to about 90 mole percent of iron is present. In yet another embodiment, from about 70 to about 90 mole percent of iron is present.

The transition series metals include chromium, manganese, iron, cobalt, and nickel; and one or more of them (and/or their alloys) may be used as the moiety A. One may use alloys of iron, cobalt and nickel such as, e.g., iron-aluminum, iron-carbon, iron-chromium, iron-cobalt, iron-nickel, iron nitride (Fe₃N), iron phosphide, iron-silicon, iron-vanadium, nickel-cobalt, nickel-copper, and the like. One may use alloys of manganese such as, e.g., manganese-aluminum, manganese-bismuth, MnAs, MnSb, MnTe, manganese-copper, manganese-gold, manganese-nickel, manganese-sulfur and related compounds, manganese-antimony, manganese-tin, manganese-zinc, Heusler alloy W, and the like. One may use compounds and alloys of the iron group, including oxides of the iron group, halides of the iron group, borides of the transition elements, sulfides of the iron group, platinum and palladium with the iron group, chromium compounds, and the like.

One may use a rare earth and/or actinide metal such as, e.g., Ce, Pr, Nd, Pm, Sm, Eu, Gd, Th, Dy, Ho, Er, Tm, Yb, Lu, La, mixtures thereof, and alloys thereof. One may also use one or more of the actinides such as, e.g., the actinides of Th, Pa, U, Np, Pu, Am, Cm, Bk, Cf, Es, Fm, Md, No, Lr, Ac, and the like.

In one preferred embodiment, illustrated in FIG. 4, moiety A is selected from the group consisting of iron, nickel, cobalt, alloys thereof, and mixtures thereof. In this embodiment, the moiety A is magnetic, i.e., it has a relative magnetic permeability of from about 1 to about 500,000. As is known to those skilled in the art, relative magnetic permeability is a factor, being a characteristic of a material, which is proportional to the magnetic induction produced in a material divided by the magnetic field strength; it is a tensor when these quantities are not parallel. See, e.g., page 4-128 of E. U. Condon et al.'s “Handbook of Physics” (McGraw-Hill Book Company, Inc., New York, N.Y., 1958). The relative alternating current magnetic permeability is the relative magnetic permeability the material exhibits in the presence of an alternating current electromagnetic field.

In one preferred embodiment, the A moiety has a relative magnetic permeability of from about 1 to about 20,000.

The moiety A of FIG. 4 also preferably has a saturation magnetization of from about 1 to about 36,000 Gauss, and a coercive force of from about 0.01 to about 5,000 Oersteds. In one embodiment, the A moiety has a saturation magnetization of at least about 1,000 electromagnetic units per cubic centimeter and, more preferably, at least about 1,500 electromagnetic units per cubic centimeter. In one aspect of this preferred embodiment, the A moiety has a coercive force of less than about 100 Oersteds.

The moiety A of FIG. 4 may be present in the nanomagnetic material either in its elemental form, as an alloy, in a solid solution, or as a compound.

It is preferred at least about 1 mole percent of moiety A be present in the nanomagnetic material (by total moles of A, B, and C), and it is more preferred that at least 10 mole percent of such moiety A be present in the nanomagnetic material (by total moles of A, B, and C). In one embodiment, at least 60 mole percent of such moiety A is present in the nanomagnetic material, (by total moles of A, B, and C.)

In terms of the weight percent concentration of the A moiety in the nanomagnetic material, it is preferred that such nanomagnetic material comprise from about 1 to about 20 weight percent of the A moiety and, more preferably, from about 5 to about 20 weight percent. In another embodiment, the A moiety is present in the “ABC material” at a concentration of from 9 to about 15 weight percent.

In one embodiment, the nanomagnetic material has the formula A₁A₂(B)ₓC₁(C₂)_{y}, wherein each of A₁ and A₂ are separate magnetic A moieties, as described above; B is as defined elsewhere in this specification; x is an integer from 0 to 1; each of C₁ and C₂ is as descried elsewhere in this specification and is a separate C moiety; and y is an integer from 0 to 1.

In this embodiment, there are always two distinct A moieties, such as, e.g., nickel and iron, iron and cobalt, etc. The A moieties may be present in equimolar amounts; or they may be present in non-equimolar amount.

In one preferred embodiment, the A moiety consists of or comprises one or more isotopes of cobalt. In one aspect of this embodiment, both iron and cobalt are present as the composite A moiety with from about 0.01 to about 100 parts of cobalt being used for each part of iron.

In one aspect of this embodiment, either or both of the A₁ and A₂ moieties are radioactive.

Referring again to FIG. 4, and to the preferred embodiment depicted therein, in this embodiment, there may be, but need not be, a B moiety (such as, e.g., aluminum). There preferably are at least two C moieties such as, e.g., oxygen and/or nitrogen; carbon may also be present as a C moiety. The A moieties, in combination, comprise at least about 80 mole percent of such a composition; and they preferably comprise at least 90 mole percent of such composition.

In one preferred embodiment, the B and C moieties, in combination, represent from about 80 to about 99 weight percent of the combined weight of the ABC composition. Without wishing to be bound to any particular theory, applicants believe that the B and C moieties may combine to form a dielectric matrix within which the A moiety is disposed, wherein said dielectric matrix has a relative dielectric constant of from between 1 to 2000.

In one embodiment, composite ABC moiety preferably has a conductivity of from about 10⁻¹³ (ohm-meter)⁻¹ to about 10⁸ (ohm-meter)⁻¹ and, more preferably, from about 10⁻³ (ohm-meter)⁻¹ to about 10 (ohm-meter)⁻¹.

In one aspect of this embodiment, when the ABC moiety is disposed as a coating with a thickness of 1 micron on a substrate (such as a stent), the conductivity along its cross-section will vary due to a gradient in the concentration of the A moiety and/or the C moiety, both of which gradients are described elsewhere in this specification. The conductivity from the top to the bottom of such a coating will generally vary from about 10⁻¹³ (ohm-meter)⁻¹ to about 10⁻³ (ohm-meter)⁻¹.

However, the conductivity will be greater in those portions of the coating that contain more of the A moiety.

Without wishing to be bound to any particular theory, applicants believe that the individual combinations of A moieties disposed in BC matrices form local resonant circuits that facilitate the transfer of radio frequency energy into and out of objects on which the nanomagnetic material is disposed.

When two C moieties are present, and when the two C moieties are oxygen and nitrogen, they preferably are present in a mole ratio such that from about 10 to about 90 mole percent of oxygen is present, by total moles of oxygen and nitrogen. It is preferred that at least about 60 mole percent of oxygen be present. In one embodiment, at least about 70 mole percent of oxygen is so present. In yet another embodiment, at least 80 mole percent of oxygen is so present.

In one preferred embodiment, at least two C moieties are present, and these two C moieties are oxygen and nitrogen. In this embodiment, the mole ratio of oxygen to nitrogen in the coating is preferably from 1/10 to 10/1 and, more preferably, from about 1/5 to about 5/1.

In one preferred embodiment, at least one of the C moieties is carbon, and at least another of the C moieties is oxygen. In this embodiment, nitrogen may also be present as a third C moiety.

One may measure the surface coating comprising the nanomagnetic material, measuring the first 8.5 nanometers of material. When such surface is measured, it is preferred that at least 50 mole percent of oxygen, by total moles of oxygen and nitrogen, be present in such surface. It is preferred that at least about 60 mole percent of oxygen be present. In one embodiment, at least about 70 mole percent of oxygen is so present. In yet another embodiment, at least 80 mole percent of oxygen is so present.

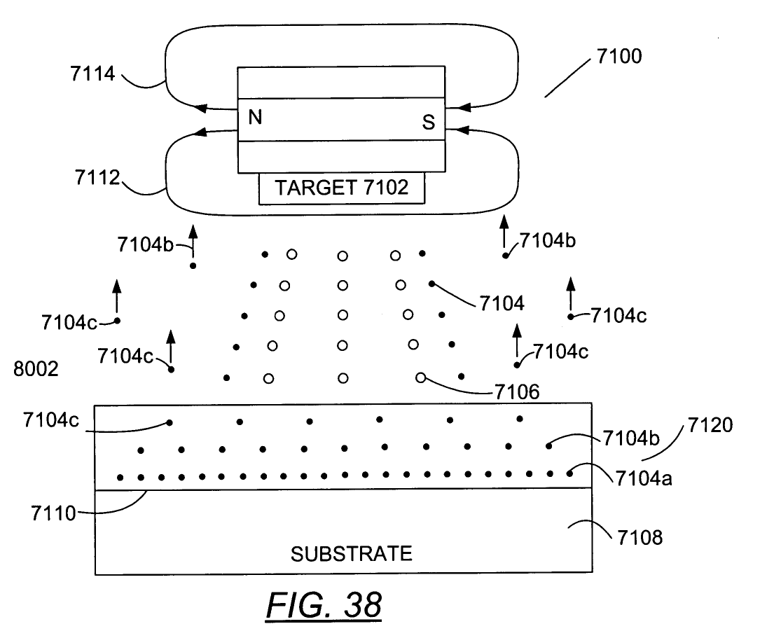

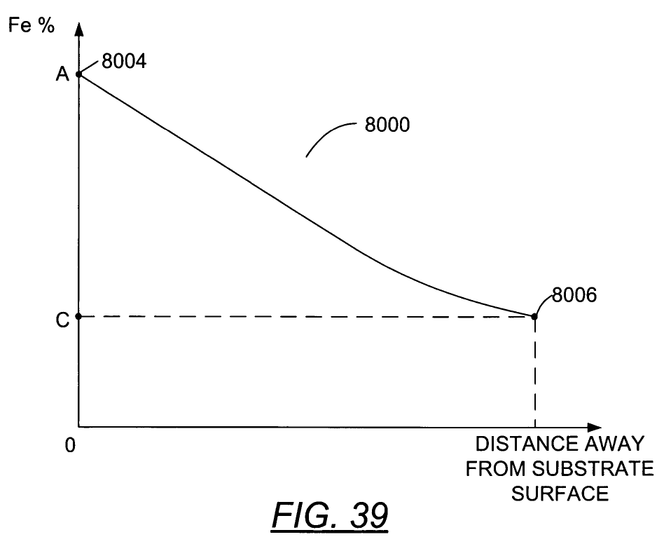

By comparison, and in one preferred embodiment (see FIGS. 38 and 39), in the “bottom half” of the nanomagnetic coating (i.e., that portion of the coating that is connected to the substrate), more than 1.5 times as much of the “A moiety” appears as does in the “top half” (i.e., that portion of the coating closest to the sputtering machine). Without wishing to be bound to any particular theory, applicants believe that this differential in the concentration of the A moiety in the coating is caused by the attraction of the A moiety to both the surface of the substrate, and to the magnetron used in sputtering. The more than a film is deposited upon a coating, and the further away that the sputtered particles are from the surface of the substrate, the less attraction surface has for the sputtered particles, and the more such sputtered particles are attracted backward towards the magnetron. Consequently, the closer the coating is to the surface of the substrate, the greater its concentration of A moiety or moieties.

Without wishing to be bound to any particular theory, applicants believe that the presence of two distinct A moieties in their composition, and/or two distinct C moieties (such as, e.g., oxygen and nitrogen), provides better magnetic properties for applicants' nanomagnetic materials.

In the embodiment depicted in FIG. 4, in addition to moiety A, it is preferred to have moiety B be present in the nanomagnetic material. In this embodiment, moieties A and B are admixed with each other. The mixture may be a physical mixture, it may be a solid solution, it may be comprised of an alloy of the A/B moieties, etc.

The Squareness of the Nanomagnetic Particles of the Invention

As is known to those skilled in the art, the squareness of a magnetic material is the ratio of the residual magnetic flux and the saturation magnetic flux density. Reference may be had, e.g., to U.S. Pat. Nos. 6,627,313, 6,517,934, 6,458,452, 6,391,450, 6,350,505, 6,248,437, 6,194,058, 6,042,937, 5,998,048, 5,645,652, and the like. The entire disclosure of each of such United States patents is hereby incorporated by reference into this specification.

In one embodiment, the squareness of applicants' nanomagnetic material 32 is from about 0.05 to about 1.0. In one aspect of this embodiment, such squareness is from about 0.1 to about 0.9. In another aspect of this embodiment, the squareness is from about 0.2 to about 0.8. In applications where a large residual magnetic moment is desired, the squareness is preferably at least about 0.8.

Referring again to FIG. 4, and in the preferred embodiment depicted therein, the nanomagnetic material may be comprised of 100 percent of moiety A, provided that such moiety A has the required normalized magnetic interaction (M). Alternatively, the nanomagnetic material may be comprised of both moiety A and moiety B. In one embodiment, the A moieties comprise at least about 80 mole percent (and preferably at least about 90 mole percent) of the total moles of the A, B, and C moieties.

When moiety B is present in the nanomagnetic material, in whatever form or forms it is present, it is preferred that it be present at a mole ratio (by total moles of A and B) of from about 1 to about 99 percent and, preferably, from about 10 to about 90 percent.

The B moiety, in one embodiment, in whatever form it is present, is preferably nonmagnetic, i.e., it has a relative magnetic permeability of about 1.0. One may use, e.g., such elements as silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, and the like.

In one embodiment, the B moiety has a relative magnetic permeability that is about equal to 1 plus the magnetic susceptibility. The relative magnetic susceptibilities of silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, copper, cesium, cerium, hafnium, iodine, iridium, lanthanum, lithium, lutetium, manganese, molybdenum, potassium, sodium, strontium, praseodymium, rhenium, rhodium, rubidium, ruthenium, scandium, selenium, tantalum, technetium, tellurium, chromium, thallium, thorium, thulium, titanium, vanadium, zinc, yttrium, ytterbium, zirconium, and the like. Reference may be had, e.g., to pages E-118 through E 123 of the aforementioned CRC Handbook of Chemistry and Physics.

In one preferred embodiment, the B moiety is titanium, and it is present in combination with both oxygen and nitrogen to form BC compositions such as titanium oxide, titanium nitride.

In another embodiment, the B moiety is barium and titanium, whereby barium titanate, and/or barium titanium nitride materials may be formed in the presence of C moieties such as oxygen and/or nitrogen.

In one embodiment, the nanomagnetic particles may be represented by the formula AₓB_{y}C_{z} wherein x+y+z is equal to 1. In this embodiment the ratio of x/y is at least 0.1 and preferably at least 0.2; and the ratio of z/x is from 0.001 to about 0.5.

In one preferred embodiment, the B material is aluminum and the C material is nitrogen, whereby an AlN moiety is formed. Applicants believe that aluminum nitride (and comparable materials) are both electrically insulating and thermally conductive, thus providing a excellent combination of properties for certain end uses.

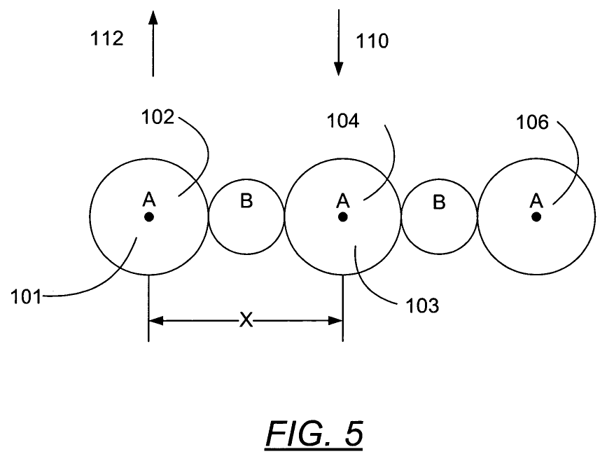

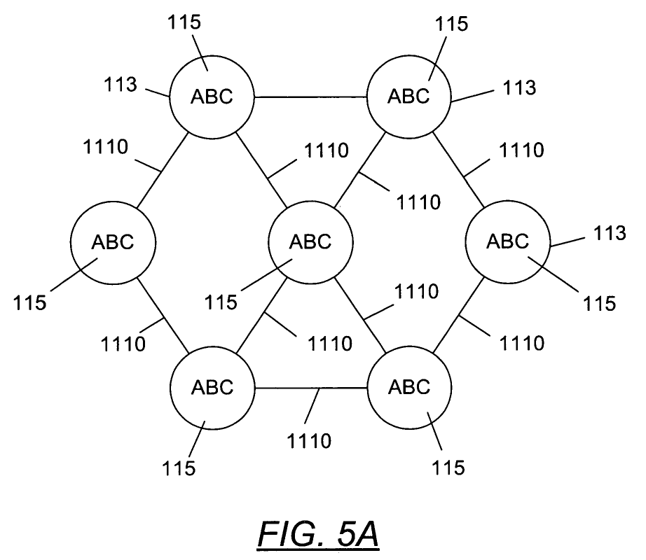

Referring again to FIGS. 4 and 5, when an electromagnetic field 110 is incident upon the nanomagnetic material comprised of A and B (see FIG. 4), such a field will be reflected to some degree depending, e.g., upon the ratio of moiety A and moiety B. In one embodiment, it is preferred that at least 1 percent of such field is reflected in the direction of arrow 112 (see FIG. 5). In another embodiment, it is preferred that at least about 10 percent of such field is reflected. In yet another embodiment, at least about 90 percent of such field is reflected. Without wishing to be bound to any particular theory, applicants believe that the degree of reflection depends upon the concentration of A in the A/B mixture.

Referring again to FIG. 4, and in one embodiment, the nanomagnetic material is comprised of moiety A, moiety C, and optionally moiety B. The moiety C is preferably selected from the group consisting of elemental oxygen, elemental nitrogen, elemental carbon, elemental fluorine, elemental chlorine, elemental hydrogen, and elemental helium, elemental neon, elemental argon, elemental krypton, elemental xenon, elemental fluorine, elemental sulfur, elemental hydrogen, elemental helium, the elemental chlorine, elemental bromine, elemental iodine, elemental boron, elemental phosphorus, and the like. In one aspect of this embodiment, the C moiety is selected from the group consisting of elemental oxygen, elemental nitrogen, and mixtures thereof.

In one embodiment, the C moiety is chosen from the group consisting of oxygen, nitrogen, and mixtures thereof. In one aspect of this embodiment, the C moiety is a mixture of oxygen and nitrogen, wherein the oxygen is present at a concentration from about 10 to about 90 mole percent, by total moles of oxygen and nitrogen.

Referring again to FIG. 4, the area 114 produces a composition which optimizes the degree to which magnetic flux are initially trapped and/or thereafter released by the composition when a magnetic field is withdrawing from the composition.

Thus, and referring again to FIG. 4, one may optimize the A/B/C composition to preferably be within the area 114. In general, the A/B/C composition has molar ratios such that the ratio of A/(A and C) is from about 1 to about 99 mole percent and, preferably, from about 10 to about 90 mole percent. In one preferred embodiment, such ratio is from about 40 to about 60 molar percent.

The molar ratio of A/(A and B and C) generally is from about 1 to about 99 molar percent and, preferably, from about 10 to about 90 molar percent. In one embodiment, such molar ratio is from about 30 to about 60 molar percent.

The molar ratio of B/(A plus B plus C) generally is from about 1 to about 99 mole percent and, preferably, from about 10 to about 40 mole percent.

The molar ratio of C/(A plus B plus C) generally is from about 1 to about 99 mole percent and, preferably, from about 10 to about 50 mole percent.

In one embodiment, the composition of the nanomagnetic material is chosen so that the applied electromagnetic field 110 is absorbed by the nanomagnetic material by less than about 1 percent; thus, in this embodiment, the applied magnetic field 110 is substantially restored by correcting the time delay.

The nanomagnetic material of this invention is comprised of nano-sized particles. As used herein, the nano-sized particle describes a physical moiety whose maximum dimension is less than 100 nanometers. Without wishing to be bound to any particular theory, applicants believe that the nanomagnetic particles in their material comprise at least the aforementioned A moiety.

By way of illustration and not limitation, the nanomagnetic particles may be in the form of crystallites with a length of from about 3 to about 30 nanometers and width of from about 1 to about 5 nanometers. In one embodiment, the nanomagnetic particles preferably have an aspect ratio of at least about 1.1 and, more preferably, from about 1.2 to about 10. In this embodiment, and without limitation, it is preferred that this crystallite materials be superparamagnetic.

As is known to those skilled in the art, superparamagnetic materials generally have a relatively low magnetic properties Reference may be had, e.g., to U.S. Pat. No. 4,770,183 (biologically degradable superparamagnetic particles), U.S. Pat. No. 4,810,401 (superparamagnetic solid particles), U.S. Pat. No. 4,824,587 (composites of coercive particles and superparamagnetic particles), U.S. Pat. No. 4,827,945 (biologically degradable superparamagnetic materials), U.S. Pat. No. 4,951,675 (biodegradable superparamagnetic metal oxides), U.S. Pat. No. 4,965,007 (encapsulated superparamagnetic particles), U.S. Pat. No. 5,160,726 (superparamagnetic MR contrast agents), U.S. Pat. No. 5,236,783 (superparamagnetic fine particles), U.S. Pat. No. 5,260,050 (superparamagnetic ferromagnetically coupled chromium complexes), U.S. Pat. No. 5,381,664 (nanocomposite material), U.S. Pat. No. 5,384,109 (diagnostic magnetometry using superparamagnetic particles), U.S. Pat. No. 5,667,924 (superparamagnetic image character recognition compositions), U.S. Pat. No. 6,133,047 (superparamagnetic monodisperse particles), U.S. Pat. No. 6,207,134 (ultrafine lightly coated superparamagnetic particles for MRI), U.S. Pat. No. 6,645,626 (superparamagnetic nanostructured materials), U.S. Pat. No. 6,761,747 (dispersion containing pyrogenically manufactured abrasive particles with superparamagnetic domains), and the like. The entire disclosure of each of these United States patents is hereby incorporated by reference into this specification.

The collection of nanomagnetic particles of this embodiment of the invention is generally comprised of at least about 0.05 weight percent of such nanomagnetic particles and, preferably, at least about 5 weight percent of such nanomagnetic particles. In one embodiment, such collection is comprised of at least about 50 weight percent of such magnetic particles. In another embodiment, such collection consists essentially of such nanomagnetic particles.

The average size of the nanomagnetic particles is preferably less than about 100 nanometers. In one embodiment, the nanomagnetic particles have an average size of less than about 20 nanometers. In another embodiment, the nanomagnetic particles have an average size of less than about 15 nanometers. In yet another embodiment, such average size is less than about 11 nanometers. In yet another embodiment, such average size is less than about 3 nanometers.

In one embodiment of this invention, the nanomagnetic particles have a phase transition temperature of from about 0 degrees Celsius to about 1,200 degrees Celsius. In one aspect of this embodiment, the phase transition temperature is from about 40 degrees Celsius to about 200 degrees Celsius.

As used herein, the term phase transition temperature refers to temperature in which the magnetic order of a magnetic particle transitions from one magnetic order to another. Thus, for example, when a magnetic particle transitions from the ferromagnetic order to the paramagnetic order, the phase transition temperature is the Curie temperature. Thus, e.g., when the magnetic particle transitions from the anti-ferromagnetic order to the paramagnetic order, the phase transition temperature is known as the Neel temperature.

The nanomagnetic material of this invention is well adapted for hyperthermia therapy because, e.g., of the small size of the nanomagnetic particles and the magnetic properties of such particles, such as, e.g., their Curie temperature.

As used herein, the term “Curie temperature” refers to the temperature marking the transition between ferromagnetism and paramagnetism, or between the ferroelectric phase and paraelectric phase. This term is also sometimes referred to as the “Curie point.” Reference may be had, e.g., to U.S. Pat. Nos. 5,429,583, 6,599,234, 6,565,887, 6,267,313, 4,138,998, 5,571,153, 6,635,009, and the like. The entire disclosure of each of these United States patents is hereby incorporated by reference into this specification.

As used herein, the term “Neel temperature” refers to a temperature, characteristic of certain metals, alloys, and salts, below which spontaneous magnetic ordering takes place so that they become antiferromagnetic, and above which they are paramagnetic; this is also known as the Neel point. Reference may be had, e.g., to U.S. Pat. Nos. 4,103,315, 3,791,843, 5,492,720, 6,181,533, 3,883,892, 5,264,980, 3,845,306, 6,083,632, 4,396,886, 6,020,060, and the like. The entire disclosure of each of these United States patents is hereby incorporated by reference into this specification.

In one embodiment, the magnetic order of the nanomagnetic particles of this invention is destroyed at a temperature in excess of the phase transition temperature. This phenomenon is illustrated in FIGS. 4A and 4B.

Referring to FIG. 4A, it will be seen that a multiplicity of nano-sized particles 91 are disposed within a cell 93 which, in the embodiment depicted, is a cancer cell. The particles 91 are subjected to electromagnetic radiation 95 which causes them, in the embodiment depicted, to heat to a temperature sufficient to destroy the cancer cell but insufficient to destroy surrounding cells. The particles 91 are preferably delivered to the cancer cell 93 by one or more of the means described elsewhere in this specification and/or in the prior art.

In the embodiment depicted in FIG. 4A, the temperature of the particles 91 is less than the phase transition temperature of such particles, “T_{transition.}” Thus, in this case, the particles 91 have a magnetic order, i.e., they are either ferromagnetic or superparamagnetic and, thus, are able to receive the external radiation 95 and transform at least a portion of the electromagnetic energy into heat.

When the temperature of the particles 91 exceeds the “Tₜᵣₐₙₛᵢₜᵢₒₙ” temperature (i.e., their phase transition temperature), the magnetic order of such particles is destroyed, and they are no longer able to transform electromagnetic energy into heat. This situation is depicted in FIG. 4B.

When the particles 91 cease transforming electromagnetic energy into heat, they tend to cool and then revert to a temperature below “Tₜᵣₐₙₛᵢₜᵢₒₙ”, as depicted in FIG. 4A. Thus, the particles 91 act as a heat switch, ceasing to transform electromagnetic energy into heat when they exceed their phase transition temperature and resuming such capability when they are cooled below their phase transition temperature. This capability is schematically illustrated in FIG. 3A.

In one embodiment, the phase transition temperature of the nanoparticles is higher than the temperature needed to kill cancer cells but lower than the temperature needed to kill normal cells. In one embodiment of this invention, the phase transition temperature of the nanomagnetic material is less than about 50 degrees Celsius and, preferably, less than about 46 degrees Celsius. In one aspect of this embodiment, such phase transition temperature is less than about 45 degrees Celsius.

The nanomagnetic particles of this invention preferably have a saturation magnetization (“magnetic moment”) of from about 2 to about 3,000 electromagnetic units (emu) per cubic centimeter of material. This parameter may be measured by conventional means. Reference may be had, e.g., to U.S. Pat. No. 5,068,519 (magnetic document validator employing remanence and saturation measurements), U.S. Pat. Nos. 5,581,251, 6,666,930, 6,506,264 (ferromagnetic powder), U.S. Pat. Nos. 4,631,202, 4,610,911, 5,532,095, and the like. The entire disclosure of each of these United States patents is hereby incorporated by reference into this specification.

In one embodiment, the saturation magnetization of the nanomagnetic particles is measured by a SQUID (superconducting quantum interference device). Reference may be had, e.g., to U.S. Pat. No. 5,423,223 (fatigue detection in steel using squid magnetometry), U.S. Pat. No. 6,496,713 (ferromagnetic foreign body detection with background canceling), U.S. Pat. Nos. 6,418,335, 6,208,884 (noninvasive room temperature instrument to measure magnetic susceptibility variations in body tissue), U.S. Pat. No. 5,842,986 (ferromagnetic foreign body screening method), U.S. Pat. Nos. 5,471,139, 5,408,178, and the like. The entire disclosure of each of these United States patents is hereby incorporated by reference into this specification.

In one preferred embodiment, the saturation magnetization of the nanomagnetic particle of this invention is at least 100 electromagnetic units (emu) per cubic centimeter and, more preferably, at least about 200 electromagnetic units (emu) per cubic centimeter. In one aspect of this embodiment, the saturation magnetization of such nanomagnetic particles is at least about 1,000 electromagnetic units per cubic centimeter.

In another embodiment, the nanomagnetic material of this invention is present in the form a film with a saturization magnetization of at least about 2,000 electromagnetic units per cubic centimeter and, more preferably, at least about 2,500 electromagnetic units per cubic centimeter. In this embodiment, the nanomagnetic material in the film preferably has the formula A₁A₂(B)ₓC₁(C₂)_{y}, wherein y is 1, and the C moieties are oxygen and nitrogen, respectively.

In one embodiment of this invention, the composition of one aspect of this invention is comprised of nanomagnetic particles with a specified magnetization. As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

In this embodiment, and in one aspect thereof, the nanomagnetic particles are present within a layer that preferably has a saturation magnetization, at 25 degrees Centigrade, of from about 1 to about 36,000 Gauss, or higher. In one embodiment, the saturation magnetization at room temperature of the nanomagnetic particles is from about 500 to about 10,000 Gauss. For a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification. As will be apparent to those skilled in the art, especially upon studying the aforementioned patents, the saturation magnetization of thin films is often higher than the saturation magnetization of bulk objects.

In one embodiment, it is preferred to utilize a thin film with a thickness of less than about 2 microns and a saturation magnetization in excess of 20,000 Gauss. The thickness of the layer of nanomagnetic material is measured from the bottom surface of the layer that contains such material to the top surface of such layer that contains such material; and such bottom surface and/or such top surface may be contiguous with other layers of material (such as insulating material) that do not contain nanomagnetic particles. In one preferred embodiment, the bottom surface of such layer (and the material within about 1 nanometer of such bottom surface) contains at least 150 percent as much of the A moiety (and preferably at least 200 percent as much of the A moiety) as does the top surface of such layer (and the material within about 1 nanometer of such top surface). An illustration how to obtain such a structure by sputtering with a magnetron is illustrated in FIGS. 38 and 39.

By the appropriate selection of nanomagnetic particles, and the thickness of the films deposited, one may obtain saturation magnetizations of as high as at least about 36,000.

In one preferred embodiment, the thin film/coating made by the process of this invention has a magnetization under magnetic resonance imaging (MRI) conditions of from about 0.1 to about 10 electromagnetic units per cubic centimeter. Such MRI conditions typically involve a direct current field of 2.0 Tesla. When exposed to such direct current magnetic field, the magnetization of one preferred coating of the invention is from about 0.2 to about 1 electromagnetic units per cubic centimeter and, more preferably, from about 0.2 to about 0.8 electromagnetic units per cubic centimeter. In one aspect of this embodiment, the thin film/coating contains from about 2 to about 20 moles of the aforementioned A moiety or moieties (such as, e.g., iron and/or cobalt) by the total number of moles of such A moiety or moieties and the B moiety or moieties (such as aluminum); in another aspect, from about 5-10 mole percent of the A moiety (and more preferably from about 6 to about 8 mole percent of the A moiety) is used by total number of moles of the A moiety and the B moiety.

One may produce the aforementioned thin film by conventional sputtering techniques using a target that is, e.g., comprised of from about 1 to about 20 weight percent of iron by total weight of iron and aluminum, and by using as a gaseous reactant a mixture of nitrogen and oxygen. The product produced via this process will have the formula FeAlNO, wherein the iron is preferably present in a concentration of from about 9 to about 11 weight percent of iron by total weight of iron and aluminum. When the iron is in the form of nanomagnetic particles disposed in a dielectric matrix, it is preferred that more of such iron appears closer to the substrate than away from the substrate.

In one embodiment, the nanomagnetic material has a saturation magnetization of from about 1 to about 36,000 Gauss. In one embodiment, the nanomagnetic material has a saturation magnetization of from about 200 to about 26,000 Gauss.

In one embodiment, the nanomagnetic material also has a coercive force of from about 0.01 to about 5,000 Oersteds. The term coercive force refers to the magnetic field, H, which must be applied to a magnetic material in a symmetrical, cyclically magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these United States patents is hereby incorporated by reference into this specification.

In one embodiment, the nanomagnetic material has a coercive force of from about 0.01 to about 3,000 Oersteds. In yet another embodiment, the nanomagnetic material 103 has a coercive force of from about 0.1 to about 10 Oersteds.

In one embodiment, the nanomagnetic material preferably has a relative magnetic permeability use the term relative magnetic permeability of from about 1 to about 500,000; in one embodiment, such material has a relative magnetic permeability of from about 1.5 to about 260,000. As used in this specification, the term relative magnetic permeability is equal to B/H, and is also equal to the slope of a section of the magnetization curve of the magnetic material. Reference may be had, e.g., to page 4-28 of E. U. Condon et al.'s “Handbook of Physics” (McGraw-Hill Book Company, Inc., New York, 1958).

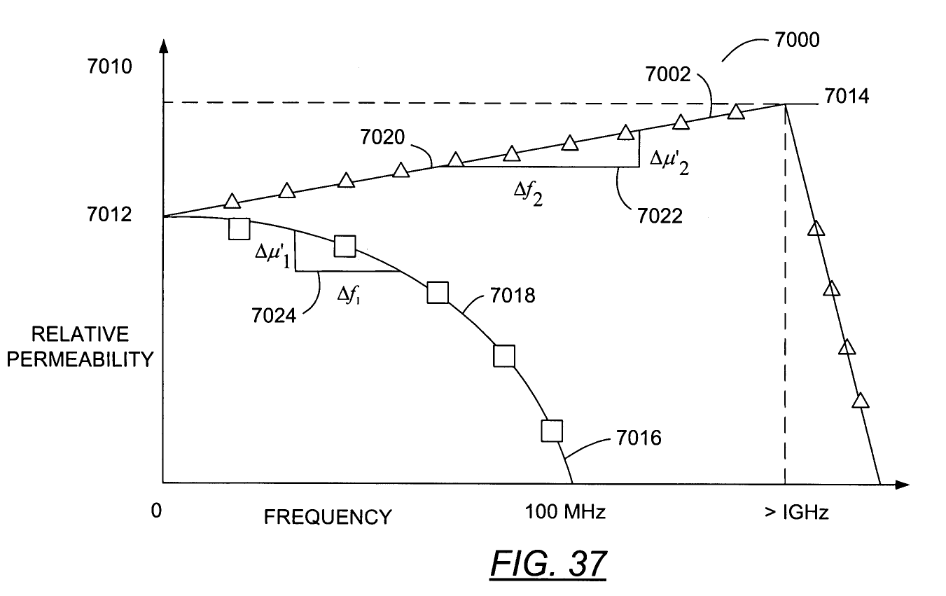

In one embodiment, best illustrated in FIG. 37, when the nanomagnetic material is in the form of a thin film disposed upon a nonmagnetic substrate, the relative magnetic permeability (i.e., the slope of the plot 7020) increases from an alternating current frequency of 10 hertz to a frequency at which the magnetic resonance frequency occurs (at point 7002 in FIG. 37), which generally is at a frequency in excess of 1 gigahertz.

Reference also may be had to page 1399 of Sybil P. Parker's “McGraw-Hill Dictionary of Scientific and Technical Terms,” Fourth Edition (McGraw Hill Book Company, New York, 1989). As is disclosed on this page 1399, permeability is “ . . . a factor, characteristic of a material, that is proportional to the magnetic induction produced in a material divided by the magnetic field strength; it is a tensor when these quantities are not parallel. Reference may also be had to U.S. Pat. No. 6,713,671 (magnetically shielded assembly), U.S. Pat. No. 6,739,999 (magnetically shielded assembly), U.S. Pat. No. 6,844,492 (magnetically shielded conductor), U.S. Pat. No. 6,846,985 (magnetically shielded assembly), the entire disclosure of each of which is hereby incorporated by reference into this specification. Each of these patents utilizes the term “relative magnetic permeability” in its claims.

In one preferred embodiment, the coating of this invention, which preferably is comprised of the aforementioned nanomagnetic material, has a relative alternating current magnetic permeability of at least 1.0 and, more preferably at least about 1.1. (see, e.g., FIG. 37) within the alternating current frequency range of from about 10 megahertz to about 1 gigahertz. In one embodiment, the relative alternating current magnetic permeability of the coating within the aforementioned a.c. frequency range is at least about 1.2 and, more preferably, at least about 1.3. As this term is used in this specification, the relative alternating current magnetic permeability is the relative magnetic permeability of the coating when such coating is subjected to a radio frequency of from about 10 megahertz to about 1 gigahertz. In one aspect of this embodiment, the product of the relative alternating current permeability of the coating (and/or the coated stent) and the relative dielectric constant of the coating (and/or the coated stent) is at least 10 and, more preferably, at least 100. In another aspect of this embodiment, the product of the relative alternating current permeability of the coating (and/or the coated stent) and/or the relative dielectric constant of the coating (and/or the coated stent) is at least about 1,000. In these aspects, the relative dielectric constant may vary, e.g., from about 1 to about 100 and, more preferably from about 7 to about 20. In another aspect, the relative dielectric constant is from about 8 to about 10.

Reference may be had, e.g., to U.S. Pat. Nos. 6,181,232, 5,581,224, 5,506,559, 4,246,586, 6,390,443, and the like. The entire disclosure of each of these United States patents is hereby incorporated by reference into this specification.

In one embodiment, the nanomagnetic material has a relative magnetic permeability of from about 1.5 to about 2,000.

In one embodiment, the nanomagnetic material preferably has a mass density of at least about 0.001 grams per cubic centimeter; in one aspect of this embodiment, such mass density is at least about 1 gram per cubic centimeter. As used in this specification, the term mass density refers to the mass of a give substance per unit volume. See, e.g., page 510 of the aforementioned “McGraw-Hill Dictionary of Scientific and Technical Terms.” In another embodiment, the material has a mass density of at least about 3 grams per cubic centimeter. In another embodiment, the nanomagnetic material has a mass density of at least about 4 grams per cubic centimeter.

In one embodiment, it is preferred that the nanomagnetic material, and/or the article into which the nanomagnetic material has been incorporated, be interposed between a source of radiation and a substrate to be protected therefrom.

In one embodiment, the nanomagnetic material is in the form of a layer that preferably has a saturation magnetization, at 25 degree Centigrade, of from about 1 to about 36,000 Gauss and, more preferably, from about 1 to about 26,000 Gauss. In one aspect of this embodiment, the saturation magnetization at room temperature of the nanomagnetic particles is from about 500 to about 10,000 Gauss.

In one embodiment, the nanomagnetic material is disposed within an insulating matrix so that any heat produced by such particles will be slowly dispersed within such matrix. Such matrix may be made from, e.g., ceria, calcium oxide, silica, alumina, and the like. In general, the insulating material preferably has a thermal conductivity of less than about 20 (calories centimeters/square centimeters-degree Kelvin second)×10,000. See, e.g., page E-6 of the 63^{rd}. Edition of the “Handbook of Chemistry and Physics” (CRC Press, Inc. Boca Raton, Fla., 1982).

Determination of the Heat Shielding Effect of a Magnetic Shield

In one preferred embodiment, the composition of this invention minimizes the extent to which a substrate increases its heat when subjected to a strong magnetic filed. This heat buildup can be determined in accordance with A.S.T.M. Standard Test F-2182-02, “Standard test method for measurement of radio-frequency induced heating near passive implant during magnetic resonance imaging.”

In this test, the radiation used is representative of the fields present during MRI procedures. As is known to those skilled in the art, such fields typically include a static field with a strength of from about 0.5 to about 2 Teslas, a radio frequency alternating magnetic field with a strength of from about 20 microTeslas to about 100 microTeslas, and a gradient magnetic field that has three components (x, y, and z), each of which has a field strength of from about 0.05 to 500 milliTeslas.

During this test, a temperature probe is used to measure the temperature of an unshielded conductor when subjected to the magnetic field in accordance with such A.S.T.M. F-2182-02 test.

The same test is then is then performed upon a shielded conductor assembly that is comprised of the conductor and a magnetic shield.

The magnetic shield used may comprise nanomagnetic particles, as described hereinabove. Alternatively, or additionally, it may comprise other shielding material, such as, e.g., oriented nanotubes (see, e.g., U.S. Pat. No. 6,265,466).

In one embodiment, the shield is in the form of a layer of shielding material with a thickness of from about 10 nanometers to about 1 millimeter. In another embodiment, the thickness is from about 10 nanometers to about 20 microns.

In one preferred embodiment the shielded conductor is an implantable device and is connected to a pacemaker assembly comprised of a power source, a pulse generator, and a controller. The pacemaker assembly and its associated shielded conductor are preferably disposed within a living biological organism.

In one preferred embodiment, when the shielded assembly is tested in accordance with A.S.T.M. 2182-02, it will have a specified temperature increase (“dTₛ”). The “dT_{c}” is the change in temperature of the unshielded conductor using precisely the same test conditions but omitting the shield. The ratio of dTₛ/dT_{c} is the temperature increase ratio; and one minus the temperature increase ratio (1−dTₛ/dT_{c}) is defined as the heat shielding factor.

It is preferred that the shielded conductor assembly have a heat shielding factor of at least about 0.2. In one embodiment, the shielded conductor assembly has a heat shielding factor of at least 0.3.

In one embodiment, the nanomagnetic shield of this invention is comprised of an antithrombogenic material.

Antithrombogenic compositions and structures have been well known to those skilled in the art for many years. Some of these compositions are described, e.g., in applicants' copending patent application U.S. Ser. No. 10/887,521, filed on Jul. 7, 2004, the entire disclosure of which is hereby incorporated by reference into this specification

A Process for Preparation of an Iron-Containing Thin Film

In one preferred embodiment of the invention, a sputtering technique is used to prepare an AlFe thin film or particles, as well as comparable thin films containing other atomic moieties, or particles, such as, e.g., elemental nitrogen, and elemental oxygen. Conventional sputtering techniques may be used to prepare such films by sputtering. See, for example, R. Herrmann and G. Brauer, “D.C.- and R.F. Magnetron Sputtering,” in the “Handbook of Optical Properties: Volume I—Thin Films for Optical Coatings,” edited by R. E. Hummel and K. H. Guenther (CRC Press, Boca Raton, Fla., 1955). Reference also may be had, e.g., to M. Allendorf, “Report of Coatings on Glass Technology Roadmap Workshop,” Jan. 18-19, 2000, Livermore, Calif.; and also to U.S. Pat. No. 6,342,134, “Method for producing piezoelectric films with rotating magnetron sputtering system.” The entire disclosure of each of these prior art documents is hereby incorporated by reference into this specification.

Although the sputtering technique is advantageously used, the plasma technique described elsewhere in this specification also may be used. Alternatively, or additionally, one or more of the other forming techniques described elsewhere in this specification also may be used.

One may utilize conventional sputtering devices in this process. By way of illustration and not limitation, a typical sputtering system is described in U.S. Pat. No. 5,178,739, the entire disclosure of which is hereby incorporated by reference into this specification. As is disclosed in this patent, “ . . . a sputter system 10 includes a vacuum chamber 20, which contains a circular end sputter target 12, a hollow, cylindrical, thin, cathode magnetron target 14, a RF coil 16 and a chuck 18, which holds a semiconductor substrate 19. The atmosphere inside the vacuum chamber 20 is controlled through channel 22 by a pump (not shown). The vacuum chamber 20 is cylindrical and has a series of permanent, magnets 24 positioned around the chamber and in close proximity therewith to create a multiple field configuration near the interior surface 15 of target 12. Magnets 26, 28 are placed above end sputter target 12 to also create a multipole field in proximity to target 12. A singular magnet 26 is placed above the center of target 12 with a plurality of other magnets 28 disposed in a circular formation around magnet 26. For convenience, only two magnets 24 and 28 are shown. The configuration of target 12 with magnets 26, 28 comprises a magnetron sputter source 29 known in the prior art, such as the Torus-10E system manufactured by K. Lesker, Inc. A sputter power supply 30 (DC or RF) is connected by a line 32 to the sputter target 12. A RF supply 34 provides power to RF coil 16 by a line 36 and through a matching network 37. Variable impedance 38 is connected in series with the cold end 17 of coil 16. A second sputter power supply 39 is connected by a line 40 to cylindrical sputter target 14. A bias power supply 42 (DC or RF) is connected by a line 44 to chuck 18 in order to provide electrical bias to substrate 19 placed thereon, in a manner well known in the prior art.”

By way of yet further illustration, other conventional sputtering systems and processes are described in U.S. Pat. No. 5,569,506 (a modified Kurt Lesker sputtering system), U.S. Pat. No. 5,824,761 (a Lesker Torus 10 sputter cathode), U.S. Pat. Nos. 5,768,123, 5,645,910, 6,046,398 (sputter deposition with a Kurt J. Lesker Co. Torus 2 sputter gun), U.S. Pat. Nos. 5,736,488, 5,567,673, 6,454,910, and the like. The entire disclosure of each of these United States patents is hereby incorporated by reference into this specification.

By way of yet further illustration, one may use the techniques described in a paper by Xingwu Wang et al. entitled “Technique Devised for Sputtering AlN Thin Films,” published in “the Glass Researcher,” Volume 11, No. 2 (Dec. 12, 2002).

In one preferred embodiment, a magnetron sputtering technique is utilized, with a Lesker Super System III system The vacuum chamber of this system is preferably cylindrical, with a diameter of approximately one meter and a height of approximately 0.6 meters. The base pressure used is from about 0.001 to 0.0001 Pascals. In one aspect of this process, the target is a metallic FeAl disk, with a diameter of approximately 0.1 meter. The molar ratio between iron and aluminum used in this aspect is approximately 70/30. Thus, the starting composition in this aspect is almost non-magnetic. See, e.g., page 83 (FIG. 3.1aii) of R. S. Tebble et al.'s “Magnetic Materials” (Wiley-Interscience, New York, N.Y., 1969); this Figure discloses that a bulk composition containing iron and aluminum with at least 30 mole percent of aluminum (by total moles of iron and aluminum) is substantially non-magnetic.

In this aspect, to fabricate FeAl films, a DC power source is utilized, with a power level of from about 150 to about 550 watts (Advanced Energy Company of Colorado, model MDX Magnetron Drive). The sputtering gas used in this aspect is argon, with a flow rate of from about 0.0012 to about 0.0018 standard cubic meters per second. To fabricate FeAlN films in this aspect, in addition to the DC source, a pulse-forming device is utilized, with a frequency of from about 50 to about 250 MHz (Advanced Energy Company, model Sparc-le V). One may fabricate FeAlO films in a similar manner but using oxygen rather than nitrogen.

In this aspect, a typical argon flow rate is from about (0.9 to about 1.5)×10⁻³ standard cubic meters per second; a typical nitrogen flow rate is from about (0.9 to about 1.8)×10⁻³ standard cubic meters per second; and a typical oxygen flow rate is from about. (0.5 to about 2)×10⁻³ standard cubic meters per second. During fabrication, the pressure typically is maintained at from about 0.2 to about 0.4 Pascals. Such a pressure range has been found to be suitable for nanomagnetic materials fabrications. In one embodiment, it is preferred that both gaseous nitrogen and gaseous oxygen are present during the sputtering process.

In this aspect, the substrate used may be either flat or curved. A typical flat substrate is a silicon wafer with or without a thermally grown silicon dioxide layer, and its diameter is preferably from about 0.1 to about 0.15 meters. A typical curved substrate is an aluminum rod or a stainless steel wire, with a length of from about 0.10 to about 0.56 meters and a diameter of from (about 0.8 to about 3.0)×10⁻³ meters The distance between the substrate and the target is preferably from about 0.05 to about 0.26 meters.

In this aspect, in order to deposit a film on a wafer, the wafer is fixed on a substrate holder. The substrate may or may not be rotated during deposition. In one embodiment, to deposit a film on a rod or wire, the rod or wire is rotated at a rotational speed of from about 0.01 to about 0.1 revolutions per second, and it is moved slowly back and forth along its symmetrical axis with a maximum speed of about 0.01 meters per second.

In this aspect, to achieve a film deposition rate on the flat wafer of 5×10⁻¹⁰ meters per second, the power required for the FeAl film is 200 watts, and the power required for the FeAlN film is 500 watts The resistivity of the FeAlN film is approximately one order of magnitude larger than that of the metallic FeAl film. Similarly, the resistivity of the FeAlO film is about one order of magnitude larger than that of the metallic FeAl film.

Iron containing magnetic materials, such as FeAl, FeAlN and FeAlO, FeAlNO, FeCoAlNO, and the like, may be fabricated by sputtering. The magnetic properties of those materials vary with stoichiometric ratios, particle sizes, and fabrication conditions; see, e.g., R. S. Tebble and D. J. Craik, “Magnetic Materials”, pp. 81-88, Wiley-Interscience, New York, 1969 As is disclosed in this reference, when the iron molar ratio in bulk FeAl materials is less than 70 percent or so, the materials will no longer exhibit magnetic properties.

However, it has been discovered that, in contrast to bulk materials, a thin film material often exhibits different properties.

In one embodiment, the magnetic material A is dispersed within nonmagnetic material B. This embodiment is depicted schematically in FIG. 5.

Referring to FIG. 5, and in the preferred embodiment depicted therein, it will be seen that A moieties 102, 104, and 106 are preferably separated from each other either at the atomic level and/or at the nanometer level. The A moieties may be, e.g., A atoms, clusters of A atoms, A compounds, A solid solutions, etc. Regardless of the form of the A moiety, it preferably has the magnetic properties described hereinabove.

In the embodiment depicted in FIG. 5, each A moiety preferably produces an independent magnetic moment. The coherence length (L) between adjacent A moieties is, on average, preferably from about 0.1 to about 100 nanometers and, more preferably, from about 1 to about 50 nanometers.

Thus, referring again to FIG. 5, the normalized magnetic interaction between adjacent A moieties 102 and 104, and also between 104 and 106, is preferably described by the formula M=exp(−x/L), wherein M is the normalized magnetic interaction, exp is the base of the natural logarithm (and is approximately equal to 2.71828), x is the distance between adjacent A moieties, and L is the coherence length. M, the normalized magnetic interaction, preferably ranges from about 3×10⁻⁴⁴ to about 1.0. In one preferred embodiment, M is from about 0.01 to 0.99. In another preferred embodiment, M is from about 0.1 to about 0.9.

In one embodiment, and referring again to FIG. 5, x is preferably measured from the center 101 of A moiety 102 to the center 103 of A moiety 104; and x is preferably equal to from about 0.00001 times L to about 100 times L.

In one embodiment, the ratio of x/L is at least 0.5 and, preferably, at least 1.5.

In one embodiment, the “ABC particles” of nanomagnetic material also have a specified coherence length. This embodiment is depicted in FIG. 5A.

As is used with regard to such “ABC particles,” the term “coherence length” refers to the smallest distance 1110 between the surfaces 113 of any particles 115 that are adjacent to each other. It is preferred that such coherence length, with regard to such ABC particles, be less than about 100 nanometers and, preferably, less than about 50 nanometers. In one embodiment, such coherence length is less than about 20 nanometers.

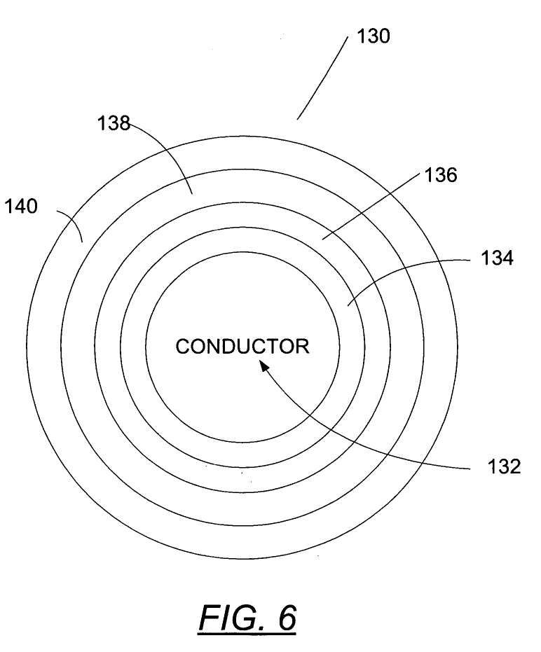

FIG. 6 is a schematic sectional view, not drawn to scale, of a shielded conductor assembly 130 that is comprised of a conductor 132 and, disposed around such conductor, a film 134 of nanomagnetic material. The conductor 132 preferably has a resistivity at 20 degrees Centigrade of from about 1 to about 100-microohom-centimeters.

The film 134 is comprised of nanomagnetic material that preferably has a maximum dimension of from about 10 to about 100 nanometers. The film 134 also preferably has a saturation magnetization of from about 200 to about 26,000 Gauss and a thickness of less than about 2 microns. In one embodiment, the magnetically shielded conductor assembly 130 is flexible, having a bend radius of less than 2 centimeters. Reference may be had, e.g., to U.S. Pat. No. 6,506,972, the entire disclosure of which is hereby incorporated by reference into this specification.

As used in this specification, the term flexible refers to an assembly that can be bent to form a circle with a radius of at least 2 centimeters without breaking. Put another way, the bend radius of the coated assembly is preferably less than 2 centimeters. Reference may be had, e.g., to U.S. Pat. Nos. 4,705,353, 5,946,439, 5,315,365, 4,641,917, 5,913,005, and the like. The entire disclosure of each of these United States patents is hereby incorporated by reference into this specification.

Without wishing to be bound to any particular theory, applicants believe that the use of nanomagnetic materials in their coatings and their articles of manufacture allows one to produce a flexible device that otherwise could not be produced were not the materials so used nano-sized (less than 100 nanometers).

Referring again to FIG. 6, and in the preferred embodiment depicted therein, one or more electrical filter circuit(s) 136 are preferably disposed around the nanomagnetic film 134. These circuit(s) may be deposited by conventional means.

In one embodiment, the electrical filter circuit(s) are deposited onto the film 134 by one or more of the techniques described in U.S. Pat. No. 5,498,289 (apparatus for applying narrow metal electrode), U.S. Pat. No. 5,389,573 (method for making narrow metal electrode), U.S. Pat. No. 5,973,573 (method of making narrow metal electrode), U.S. Pat. No. 5,973,259 (heated tool positioned in the X, Y, and 2-directions for depositing electrode), U.S. Pat. No. 5,741,557 (method for depositing fine lines onto a substrate), and the like. The entire disclosure of each of these United States patents is hereby incorporated by reference into this specification.

Referring again to FIG. 6, and in the preferred embodiment depicted therein, disposed around electrical filter circuit(s) 136 is a second film of nanomagnetic material 138, which may be identical to or different from film layer 134. In one embodiment, film layer 138 provides a different filtering response to electromagnetic waves than does film layer 134.

Disposed around nanomagnetic film layer 138 is a second layer of electrical filter circuit(s) 140. Each of circuit(s) 136 and circuit(s) 140 comprises at least one electrical circuit. It is preferred that the at least two circuits that comprise assembly 130 provide different electrical responses.

As is known to those skilled in the art, at high frequencies the inductive reactance of a coil is great. The inductive reactance (X_{L}) is equal to 2πFL, wherein F is the frequency (in hertz), and L is the inductance (in Henries).

At low-frequencies, by comparison, the capacitative reactance (X_{C}) is high, being equal to 1/2πFC, wherein C is the capacitance in Farads. The impedance of a circuit, Z, is equal to the square root of (R²+[X_{L}−X_{C}]²), wherein R is the resistance, in ohms, of the circuit, and X_{L} and X_{C} are the inductive reactance and the capacitative reactance, respectively, in ohms, of the circuit.

Thus, for any particular alternating frequency electromagnetic wave, one can, by the appropriate selection of values for R, L, and C, pick a circuit that is purely resistive (in which case the inductive reactance is equal to the capacitative reactance at that frequency), is primarily inductive, or is primarily capacitative.

Maximum power transfer occurs at resonance, when the inductance reactance is equal to the capacitative reactance and the difference between them is zero. Conversely, minimum power transfer occurs when the circuit has little resistance in it (all circuits have some finite resistance) but is predominantly inductive or predominantly capacitative.

An LC tank circuit is an example of a circuit in which minimum power is transmitted. A tank circuit is a circuit in which an inductor and capacitor are in parallel; such a circuit appears, e.g., in the output stage of a radio transmitter.

An LC tank circuit exhibits the well-known flywheel effect, in which the energy introduced into the circuit continues to oscillate between the capacitor and inductor after an input signal has been applied; the oscillation stops when the tank-circuit finally loses the energy absorbed, but it resumes when a new source of energy is applied. The lower the inherent resistance of the circuit, the longer the oscillation will continue before dying out.

A typical tank circuit is comprised of a parallel-resonant circuit; and it acts as a selective filter. As is known to those skilled in the art, and as is disclosed in Stan Gibilisco's “Handbook of Radio & Wireless Technology” (McGraw-Hill, New York, N.Y., 1999), a selective filter is a circuit designed to tailor the way an electronic circuit or system responds to signals at various frequencies (see page 62).

The selective filter may be a bandpass filter (see pages 62-63 of the Gibilisco book) that comprises a resonant circuit, or a combination of resonant circuits, designed to discriminate against all frequencies except a specified frequency, or a band of frequencies between two limiting frequencies. In a parallel LC circuit, a bandpass filter shows a high impedance at the desired frequency or frequencies and a low impedance at unwanted frequencies. In a series LC configuration, the filter has a low impedance at the desired frequency or frequencies, and a high impedance at unwanted frequencies.

The selective filter may be a band-rejection filter, also known as a band-stop filter (see pages 63-65 of the Gibilisco book). This band-rejection filter comprises a resonant circuit adapted to pass energy at all frequencies except within a certain range. The attenuation is greatest at the resonant frequency or within two limiting frequencies.

The selective filter may be a notch filter; see page 65 of the Gibilisco book. A notch filter is a narrowband-rejection filter. A properly designed notch filter can produce attenuation in excess of 40 decibels in the center of the notch.

The selective filter may be a high-pass filter; see pages 65-66 of the Gibilisco book. A high-pass filter is a combination of capacitance, inductance, and/or resistance intended to produce large amounts of attenuation below a certain frequency and little or no attenuation above that frequency. The frequency above which the transition occurs is called the cutoff frequency.

The selective filter may be a low-pass filter; see pages 67-68 of the Gibilisco book. A low-pass filter is a combination of capacitance, inductance, and/or resistance intended to produce large amounts of attenuation above a certain frequency and little or no attenuation below that frequency.

In the embodiment depicted in FIG. 6, the electrical circuit is preferably integrally formed with the coated conductor construct. In another embodiment, not shown in FIG. 6, one or more electrical circuits are separately formed from a coated substrate construct and then operatively connected to such construct.

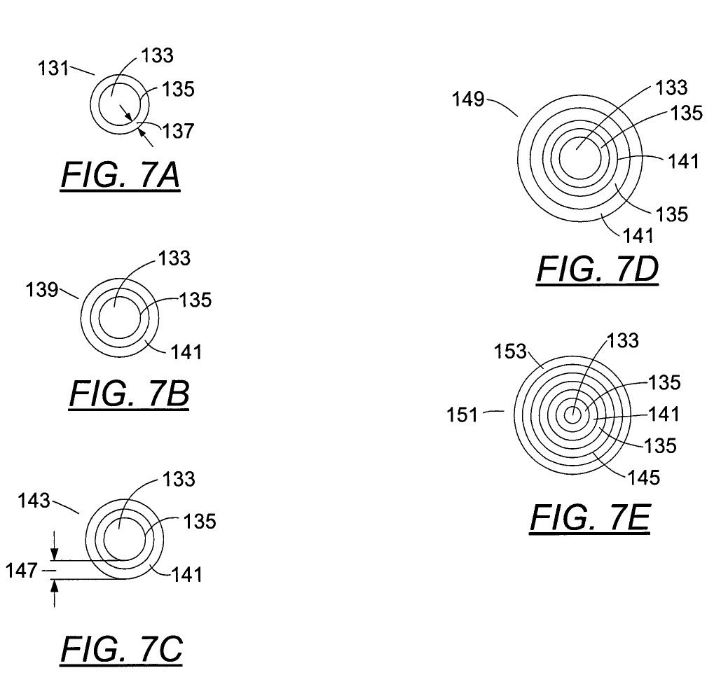

FIG. 7A is a sectional schematic view of one preferred shielded assembly 131 that is comprised of a conductor 133 and, disposed around such conductor 133, a layer of nanomagnetic material 135.

As is used with regard to such “ABC particles,” the term “coherence length” refers to the smallest distance 1110 between the surfaces 113 of any particles 115 that are adjacent to each other. It is preferred that such coherence length, with regard to such ABC particles, be less than about 100 nanometers and, preferably, less than about 50 nanometers. In one embodiment, such coherence length is less than about 20 nanometers. The layer 135 of nanomagnetic material 137 preferably is comprised of nanomagnetic material that may be formed, e.g., by subjecting the material in layer 137 to a magnetic field of from about 10 Gauss to about 40 Tesla for from about 1 to about 20 minutes. The layer 135 preferably has a mass density of at least about 0.001 grams per cubic centimeter (and preferably at least about 0.01 grams per cubic centimeter), a saturation magnetization of from about 1 to about 36,000 Gauss, and a coercive force of from about 0.01 to about 5,000.

In one embodiment, the B moiety is added to the nanomagnetic A moiety, preferably with a B/A molar ratio of from about 5:95 to about 95:5 (see FIG. 3). In one aspect of this embodiment, the resistivity of the mixture of the B moiety and the A moiety is from about 1 micro-ohm-cm to about 10,000 micro-ohm-cm.

Without wishing to be bound to any particular theory, applicants believe that such a mixture of the A and B moieties provides two mechanisms for shielding the magnetic fields. One such mechanism/effect is the shielding provided by the nanomagnetic materials, described elsewhere in this specification. The other mechanism/effect is the shielding provided by the electrically conductive materials.

In one particularly preferred embodiment, the A moiety is iron, the B moiety is aluminum, and the molar ratio of A/B is about 70:30; the resistivity of this mixture is about 8 micro-ohms-cm.

FIG. 7B is a schematic sectional view of a magnetically shielded assembly 139 that is similar to assembly 131 but differs therefrom in that a layer 141 of nanoelectrical material is disposed around layer 135.

The layer of nanoelectrical material 141 preferably has a thickness of from about 0.5 to about 2 microns. In this embodiment, the nanoelectrical material comprising layer 141 has a resistivity of from about 1 to about 100 microohm-centimeters. As is known to those skilled in the art, when nanoelectrical material is exposed to electromagnetic radiation, and in particular to an electric field, it will shield the substrate over which it is disposed from such electrical field. Reference may be had, e.g., to International patent publication WO9820719 in which reference is made to U.S. Pat. No. 4,963,291; each of these patents and patent applications is hereby incorporated by reference into this specification.

As is disclosed in U.S. Pat. No. 4,963,291, one may produce electromagnetic shielding resins comprised of electroconductive particles, such as iron, aluminum, copper, silver and steel in sizes ranging from 0.5 to 50 microns. The entire disclosure of this United States patent is hereby incorporated by reference into this specification.

The nanoelectrical particles used in this aspect of the invention preferably have a particle size within the range of from about 1 to about 100 microns, and a resistivity of from about 1.6 to about 100 microohm-centimeters. In one embodiment, such nanoelectrical particles comprise a mixture of iron and aluminum. In another embodiment, such nanoelectrical particles consist essentially of a mixture of iron and aluminum.

It is preferred that, in such nanoelectrical particles, and in one embodiment, at least 9 moles of aluminum are present for each mole of iron. In another embodiment, at least about 9.5 moles of aluminum are present for each mole of iron. In yet another embodiment, at least 9.9 moles of aluminum are present for each mole of iron.

In one embodiment, and referring again to FIG. 7D, the layer 141 of nanoelectrical material has a thermal conductivity of from about 1 to about 4 watts/centimeter-degree Kelvin.

In one embodiment, not shown, in either or both of layers 135 and 141 there is present both the nanoelectrical material and the nanomagnetic material One may produce such a layer 135 and/or 141 by simultaneously depositing the nanoelectrical particles and the nanomagnetic particles with, e.g., sputtering technology such as, e.g., the sputtering technology described elsewhere in this specification.

FIG. 7C is a sectional schematic view of a magnetically shielded assembly 143 that differs from assembly 131 in that it contains a layer 145 of nanothermal material disposed around the layer 135 of nanomagnetic material. The layer 145 of nanothermal material preferably has a thickness of less than 2 microns and a thermal conductivity of at least about 150 watts/meter-degree Kelvin and, more preferably, at least about 200 watts/meter-degree Kelvin. It is preferred that the resistivity of layer 145 be at least about 1010 microohm-centimeters and, more preferably, at least about 10¹² microohm-centimeters. In one embodiment, the resistivity of layer 145 is at least about 10¹³ microohm centimeters. In one embodiment, the nanothermal layer is comprised of AlN.

In one embodiment, depicted in FIG. 7C, the thickness 147 of all of the layers of material coated onto the conductor 133 is preferably less than about 20 microns.

In FIG. 7D, a sectional view of an assembly 149 is depicted that contains, disposed around conductor 133, layers of nanomagnetic material 135, nanoelectrical material 141, nanomagnetic material 135, and nanoelectrical material 141.

In FIG. 7E, a sectional view of an assembly 151 is depicted that contains, disposed around conductor 133, a layer 135 of nanomagnetic material, a layer 141 of nanoelectrical material, a layer 135 of nanomagnetic material, a layer 145 of nanothermal material, and a layer 135 of nanomagnetic material. Optionally disposed in layer 153 is antithrombogenic material that is biocompatible with the living organism in which the assembly 151 is preferably disposed.

In the embodiments depicted in FIGS. 7A through 7E, the coatings 135, and/or 141, and/or 145, and/or 153, are disposed around a conductor 133. In one embodiment, the conductor so coated is preferably part of medical device, preferably an implanted medical device (such as, e.g., a pacemaker). In another embodiment, in addition to coating the conductor 133, or instead of coating the conductor 133, the actual medical device itself is coated.

A Preferred Sputtering Process

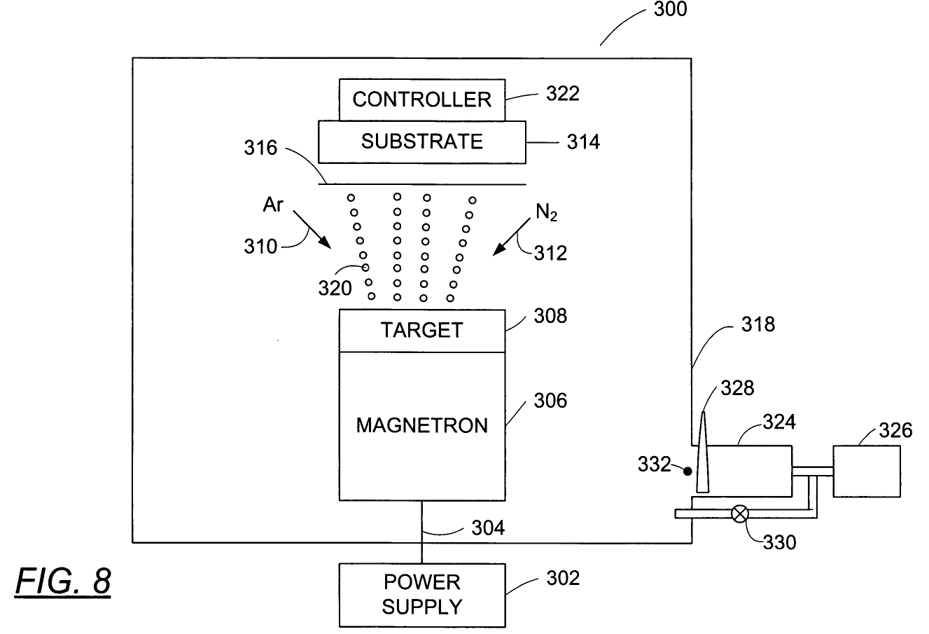

FIG. 8 of the instant specification is substantially identical to FIG. 8 of published United States patent application U.S. 2005/0025797 A1, published on Feb. 3, 2005, the entire disclosure of which is hereby incorporated by reference into this specification. Pages 62-63 of such patent application, without limitation, are specifically incorporated by reference into this specification.

The system depicted in FIG. 8 of the instant specification may be used to prepare an assembly comprised of moieties A, B, and C (see FIG. 4). FIG. 8 will be described hereinafter with reference to one of the preferred ABC moieties, i.e., aluminum nitride doped with magnesium.

FIG. 8 is a schematic of a deposition system 300 comprised of a power supply 302 operatively connected via line 304 to a magnetron 306. Disposed on top of magnetron 306 is a target 308. The target 308 is contacted by gas 310 and gas 312, which cause sputtering of the target 308. The material so sputtered contacts substrate 314 when allowed to do so by the absence of shutter 316.

In one preferred embodiment, the target 308 is mixture of aluminum and magnesium atoms in a molar ratio of from about 0.05 to about 0.5 Mg/(Al+Mg).

The power supply 302 preferably provides pulsed direct current. Generally, power supply 302 provides power in excess of 300 watts, preferably in excess of 500 watts, and more preferably in excess of 1,000 watts. In one embodiment, the power supplied by power supply 302 is from about 1800 to about 2500 watts.

The power supply preferably provides rectangular-shaped pulses with a duration (pulse width) of from about 10 nanoseconds to about 100 nanoseconds. In one embodiment, the pulse width is from about 20 to about 40 nanoseconds.

In between adjacent pulses, preferably substantially no power is delivered. The time between adjacent pulses is generally from about 1 microsecond to about 10 microseconds and is generally at least 100 times greater than the pulse width. In one embodiment, the repetition rate of the rectangular pulses is preferably about 150 kilohertz.

One may use a conventional pulsed direct current (d.c.) power supply. Thus, e.g., one may purchase such a power supply from Advanced Energy Company of Colorado, and/or from ENI Company of Rochester, N.Y.

The pulsed d.c. power from power supply 302 is delivered to a magnetron 306, that creates an electromagnetic field near target 308. In one embodiment, a magnetic field has a magnetic flux density of from about 0.01 Tesla to about 0.1 Tesla. The magnetic flux tends to attract particles (such as particles 320) that also are magnetic.

Because the energy provided to magnetron 306 preferably comprises intermittent pulses, the resulting magnetic fields produced by magnetron 306 will also be intermittent. Without wishing to be bound to any particular theory, applicants believe that the use of such intermittent electromagnetic energy yields better results than those produced by continuous radio-frequency energy.

Referring again to FIG. 8, the process depicted therein preferably is conducted within a vacuum chamber 118 in which the base pressure is from about 1×10⁻⁸ Torr to about 0.000005 Torr. In one embodiment, the base pressure is from about 0.000001 to about 0.000003 Torr.

The temperature in the vacuum chamber 318 generally is ambient temperature prior to the time sputtering occurs.

In one aspect of the embodiment illustrated in FIG. 8, argon gas is fed via line 310, and nitrogen gas is fed via line 312 so that both impact target 308, preferably in an ionized state. In another embodiment of the invention, argon gas, nitrogen gas, and oxygen gas are fed via target 312.

The argon gas, and the nitrogen gas, are fed at flow rates such that the flow rate of the argon gas divided by the flow rate of the nitrogen gas preferably is from about 0.6 to about 1.2. In one aspect of this embodiment, such ratio of argon to nitrogen is from about 0.8 to about 0.95. Thus, for example, the flow rate of the argon may be 20 standard cubic centimeters per minute, and the flow rate of the nitrogen may be 23 standard cubic feet per minute.

The argon gas, and the nitrogen gas, contact a target 308 that is preferably immersed in an electromagnetic field. This field tends to ionize the argon and the nitrogen, providing ionized species of both gases. It is such ionized species that bombard target 308.

In one embodiment, target 308 may be, e.g., pure aluminum. In one preferred embodiment, however, target 308 is aluminum doped with minor amounts of one or more of the aforementioned moieties B.

In the latter embodiment, the moieties B are preferably present in a concentration of from about 1 to about 40 molar percent, by total moles of aluminum and moieties B. It is preferred to use from about 5 to about 30 molar percent of such moieties B.

The ionized argon gas, and the ionized nitrogen gas, after impacting the target 308, creates a multiplicity of sputtered particles 320. In the embodiment illustrated in FIG. 8 the shutter 316 prevents the sputtered particles from contacting substrate 314.

When the shutter 316 is removed, however, the sputtered particles 320 can contact and coat the substrate 314. Depending upon the amount of kinetic energy each of such sputtered particles have, some of such particles are attracted back towards the magnetron 306.

In one embodiment, illustrated in FIG. 8 the temperature of substrate 314 is controlled by controller 322 that can heat the substrate (by means such as a conduction heater or an infrared heater) and/or cool the substrate (by means such as liquid nitrogen or water).

The sputtering operation increases the pressure within the region of the sputtered particles 320. In general, the pressure within the area of the sputtered particles 320 is at least 100 times, and preferably 1000 times, greater than the base pressure.

Referring again to FIG. 8 a cryo pump 324 is preferably used to maintain the base pressure within vacuum chamber 318. In the embodiment depicted, a mechanical pump (dry pump) 326 is operatively connected to the cryo pump 324. Atmosphere from chamber 318 is removed by dry pump 326 at the beginning of the evacuation. At some point, shutter 328 is removed and allows cryo pump 324 to continue the evacuation. A valve 330 controls the flow of atmosphere to dry pump 326 so that it is only open at the beginning of the evacuation.

It is preferred to utilize a substantially constant pumping speed for cryo pump 324, i.e., to maintain a constant outflow of gases through the cryo pump 324. This may be accomplished by sensing the gas outflow via sensor 332 and, as appropriate, varying the extent to which the shutter 328 is open or partially closed.

In one embodiment, the cleaned substrate 314 is presputtered by suppressing sputtering of the target 308 and sputtering the surface of the substrate 314.

As will be apparent to those skilled in the art, the process depicted in FIG. 8 may be used to prepare coated substrates 314 comprised of moieties other than doped aluminum nitride.

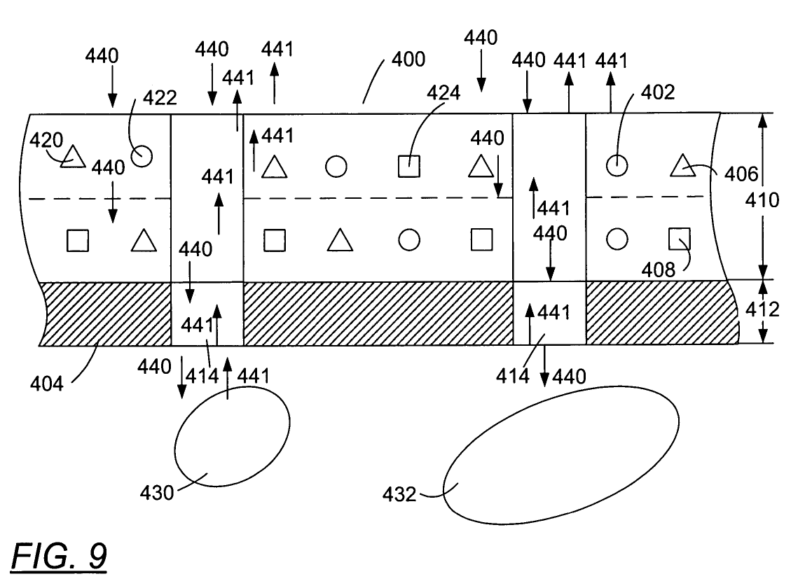

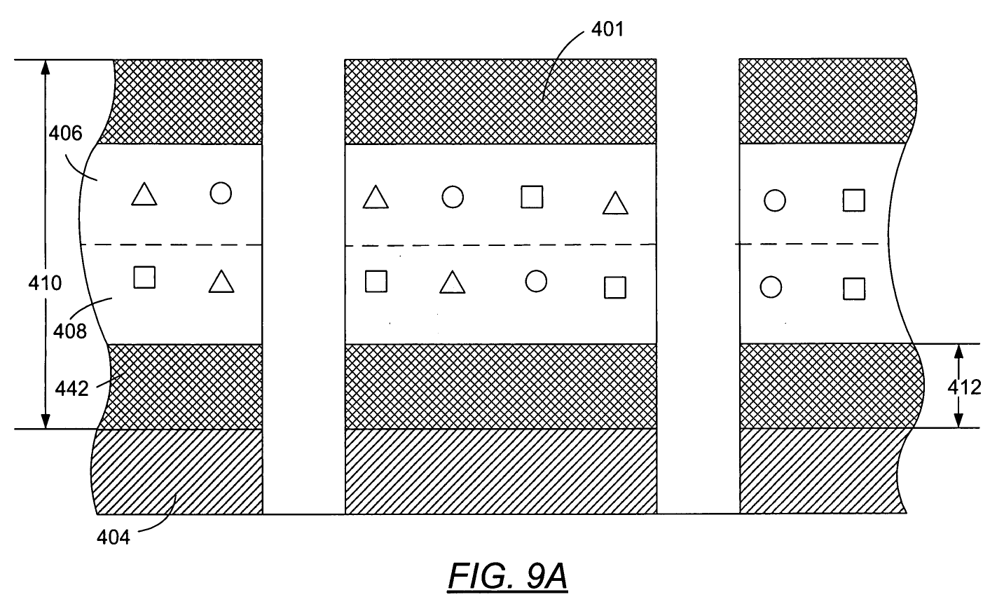

FIG. 9 is a schematic, partial sectional illustration of a coated substrate 400 that, in the preferred embodiment illustrated, is comprised of a coating 402 disposed upon a stent 404. As will be apparent, only one side of the coated stent 404 is depicted for simplicity of illustration. As will also be apparent, the direct current magnetic susceptibility of assembly 400 is equal to the mass of stent (404)×(the susceptibility of stent 404)+the (nmass of the coating 402)×(the susceptibility of coating 402).

In the preferred coated substrate depicted in FIG. 9, the coating 402 may be comprised of one layer of material, two layers of material, or three or more layers of material.

Regardless of the number of coating layers used, it is preferred that the total thickness 410 of the coating 402 be at least about 400 nanometers and, preferably, be from about 400 to about 4,000 nanometers. In one embodiment, thickness 410 is from about 600 to about 1,000 nanometers. In another embodiment, thickness 410 is from about 750 to about 850 nanometers.

In the embodiment depicted, the substrate 404 has a thickness 412 that is substantially greater than the thickness 410. As will be apparent, the coated substrate 400 is not drawn to scale.

In general, the thickness 410 is less than about 5 percent of thickness 412 and, more preferably, less than about 2 percent. In one embodiment, the thickness of 410 is no greater than about 1.5 percent of the thickness 412.

The substrate 404, prior to the time it is coated with coating 402, has a certain flexural strength, and a certain spring constant.

The flexural strength is the strength of a material in bending, i.e., its resistance to fracture. As is disclosed in ASTM C-790, the flexural strength is a property of a solid material that indicates its ability to withstand a flexural or transverse load. As is known to those skilled in the art, the spring constant is the constant of proportionality k which appears in Hooke's law for springs. Hooke's law states that: F=−kx, wherein F is the applied force and x is the displacement from equilibrium. The spring constant has units of force per unit length.

Means for measuring the spring constant of a material are well known to those skilled in the art. Reference may be had, e.g., to U.S. Pat. No. 6,360,589 (device and method for testing vehicle shock absorbers), U.S. Pat. No. 4,970,645 (suspension control method and apparatus for vehicle), U.S. Pat. Nos. 6,575,020, 4,157,060, 3,803,887, 4,429,574, 6,021,579, and the like. The entire disclosure of each of these United States patents is hereby incorporated by reference into this specification.

Referring again to FIG. 9, the flexural strength of the uncoated substrate 404 preferably differs from the flexural strength of the coated substrate 404 by no greater than about 5 percent. Similarly, the spring constant of the uncoated substrate 404 differs from the spring constant of the coated substrate 404 by no greater than about 5 percent.

Referring again to FIG. 9, and in the preferred embodiment depicted, the substrate 404 is comprised of a multiplicity of openings through which biological material is often free to pass. As will be apparent to those skilled in the art, when the substrate 404 is a stent, it will be realized that the stent has a mesh structure.

FIG. 10 is a schematic view of a typical stent 500 that is comprised of wire mesh 502 constructed in such a manner as to define a multiplicity of openings 504. The mesh material is typically a metal or metal alloy, such as, e.g., stainless steel, Nitinol (an alloy of nickel and titanium), niobium, copper, etc.

Typically the materials used in stents tend to cause current flow when exposed to a field 506. When the field 506 is a nuclear magnetic resonance field, it generally has a direct current component, and a radio-frequency component. For MRI (magnetic resonance imaging) purposes, a gradient component is added for spatial resolution.

The material or materials used to make the stent itself has certain magnetic properties such as, e.g., magnetic susceptibility. Thus, e.g., niobium has a magnetic susceptibility of 1.95×10⁻⁶ centimeter-gram-second units. Nitinol has a magnetic susceptibility of from about 2.5 to about 3.8×10⁻⁶ centimeter-gram-second units. Copper has a magnetic susceptibility of from −5.46 to about −6.16×10⁻⁶ centimeter-gram-second units.

The total magnetic susceptibility of an object is equal to the mass of the object times its susceptibility. Thus, assuming an object has equal parts of niobium, Nitinol, and copper, its total susceptibility would be equal to (+1.95+3.15−5.46)×10⁻⁶ cgs, or about 0.36×10⁻⁶ cgs.

In a more general case, where the masses of niobium, Nitinol, and copper are not equal in the object, the susceptibility, in c.g.s. units, would be equal to 1.95 Mn+3.15 Mni−5.46Mc, wherein Mn is the mass of niobium, Mni is the mass of Nitinol, and Mc is the mass of copper.

When any particular material is used to make the stent, its response to an applied MRI field will vary depending upon, e.g., the relative orientation of the stent in relationship to the fields (including the d.c. field, the r.f. field, an the gradient field).

Any particular stent implanted in a human body will tend to have a different orientation than any other stent implanted in another human body due, in part, to the uniqueness of each human body. Thus, it cannot be predicted a priori how any particular stent will respond to a particular MRI field.

The solution provided by one aspect of applicants' invention tends to cancel, or compensate for, the response of any particular stent in any particular body when exposed to an MRI field.

Referring again to FIG. 10, and to the uncoated stent 500 depicted therein, when an MRI field 506 is imposed upon the stent, it will tend to induce eddy currents. As used in this specification, the term eddy currents refers to loop currents and surface eddy currents.

Referring to FIG. 10, the MRI field 506 will induce a loop current 508. As is apparent to those skilled in the art, the MRI field 506 is an alternating current field that, as it alternates, induces an alternating eddy current 508. The radio-frequency field is also an alternating current field, as is the gradient field. By way of illustration, when the d.c. field is about 1.5 Tesla, the r.f. field has frequency of about 64 megahertz. With these conditions, the gradient field is in the kilohertz range, typically having a frequency of from about 2 to about 200 kilohertz.

Applying the well-known right hand rule, the loop current 508 will produce a magnetic field 510 extending into the plane of the paper and designated by an “x. “This magnetic field 510 will tend to oppose the direction of the applied field 506.

Referring again to FIG. 10, when the stent 500 is exposed to the MRI field 506, a surface eddy current will be produced where there is a relatively large surface area of conductive material such as, e.g., at junction 514.

The stent 500 should be constructed to have certain desirable mechanical properties. However, the materials that will provide the desired mechanical properties generally do not have desirable magnetic and/or electromagnetic properties. In an ideal situation, the stent 500 will produce no loop currents 508 and no surface eddy currents 512; in such situation, the stent 500 would have an effective zero magnetic susceptibility. Put another way, ideally the direct current magnetic susceptibility of an ideal stent should be about 0.

A d.c. (“direct current”) magnetic susceptibility of precisely zero is often difficult to obtain. In general, it is sufficient if the d.c. susceptibility of the stent is plus or minus 1×10⁻³ centimeter-gram-seconds (cgs) and, more preferably, plus or minus 1×10⁻⁴ centimeter-gram-seconds. In one embodiment, the d.c. susceptibility of the stent is equal to plus or minus 1×10⁻⁵ centimeter-gram-seconds. In another embodiment, the d.c. susceptibility of the stent is equal to plus or minus 1×10⁻⁶ centimeter-gram-seconds.

In one embodiment, discussed elsewhere in this specification the d.c. susceptibility of the stent in contact with bodily fluid is plus or minus plus or minus 1×10⁻³ centimeter-gram-seconds (cgs), or plus or minus 1×10⁻⁴ centimeter-gram-seconds, or plus or minus 1×10⁻⁵ centimeter-gram-seconds, or plus or minus 1×10⁻⁶ centimeter-gram-seconds. In this embodiment, the materials comprising the nanomagnetic coating on the stent are chosen to have susceptibility values that, in combination with the susceptibility values of the other components of the stent, and of the bodily fluid, will yield the desired values.

The prior art has heretofore been unable to provide such an ideal stent. Applicants' invention allows one to compensate for the deficiencies of the current stents, and/or of the current stents in contact with bodily fluid, by canceling the undesirable effects due to their magnetic susceptibilities, and/or by compensating for such undesirable effects.

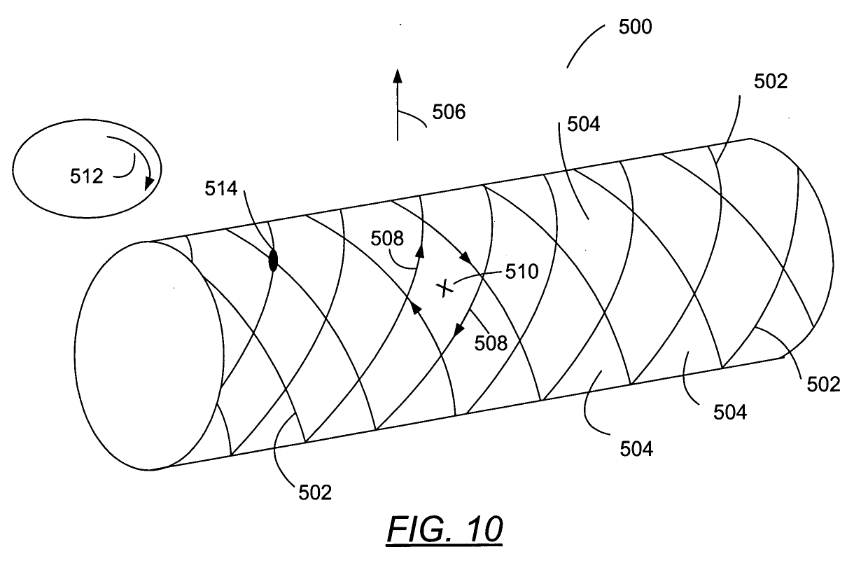

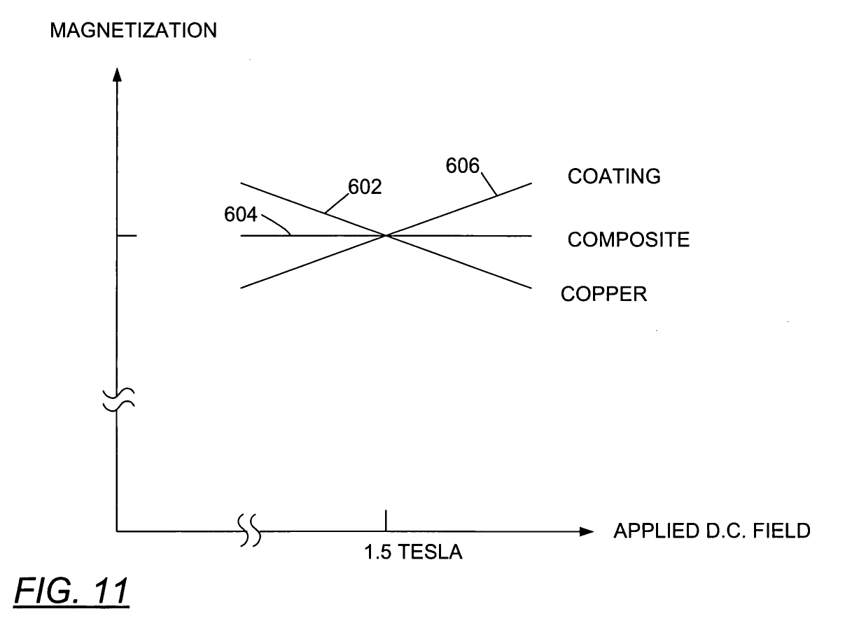

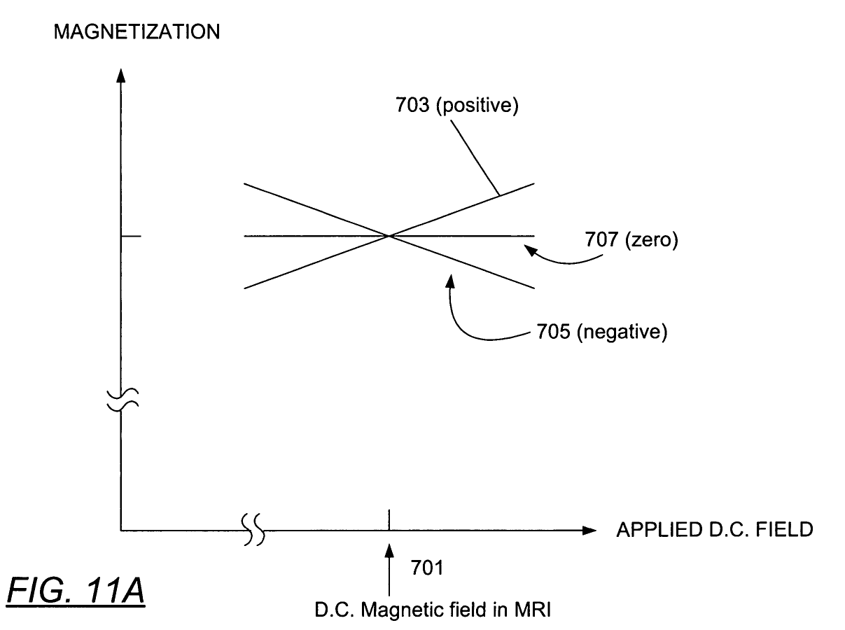

FIG. 11 is a graph of the magnetization of an object (such as an uncoated stent, or a coated stent) when subjected to an electromagnetic filed, such as an MRI field. It will be seen that, at different field strengths, different materials have different magnetic responses.

Thus, e.g., it will be seen that copper, at a d.c. field strength of 1.5 Tesla, is changing its magnetization as a function of the composite field strength (including the d.c. field strength, the r.f. field strength, and the gradient field strength) at a rate (defined by delta-magnetization/delta composite field strength) that is decreasing. With regard to the r.f. field and the gradient field, it should be understood that the order of magnitude of these fields is relatively small compared to the d.c. field, which is usually about 1.5 Tesla.

Referring again to FIG. 11, it will be seen that the slope of line 602 is negative. This negative slope indicates that copper, in response to the applied fields, is opposing the applied fields. Because the applied fields (including r.f. fields, and the gradient fields), are required for effective MRI imaging, the response of the copper to the applied fields tends to block the desired imaging, especially with the loop current and the surface eddy current described hereinabove. The d.c. susceptibility of copper is equal to the mass of the copper present in the device times its magnetic susceptibility.

Referring again to FIG. 11, and in the preferred embodiment depicted therein, the ideal magnetization response is illustrated by line 604, which is the response of the coated substrate of one aspect of this invention, and wherein the slope is substantially zero. As used herein, and with regard to FIG. 11, the term substantially zero includes a slope will produce an effective magnetic susceptibility of from about 1×10⁻⁷ to about 1×10⁻⁸ centimeters-gram-second (cgs).

Referring again to FIG. 11, one means of correcting the negative slope of line 602 is by coating the copper with a coating which produces a response 606 with a positive slope so that the composite material produces the desired effective magnetic susceptibility of from about 1×10⁻⁷ to about 1×10⁻⁸ centimeters-gram-second (cgs) units. In order to do so, the following equation must be satisfied: (magnetic susceptibility of the uncoated device) (mass of uncoated device)+(magnetic susceptibility of copper) (mass of copper)=from about 1×10⁻⁷ to about 1×10⁻⁸ centimeters-gram-second (cgs).

FIG. 9 illustrates a coating that will produce the desired correction for the copper substrate 404. Referring to FIG. 9, it will be seen that, in the embodiment depicted, the coating 402 is comprised of at least nanomagnetic material 420 and nanodielectric material 422.

In one embodiment, the nanomagnetic material 420 preferably has an average particle size of less than about 20 nanometers and a saturation magnetization of from 10,000 to about 26,000 Gauss.

In one embodiment, the nanomagnetic material used is iron. In another embodiment, the nanomagnetic material used is FeAlN. In yet another embodiment, the nanomagnetic material is FeAl. Other suitable materials will be apparent to those skilled in the art and include, e.g., nickel, cobalt, magnetic rare earth materials and alloys, thereof, and the like.

The nanodielectric material 422 preferably has a resistivity at 20 degrees Centigrade of from about 1×10⁻⁵ ohm-centimeters to about 1×10¹³ ohm-centimeters.

Referring again to FIG. 9, and in the preferred embodiment depicted therein, the nanomagnetic material 420 is preferably homogeneously dispersed within nanodielectric material 422, which acts as an insulating matrix. In general, the amount of nanodielectric material 422 in coating 402 exceeds the amount of nanomagnetic material 420 in such coating 402. In general, the coating 402 is comprised of at least about 70 mole percent of such nanodielectric material (by total moles of nanomagnetic material and nanodielectric material). In one embodiment, the coating 402 is comprised of less than about 20 mole percent of the nanomagnetic material, by total moles of nanomagnetic material and nanodielectric material. In one embodiment, the nanodielectric material used is aluminum nitride.

In one preferred embodiment, and referring again to FIG. 9, the nanodielectric material preferably has a dielectric constant of from about 15 to about 10,000 and, more preferably, about 50 to about 5,000. In one embodiment, the dielectric material has a dielectric constant of from about 75 to about 1,500. In another embodiment, the dielectric material has a dielectric constant of from about 100 to about 1,300.

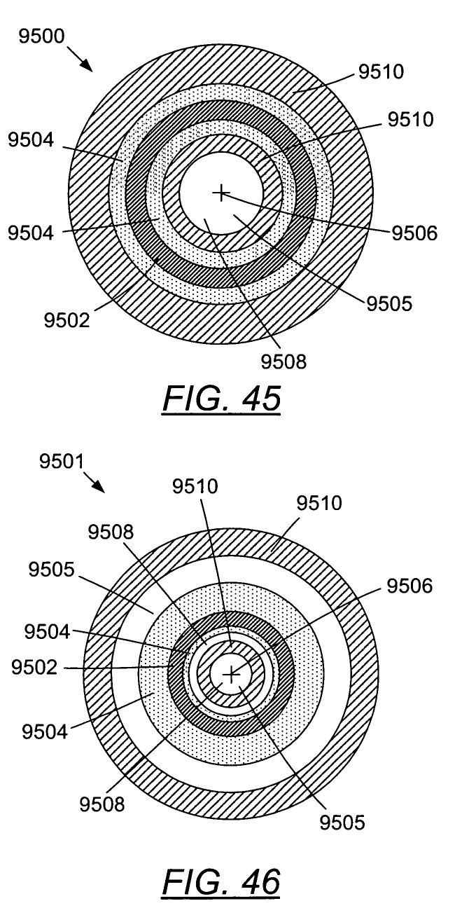

By way of illustration and not limitation, some materials with suitable dielectric constants include, e.g., barium titanate, barium titanate niobate, calcium titanate, cadmium pyroniobate, potassium iodate, potassium niobate, potassium strontium niobate, potassium tanalate niobate, potassium tantalite, lanthanum scandate, lithium niobate, lithium tantalite, manganese niobate, ammonium cadmium sulfate, sodium potassium tartrate tetradeutrate, sodium niobate, lead cobalt tungstate, lead hafnate, lead sulfide, lead selenide, lead telluride, lead titanate, lead zirconate, rubidium nitrate, antimonous selenide, antimonous sulfide iodide, tin antimonide, tin telluride, strontium titanate, titanium dioxide, titanium nitride, and the like. These dielectric materials may be used as a matrix material 422 (see FIG. 9), and/or they may be used as a separate layer of dielectric material (see, e.g., FIG. 45). Regardless of how such dielectric material is used, and in one preferred embodiment, it is preferred to the relative dielectric constant of both the coated stent assembly 400 and the coating disposed on it be from about 1 to about 100.

The term relative dielectric constant is well known to those skilled in the art, and it is defined in (and used in the claims of) each of U.S. Pat. No. 5,307,169 (solid-state imaging device using high relative dielectric constant material as insulating film), U.S. Pat. No. 5,889,696 (thin-film capacitor device and RAM device using ferroelectric film), U.S. Pat. No. 6,352,945 (silicone polymer insulation film on semiconductor substrate), U.S. Pat. No. 6,514,880 (siloxane polymer film on semiconductor substrate), U.S. Pat. No. 6,566,756 (semiconductor device with porous interlayer film), U.S. Pat. No. 6,589,674 (insertion layer for thick film electroluminescent displays), U.S. Pat. No. 6,596,396 (thin-film-like particles having skeleton constructed by carbons and isolated films), U.S. Pat. No. 6,605,515 (method for manufacturing thin-film capacitor), U.S. Pat. No. 6,613,834 (low dielectric constant film material), U.S. Pat. No. 6,645,881 (method of forming a coating film), U.S. Pat. No. 6,737,364 (method for fabricating crystalline-dielectric thin films), U.S. Pat. No. 6,740,974 (semiconductor device having capacitors provided with protective insulating films), U.S. Pat. No. 6,747,334 (thin-film capacitor device), U.S. Pat. No. 6,780,498 (silicon-based composition, low dielectric constant film, and method for producing low dielectric constant film), U.S. Pat. No. 6,812,163 (semiconductor device with porous interlayer insulating film), U.S. Pat. No. 6,828,015 (composite containing thin-film particles having a carbon skeleton), U.S. Pat. No. 6,836,312 (optically transparent film), U.S. Pat. No. 6,852,650 (insulation film on semiconductor substrate), U.S. Pat. No. 6,858,936 (semiconductor device having an improved construction in the interlayer insulating film), and the like. The entire disclosure of each of these United States patents is hereby incorporated by reference into this specification.

Referring again to FIG. 9, and in anther embodiment, not shown, substantially more nanomagnetic material 420 is disposed in the bottom half of such coating than in the top half of such coating; in general, the bottom half of such coating has at least about 1.5 times as much nanomagnetic material 420 as does such top half.

Referring again to FIG. 9, one may optionally include nanoconductive material 424 in the coating 402. This nanoconductive material generally has a resistivity at 20 degrees Centigrade of from about 1×10⁻⁶ ohm-centimeters to about 1×10⁻⁵ ohm-centimeters; and it generally has an average particle size of less than about 100 nanometers. In one embodiment, the nanoconductive material used is aluminum.

Referring again to FIG. 9, and in the embodiment depicted, it will be seen that two layers are preferably used to obtain the desired correction. In one embodiment, three or more such layers are used. This embodiment is depicted in FIG. 9A.

FIG. 9A is a schematic illustration of a coated substrate that is similar to coated substrate 400 but differs therefrom in that it contains two layers of dielectric material 405 and 407. In one embodiment, only one such layer of dielectric material 405 issued. Notwithstanding the use of additional layers 405 and 407, the coating 402 still preferably has a thickness 410 of from about 400 to about 4000 nanometers In the embodiment depicted in FIG. 9A, the direct current susceptibility of the assembly depicted is equal to the sum of the (mass)×(susceptibility) for each individual layer.

As will be apparent, it may be difficult with only one layer of coating material to obtain the desired correction for the material comprising the stent (see FIG. 11). With a multiplicity of layers comprising the coating 402, which may have the same and/or different thicknesses, and/or the same and/or different masses, and/or the same and/or different compositions, and/or the same and/or different magnetic susceptibilities, more flexibility is provided in obtaining the desired correction.

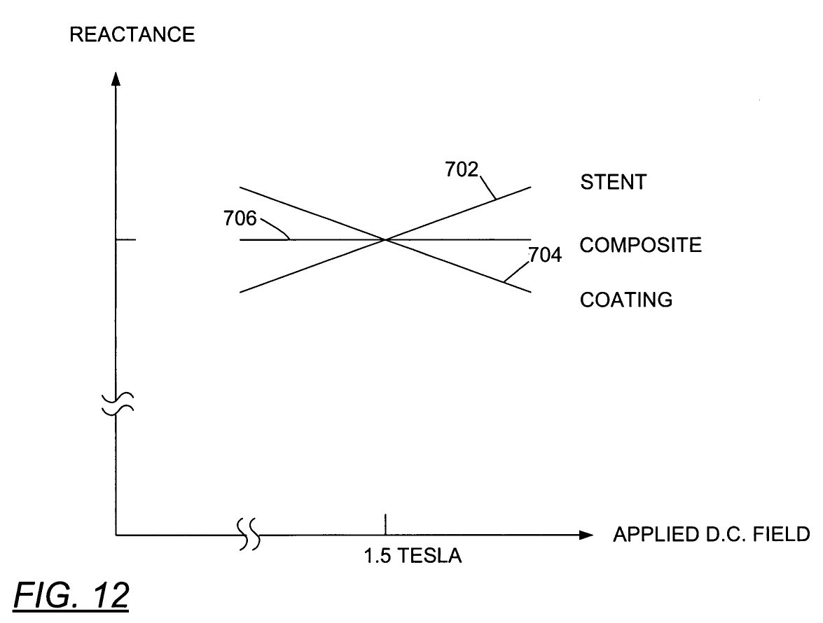

FIG. 11 illustrates the desired correction in terms of magnetization. FIG. 12 illustrates the desired correction in terms of reactance.

Referring again to FIG. 11, in the embodiment depicted a correction is shown for a coating on a substrate. As will be apparent, the same correction can be made with a mixture of at least two different materials in which each of the different materials retains its distinct magnetic characteristics, and/or any composition containing at least two different moieties, provided that each of such different moieties retains its distinct magnetic characteristics. Such correction process is illustrated in FIG. 11A.

FIG. 11A illustrates the response of different species within a composition (such as, e.g., a particle) to magnetic radiation, wherein each such species retains its individual magnetic characteristics. The graph depicted in FIG. 11A does not illustrate the response of different species alloyed with each other, wherein each of the species does not retain its individual magnetic characteristics.

As is known to those skilled in the art, an alloy is a substance having magnetic properties and consisting of two or more elements, which usually are metallic elements. The bonds in the alloy are usually metallic bonds, and thus the individual elements in the alloy do not retain their individual magnetic properties because of the substantial “crosstalk” between the elements via the metallic bonding process.

By comparison, e.g., materials that are covalently bond to each other are more likely to retain their individual magnetic characteristics; it is such materials whose behavior is illustrated in FIG. 11A. Each of the “magnetically distinct” materials may be, e.g., a material in elemental form, a compound, an alloy, etc.

Referring again to FIG. 11A, the response of different, “magnetically distinct” species within a composition (such as particle compact) to MRI radiation is shown. In the embodiment depicted, a direct current (d.c.) magnetic field is shown being applied in the direction of arrow 701. The magnetization plot 703 of the positively magnetized species is shown with a positive slope.

As is known to those skilled in the art, the positively magnetized species include, e.g., those species that exhibit paramagnetism, superparamagnetism, ferromagnetism, and/or ferrimagnetism.

Paramagnetism is a property exhibited by substances which, when placed in a magnetic field, are magnetized parallel to the field to an extent proportional to the field (except at very low temperatures or in extremely large magnetic fields). Paramagnetic materials are well known to those skilled in the art. Reference may be had, e.g., to U.S. Pat. No. 5,578,922 (paramagnetic material in solution), U.S. Pat. No. 4,704,871 (magnetic refrigeration apparatus with belt of paramagnetic material), U.S. Pat. No. 4,243,939 (base paramagnetic material containing ferromagnetic impurity), U.S. Pat. No. 3,917,054 (articles of paramagnetic material), U.S. Pat. No. 3,796,4999 (paramagnetic material disposed in a gas mixture), and the like. The entire disclosure of each of these United States patents is hereby incorporated by reference into this specification.

Superparamagnetic materials are also well known to those skilled in the art. Reference may be had, e.g., to U.S. Pat. No. 5,238,811, the entire disclosure of which is hereby incorporated by reference into this specification, it is disclosed (at column 5) that: “The superparamagnetic material used in the assay methods according to the first and second embodiments of the present invention described above is a substance which has a particle size smaller than that of a ferromagnetic material and retains no residual magnetization after disappearance of the external magnetic field. The superparamagnetic material and ferromagnetic material are quite different from each other in their hysteresis curve, susceptibility, Mesbauer effect, etc. Indeed, ferromagnetic materials are most suited for the conventional assay methods since they require that magnetic micro-particles used for labeling be efficiently guided even when a weak magnetic force is applied. On the other hand, in the non-separation assay method according to the first embodiment of the present invention, it is required that the magnetic-labeled body alone be difficult to guide by a magnetic force, and for this purpose superparamagnetic materials are most suited.” The preparation of these superparamagnetic materials is discussed at columns 6 et seq. of U.S. Pat. No. 5,238,811, wherein it is disclosed that: “The ferromagnetic substances can be selected appropriately, for example, from various compound magnetic substances such as magnetite and gamma-ferrite, metal magnetic substances such as iron, nickel and cobalt, etc. The ferromagnetic substances can be converted into ultramicro particles using conventional methods excepting a mechanical grinding method, i.e., various gas phase methods and liquid phase methods. For example, an evaporation-in-gas method, a laser heating evaporation method, a coprecipitation method, etc. can be applied. The ultramicro particles produced by the gas phase methods and liquid phase methods contain both superparamagnetic particles and ferromagnetic particles in admixture, and it is therefore necessary to separate and collect only those particles which show superparamagnetic property. For the separation and collection, various methods including mechanical, chemical and physical methods can be applied, examples of which include centrifugation, liquid chromatography, magnetic filtering, etc. The particle size of the superparamagnetic ultramicro particles may vary depending upon the kind of the ferromagnetic substance used but it must be below the critical size of single domain particles. Preferably, it is not larger than 10 nm when the ferromagnetic substance used is magnetite or gamma-ferrite and it is not larger than 3 nm when pure iron is used as a ferromagnetic substance, for example.”

Ferromagnetic materials may also be used as the positively magnetized species. As is known to those skilled in the art, ferromagnetism is a property, exhibited by certain metals, alloys, and compounds of the transition (iron group), rare-earth, and actinide elements, in which the internal magnetic moments spontaneously organize in a common direction; this property gives rise to a permeability considerably greater than that of a cuum, and also to magnetic hysteresis. Reference may be had, e.g., to U.S. Pat. Nos. 6,475,650; 6,299,990; 6,690,287 (ferromagnetic material having improved impedance matching); U.S. Pat. No. 6,366,083 (crud layer containing ferromagnetic material on nuclear fuel rods); U.S. Pat. No. 6,011,674 (magnetoresistance effect multilayer film with ferromagnetic film sublayers of different ferromagnetic material compositions); U.S. Pat. No. 5,648,015 (process for preparing ferromagnetic materials); U.S. Pat. Nos. 5,382,304; 5,272,238 (organo-ferromagnetic material); U.S. Pat. No. 5,247,054 (organic polymer ferromagnetic material); U.S. Pat. No. 5,030,371 (acicular ferromagnetic material consisting essentially of iron-containing chromium dioxide); U.S. Pat. No. 4,917,736 (passive ferromagnetic material); U.S. Pat. No. 4,863,715 (contrast agent comprising particles of ferromagnetic material); U.S. Pat. No. 4,835,510 (magnetoresistive element of ferromagnetic material); U.S. Pat. No. 4,739,294 (amorphous and non-amorphous ferromagnetic material); U.S. Pat. No. 4,289,937 (fine grain ferromagnetic material); U.S. Pat. No. 4,023,412 (the Curie point of a ferromagnetic material); U.S. Pat. No. 4,015,030 (stabilized ferromagnetic material); U.S. Pat. No. 4,004,997 (a polymerizable composition containing a magnetized powdered ferromagnetic material); U.S. Pat. No. 3,851,375 (sintered oxidic ferromagnetic material); U.S. Pat. No. 3,850,706 (ferromagnetic materials comprised of transition metals); and the like. The entire disclosure of each of these United States patents is hereby incorporated by reference into this specification.

Ferrimagnetic materials may also be used as the positively magnetized specifies. As is known to those skilled in the art, ferrimagnetism is a type of magnetism in which the magnetic moments of neighboring ions tend to align nonparallel, usually antiparallel, to each other, but the moments are of different magnitudes, so there is an appreciable, resultant magnetization. Reference may be had, e.g., to U.S. Pat. Nos. 6,538,919; 6,056,890 (ferrimagnetic materials with temperature stability); U.S. Pat. Nos. 4,649,495; 4,062,920 (lithium-containing ferrimagnetic materials); U.S. Pat. Nos. 4,059,664; 3,947,372 (ferromagnetic material); U.S. Pat. No. 3,886,077 (garnet structure ferromagnetic material); U.S. Pat. Nos. 3,765,021; 3,670,267; and the like. The entire disclosure of each of these United States patents is hereby incorporated by reference into this specification.

A discussion of certain paramagnetic, superparamagnetic, ferromagnetic, and/or ferromagnetic materials is presented in U.S. Pat. No. 5,238,811, the entire disclosure of which is hereby incorporated by reference into this specification.

By way of yet further illustration, and not limitation, some suitable positively magnetized species include, e.g., iron; iron/aluminum; iron/aluminum oxide; iron/aluminum nitride; iron/tantalum nitride; iron/tantalum oxide; nickel; nickel/cobalt; cobalt/iron; cobalt; samarium; gadolinium; neodymium; mixtures thereof; nano-sized particles of the aforementioned mixtures, where super-paramagnetic properties are exhibited; and the like.

By way of yet further illustration, some of suitable positively magnetized species are listed in the “CRC Handbook of Chemistry and Physics,” 63^{rd} Edition (CRC Press, Inc., Boca-Raton, Fla., 1982-1983). As is discussed on pages E-118 to E-123 of such CRC Handbook, materials with positive susceptibility include, e.g., aluminum, americium, cerium (beta form), cerium (gamma form), cesium, compounds of cobalt, dysprosium, compounds of dysprosium, europium, compounds of europium, gadolium, compounds of gadolinium, hafnium, compounds of holmium, iridium, compounds of iron, lithium, magnesium, manganese, molybdenum, neodymium, niobium, osmium, palladium, plutonium, potassium, praseodymium, rhodium, rubidium, ruthenium, samarium, sodium, strontium, tantalum, technicium, terbium, thorium, thulium, titanium, tungsten, uranium, vanadium, ytterbium, yttrium, and the like.

By way of comparison, and referring again to FIG. 11A, plot 705 of the negatively magnetized species is shown with a negative slope. The negatively magnetized species include those materials with negative susceptibilities that are listed on such pages E-118 to E-123 of the CRC Handbook. By way of illustration and not limitation, such species include, e.g.: antimony; argon; arsenic; barium; beryllium; bismuth; boron; calcium; carbon (dia); chromium; copper; gallium; germanium; gold; indium; krypton; lead; mercury; phosphorous; selenium; silicon; silver; sulfur; tellurium; thallium; tin (gray); xenon; zinc; and the link.

Many diamagnetic materials also are suitable negatively magnetized species. As is known to those skilled in the art, diamagnetism is that property of a material that is repelled by magnets. The term “diamagnetic susceptibility” refers to the susceptibility of a diamagnetic material, which is always negative. Diamagnetic materials are well known to those skilled in the art. Reference may be had, e.g., to U.S. Pat. No. 6,162,364 (diamagnetic objects); U.S. Pat. No. 6,159,271 (diamagnetic liquid); U.S. Pat. No. 5,408,178 (diamagnetic and paramagnetic objects); U.S. Pat. No. 5,315,997 (method of magnetic resonance imaging using diamagnetic contrast); U.S. Pat. Nos. 5,162,301; 5,047,392 (diamagnetic colloids); U.S. Pat. Nos. 5,043,101; 5,026,681 (diamagnetic colloid pumps); U.S. Pat. No. 4,908,347 (diamagnetic flux shield); U.S. Pat. Nos. 4,778,594; 4,735,796; 4,590,922; 4,290,070; 3,899,758; 3,864,824; 3,815,963 (pseudo-diamagnetic suspension); U.S. Pat. Nos. 3,597,022; 3,572,273; and the like. The entire disclosure of each of these United States patents is hereby incorporated by reference into this specification.

By way of further illustration, the diamagnetic material used may be an organic compound with a negative susceptibility. Referring to pages E-123 to pages E-134 of the aforementioned CRC Handbook, such compounds include, e.g.: alanine; allyl alcohol; amylamine; aniline; asparagines; aspartic acid; butyl alcohol; cholesterol; coumarin; diethylamine; erythritol; eucalyptol; fructose; galactose; glucose; D-glucose; glutamic acid; glycerol; glycine; leucine; isoleucine; mannitol; mannose; and the like.

Referring again to FIG. 11A, when a positively magnetized species is mixed with a negatively magnetized species, and assuming that each species retains its magnetic properties, the resulting magnetic properties are indicated by plot 707, with substantially zero magnetization. In this embodiment, one must insure that the positively magnetized species does not lose its magnetic properties, as often happens when one material is alloyed with another. The magnetic properties of alloys and compounds containing different species are known, and thus it readily ascertainable whether the different species that make up such alloys and/or compounds have retained their unique magnetic characteristics.

Without wishing to be bound to any particular theory, applicants believe that, when a positively magnetized species is mixed with a negatively magnetized species, and assuming that each species retains its magnetic properties, the plot 707 (zero magnetization) will be achieved when the volume of the positively magnetized species times its positive susceptibility is substantially equal to the volume of the negatively magnetized species times its negative susceptibility For this relationship to hold, however, each of the positively magnetized species and the negatively magnetized species must retain the distinctive magnetic characteristics when mixed with each other.

Thus, for example, if element A has a positive magnetic susceptibility, and element B has a negative magnetic susceptibility, the alloying of A and B in equal proportions may not yield a zero magnetization compact.

Without wishing to be bound to any particular theory, nano-sized particles, or micro-sized particles (with a size of at least about 0.5 nanometers) tend to retain their magnetic properties as long as they remain in particulate form. On the other hand, alloys of such materials often do not retain such properties.

With regard to reactance (see FIG. 12) the r.f. field and the gradient field are treated as a radiation source which is applied to a living organism comprised of a stent in contact with biological material. The stent, with or without a coating, reacts to the radiation source by exhibiting a certain inductive reactance and a certain capacitative reactance. The net reactance is the difference between the inductive reactance and the capacitative reactance; and it desired that the net reactance be as close to zero as is possible. When the net reactance is greater than zero, it distorts some of the applied MRI fields and thus interferes with their imaging capabilities. Similarly, when the net reactance is less than zero, it also distorts some of the applied MRI fields.

Nullification of the Susceptibility Contribution Due to the Substrate

As will be apparent by reference, e.g., to FIG. 11, the copper substrate depicted therein has a negative susceptibility, the coating depicted therein has a positive susceptibility, and the coated substrate thus has a substantially zero susceptibility. As will also be apparent, some substrates (such niobium, nitinol, stainless steel, etc.) have positive susceptibilities. In such cases, and in one preferred embodiment, the coatings should preferably be chosen to have a negative susceptibility so that, under the conditions of the MRI radiation (or of any other radiation source used), the net susceptibility of the coated object is still substantially zero. As will be apparent, the contribution of each of the materials in the coating(s) is a function of the mass of such material and its magnetic susceptibility.

The magnetic susceptibilities of various substrate materials are well known. Reference may be had, e.g., to pages E-118 to E-123 of the “Handbook of Chemistry and Physics,” 63rd edition (CRC Press, Inc., Boca Raton, Fla., 1974).

Once the susceptibility of the substrate material is determined, one can use the following equation: χ_{sub}+χ_{coat}=0, wherein χ_{sub} is the susceptibility of the substrate, and χ_{coat} is the susceptibility of the coating, when each of these is present in a 1/1 ratio. As will be apparent, the aforementioned equation is used when the coating and substrate are present in a 1/1 ratio. When other ratios are used other than a 1/1 ratio, the volume percent of each component (or its mass) must be taken into consideration in accordance with the equation: (volume percent of substrate×susceptibility of the substrate)+(volume percent of coating×susceptibility of the coating)=0. One may use a comparable formula in which the weight percent of each component is substituted for the volume percent, if the susceptibility is measured in terms of the weight percent.

By way of illustration, and in one embodiment, the uncoated substrate may either comprise or consist essentially of niobium, which has a susceptibility of +195.0×10⁻⁶ centimeter-gram seconds at 298 degrees Kelvin.

In another embodiment, the substrate may contain at least 98 molar percent of niobium and less than 2 molar percent of zirconium. Zirconium has a susceptibility of −122×0×10⁻⁶ centimeter-gram seconds at 293 degrees Kelvin. As will be apparent, because of the predominance of niobium, the net susceptibility of the uncoated substrate will be positive.

The substrate may comprise Nitinol. Nitinol is a paramagnetic alloy, an intermetallic compound of nickel and titanium; the alloy preferably contains from 50 to 60 percent of nickel, and it has a permeability value of about 1.002. The susceptibility of Nitinol is positive.

Nitinols with nickel content ranging from about 53 to 57 percent are known as “memory alloys” because of their ability to “remember” or return to a previous shape upon being heated. which is an alloy of nickel and titanium, in an approximate 1/1 ratio. The susceptibility of Nitinol is positive.

The substrate may comprise tantalum and/or titanium, each of which has a positive susceptibility. See, e.g., the CRC handbook cited above.

When the uncoated substrate has a positive susceptibility, the coating to be used for such a substrate should have a negative susceptibility. Referring again to said CRC handbook, it will be seen that the values of negative susceptibilities for various elements are −9.0 for beryllium, −280.1 for bismuth (s), −10.5 for bismuth (1), −6.7 for boron, −56.4 for bromine (1), −73.5 for bromine(g), −19.8 for cadmium(s), −18.0 for cadmium(1), −5.9 for carbon(dia), −6.0 for carbon (graph), −5.46 for copper(s), −6.16 for copper(1), −76.84 for germanium, −28.0 for gold(s), −34.0 for gold(1), −25.5 for indium, −88.7 for iodine(s), −23.0 for lead(s), −15.5 for lead(1), −19.5 for silver(s), −24.0 for silver(I), −15.5 for sulfur(alpha), −14.9 for sulfur(beta), −15.4 for sulfur(1), −39.5 for tellurium(s), −6.4 for tellurium(1), −37.0 for tin(gray), −31.7 for tin(gray), −4.5 for tin(1), −11.4 for zinc(s), −7.8 for zinc(1), and the like. As will be apparent, each of these values is expressed in units equal to the number in question×10⁻⁶ centimeter-gram seconds at a temperature at or about 293 degrees Kelvin. As will also be apparent, those materials which have a negative susceptibility value are often referred to as being diamagnetic.

By way of further reference, a listing of organic compounds that are diamagnetic is presented on pages E123 to E134 of the aforementioned “Handbook of Chemistry and Physics,” 63rd edition (CRC Press, Inc., Boca Raton, Fla., 1974).

In one embodiment, and referring again to the aforementioned “Handbook of Chemistry and Physics,” 63rd edition (CRC Press, Inc., Boca Raton, Fla., 1974), one or more of the following magnetic materials described below are preferably incorporated into the coating.

The desired magnetic materials, in this embodiment, preferably have a positive susceptibility, with values ranging from +1×10⁻⁶ centimeter-gram seconds at a temperature at or about 293 degrees Kelvin, to about 1×10⁷ centimeter-gram seconds at a temperature at or about 293 degrees Kelvin.

Thus, by way of illustration and not limitation, one may use materials such as Alnicol (see page E-112 of the CRC handbook), which is an alloy containing nickel, aluminum, and other elements such as, e.g., cobalt and/or iron. Thus, e.g., one my use silicon iron (see page E113 of the CRC handbook), which is an acid resistant iron containing a high percentage of silicon. Thus, e.g., one may use steel (see page 117 of the CRC handbook). Thus, e.g., one may use elements such as dyprosium, erbium, europium, gadolinium, hafnium, holmium, manganese, molybdenum, neodymium, nickel-cobalt, alloys of the above, and compounds of the above such as, e.g., their oxides, nitrides, carbonates, and the like.

Referring to FIG. 12, and to the embodiment depicted therein, it will be seen that the uncoated stent has an effective inductive reactance at a d.c. field of 1.5 Tesla that exceeds its capacitative reactance, whereas the coating 704 has a capacitative reactance that exceeds its inductive reactance. The coated (composite) stent 706 has a net reactance that is substantially zero.

As will be apparent, the effective inductive reactance of the uncoated stent 702 may be due to a multiplicity of factors including, e.g., the positive magnetic susceptibility of the materials which it is comprised of it, the loop currents produced, the surface eddy produced, etc. Regardless of the source(s) of its effective inductive reactance, it can be “corrected” by the use of one or more coatings which provide, in combination, an effective capacitative reactance that is equal to the effective inductive reactance.

Referring again to FIG. 9, and in the embodiment depicted, plaque particles 430, 432 are disposed on the inside of substrate 404. When the net reactance of the coated substrate 404 is essentially zero, the imaging field 440 can pass substantially unimpeded through the coating 402 and the substrate 404 and interact with the plaque particles 430/432 to produce imaging signals 441.

The imaging signals 441 are able to pass back through the substrate 404 and the coating 402 because the net reactance is substantially zero. Thus, these imaging signals are able to be received and processed by the MRI apparatus.

Thus, by the use of applicants' technology, one may negate the negative substrate effect and, additionally, provide pathways for the image signals to interact with the desired object to be imaged (such as, e.g., the plaque particles) and to produce imaging signals that are capable of escaping the substrate assembly and being received by the MRI apparatus.

The Product of the Relative Permeability and the Relative Dielectric Constant

Referring again to FIG. 9, the coating 402/404 assembly has a certain relative permeability, and it also has a certain relative dielectric constant. Each of these parameters is discussed elsewhere in this specification.

In one preferred embodiment, the product of the (relative permeability of the coating 402/404 assembly)×(relative dielectric constant of the coating 402/404) assembly is at least 10 and, more preferably, at least 100; thus, e.g., wherein the relative permeability of the 402/404 assembly is 1, the relative dielectric constant of the 404/404 assembly should be at least 10.

In one embodiment, the product of the (relative permeability of the coating 402/404 assembly)×(relative dielectric constant of the coating 402/404) assembly is at least 1,000 and, more preferably, 5,000. In one aspect of this embodiment, the product of the (relative permeability of the coating 402/404 assembly)×(relative dielectric constant of the coating 402/404) assembly is at least 10,000.

In one preferred embodiment, one may ignore the contributions of the substrate to the relative dielectric constant. Thus in this embodiment, the product of the (relative permeability of the coating 402/404 assembly)×(relative dielectric constant of the coating 402) assembly is at least 10 and, more preferably, at least 100; thus, e.g., wherein the relative permeability of the 402/404 assembly is 1, the relative dielectric constant of the 402/404 assembly should be at least 10.

In one embodiment, the product of the (relative permeability of the coating 402/404 assembly)×(relative dielectric constant of the coating 402) assembly is at least 1,000 and, more preferably, 5,000. In one aspect of this embodiment, the product of the (relative permeability of the coating 402/404 assembly)×(relative dielectric constant of the coating 402) assembly is at least 10,000.

In one preferred embodiment, one may ignore the contributions of the substrate to the relative permeability. Thus in this embodiment, the product of the (relative permeability of the coating 402)×(relative dielectric constant of the coating 402/404 assembly) is at least 10 and, more preferably, at least 100; thus, e.g., wherein the relative permeability of the 402 coating is 1, the relative dielectric constant of the 402/404 assembly should be at least 10.

In one embodiment, the product of the (relative permeability of the coating 402 assembly)×(relative dielectric constant of the coating 402/404 assembly) is at least 1,000 and, more preferably, 5,000. In one aspect of this embodiment, the product of the (relative permeability of the coating 402)×(relative dielectric constant of the coating 402/404 assembly) is at least 10,000.

In one preferred embodiment, one may ignore the contributions of the substrate to both the relative permeability and the relative dielectric constant. Thus in this embodiment, the product of the (relative permeability of the coating 402)×(relative dielectric constant of the coating 402) assembly is at least 10 and, more preferably, at least 100; thus, e.g., wherein the relative permeability of the 402 coating is 1, the relative dielectric constant of the coating 402 should be at least 10.

In one embodiment, the product of the (relative permeability of the coating 402 assembly)×(relative dielectric constant of the coating 402) assembly is at least 1,000 and, more preferably, 5,000. In one aspect of this embodiment, the product of the (relative permeability of the coating 402)×(relative dielectric constant of the coating 402) is at least 10,000.

Incorporation of Disclosure of U.S. Ser. No. 10/303/264, filed on Nov. 25, 2002

Pages 69-73 of published United States patent application U.S. 2005/0025797 A1, published on Feb. 3, 2005, presents certain disclosure that was also present in U.S. Pat. No. 6,713,671. The entire disclosure of such published patent application (including, without limitation, pages 69-73 thereof), and the entire disclosure of U.S. Pat. No. 6,713,671, each is incorporated by reference into this specification.

U.S. Pat. No. 6,713,671 discloses a shielded assembly comprised of a substrate and, disposed above a substrate, a shield comprising from about 1 to about 99 weight percent of a first nanomagnetic material, and from about 99 to about 1 weight percent of a second material with a resistivity of from about 1 microohm-centimeter to about 1×1025 microohm centimeters; the nanomagnetic material comprises nanomagnetic particles, and these nanomagnetic particles respond to an externally applied magnetic field by realigning to the externally applied field. Such a shielded assembly and/or the substrate thereof and/or the shield thereof may be used in the processes, compositions, and/or constructs of this invention.

The substrate used may be, e.g, comprised of one or more conductive material(s) that have a resistivity at 20 degrees Centigrade of from about 1 to about 100 microohm-centimeters. Thus, e.g., the conductive material(s) may be silver, copper, aluminum, alloys thereof, mixtures thereof, and the like.

In order to function optimally, the nanomagnetic material should preferably have a specified magnetization. As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

Referring again to FIG. 4 of U.S. Pat. No. 6,713,671, the entire disclosure of which is hereby incorporated by reference into this specification, the layer of nanomagnetic particles 24 preferably has a saturation magnetization, at 25 degrees Centigrade, of from about 1 to about 36,000 Gauss, or higher. In one embodiment, the saturation magnetization at room temperature of the nanomagnetic particles is from about 500 to about 10,000 Gauss. For a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification.

In one embodiment, it is preferred to utilize a thin film with a thickness of less than about 2 microns and a saturation magnetization in excess of 20,000 Gauss.

Referring again to FIG. 4 of U.S. Pat. No. 6,713,671, the nanomagnetic particles 24 are disposed within an insulating matrix so that any heat produced by such particles will be slowly dispersed within such matrix. Such matrix, as indicated hereinabove, may be made from ceria, calcium oxide, silica, alumina. In general, the insulating material 42 preferably has a thermal conductivity of less than about 20 (caloriescentimeters/square centimeters—degree second)×10,000. See, e.g., page E-6 of the 63rd Edition of the “Handbook of Chemistry and Physics” (CRC Press, Inc., Boca Raton, Fla., 1982).

In one preferred embodiment of this invention, and referring to FIG. 6 of U.S. Pat. No. 6,713,671, a film of nanomagnetic material is disposed above at least one surface of a conductor. Referring to such FIG. 6, and in the schematic diagram depicted therein, a source of electromagnetic radiation 100 emits radiation 102 in the direction of film 104. Film 104 is disposed above conductor 106, i.e., it is disposed between conductor 106 of the electromagnetic radiation 102.

Referring again to FIG. 6 of U.S. Pat. No. 6,713,671, the film 104 is adapted to reduce the magnetic field strength at point 108 (which is disposed less than 1 centimeter above film 104) by at least about 50 percent. Thus, if one were to measure the magnetic field strength at point 108, and thereafter measure the magnetic field strength at point 110 (which is disposed less than 1 centimeter below film 104), the latter magnetic field strength would be no more than about 50 percent of the former magnetic field strength. Put another way, the film 104 has a magnetic shielding factor of at least about 0.5.

Referring again to FIG. 6 of U.S. Pat. No. 6,713,671, in one embodiment, the film 104 has a magnetic shielding factor of at least about 0.9, i.e., the magnetic field strength at point 110 is no greater than about 10 percent of the magnetic field strength at point 108. Thus, e.g., the static magnetic field strength at point 108 can be, e.g., one Tesla, whereas the static magnetic field strength at point 110 can be, e.g., 0.1 Tesla. Furthermore, the time-varying magnetic field strength of a 100 milliTesla would be reduced to about 10 milliTesla of the time-varying field.

Referring again to FIG. 6 of U.S. Pat. No. 6,713,671, in one embodiment of the invention of this patent application it is desired to allow as much as the MRI radiation through the stent as is possible so that it can interact with material within the stent. In this embodiment, and by the appropriate choice of the A,B, and C moieties, the preferred film 104 has a magnetic shielding factor of less than about 0.1, i.e., the magnetic field strength at point 110 is at least 90 percent of the magnetic field strength at point 108

Referring again to FIG. 6 of U.S. Pat. No. 6,713,671, the nanomagnetic material 103 in film 104 also has a coercive force of from about 0.01 to about 5,000 Oersteds. The term coercive force refers to the magnetic field, H, which must be applied to a magnetic material in a symmetrical, cyclically magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these United States patents is hereby incorporated by reference into this specification.

Referring again to FIG. 6 of U.S. Pat. No. 6,713,671, in one embodiment, the nanomagnetic material 103 has a coercive force of from about 0.01 to about 3,000 Oersteds. In yet another embodiment, the nanomagnetic material 103 has a coercive force of from about 0.1 to about 10.

Referring again to such FIG. 6, the nanomagnetic material 103 in film 104 preferably has a relative magnetic permeability of from about 1 to about 500,000; in one embodiment, such material 103 has a relative magnetic permeability of from about 1.5 to about 260,000. As used in this specification, the term relative magnetic permeability is equal to B/H, and is also equal to the slope of a section of the magnetization curve of the film. Reference may be had, e.g., to page 4-28 of E. U. Condon et al.'s “Handbook of Physics” (McGraw-Hill Book Company, Inc., New York, 1958). The relative alternating current magnetic permeability is the permeability of the film when it is subjected to an alternating current of 64 megahertz.

Reference also may be had to page 1399 of Sybil P. Parker's “McGraw-Hill Dictionary of Scientific and Technical Terms,” Fourth Edition (McGraw Hill Book Company, New York, 1989). As is disclosed on this page 1399, permeability is “ . . . a factor, characteristic of a material, that is proportional to the magnetic induction produced in a material divided by the magnetic field strength; it is a tensor when these quantities are not parallel.”

Reference also may be had, e.g., to U.S. Pat. Nos. 6,181,232, 5,581,224, 5,506,559, 4,246,586, 6,390,443, and the like. The entire disclosure of each of these United States patents is hereby incorporated by reference into this specification.

In one embodiment, the nanomagnetic material 103 in film 104 has a relative magnetic permeability of from about 1.5 to about 2,000.

Referring to FIG. 8A of U.S. Pat. No. 6,713,671, the nanomagnetic material 202 may be disposed within an insulating matrix (not shown) so that any heat produced by such particles will be slowly dispersed within such matrix. Such matrix, as indicated hereinabove, may be made from ceria, calcium oxide, silica, alumina, and the like. In general, the insulating material 202 preferably has a thermal conductivity of less than about 20 (calories centimeters/square centimeters-degree second)×10,000. See, e.g., page E-6 of the 63rd. Edition of the “Handbook of Chemistry and Physics” (CRC Press, Inc. Boca Raton, Fla., 1982).

In one embodiment, the insulating matrix 202 has the dielectric properties described elsewhere in this specification.

FIG. 11 of U.S. Pat. No. 6,713,671 is a schematic sectional view of a substrate 401, which is part of an implantable medical device (not shown). Referring to such FIG. 11, and in the preferred embodiment depicted therein, it will be seen that substrate 401 is coated with a layer 404 of nanomagnetic material(s). The layer 404, in the embodiment depicted, is comprised of nanomagnetic particulate 405 and nanomagnetic particulate 406. Each of the nanomagnetic particulate 405 and nanomagnetic particulate 406 preferably has an elongated shape, with a length that is greater than its diameter. In one aspect of this embodiment, nanomagnetic particles 405 have a different size than nanomagnetic particles 406. In another aspect of this embodiment, nanomagnetic particles 405 have different magnetic properties than nanomagnetic particles 406. Referring again to such FIG. 11, and in the preferred embodiment depicted therein, nanomagnetic particulate material 405 and nanomagnetic particulate material 406 are designed to respond to an static or time-varying electromagnetic fields or effects in a manner similar to that of liquid crystal display (LCD) materials. More specifically, these nanomagnetic particulate materials 405 and nanomagnetic particulate materials 406 are designed to shift alignment and to effect switching from a magnetic shielding orientation to a non-magnetic shielding orientation. As will be apparent, the magnetic shield provided by layer 404, can be turned “ON” and “OFF” upon demand. In yet another embodiment (not shown), the magnetic shield is turned on when heating of the shielded object is detected.

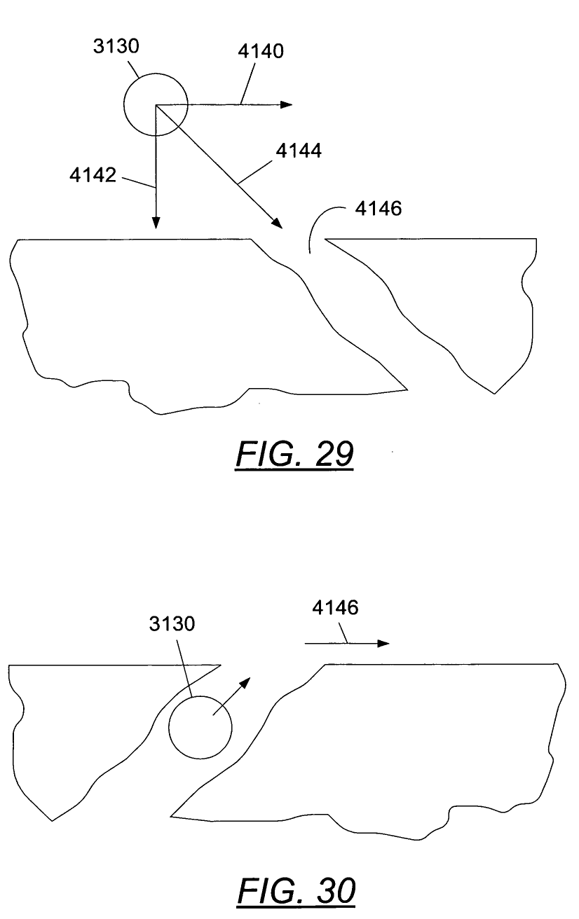

FIG. 29 of U.S. Pat. No. 6,713,671 is a schematic of a preferred shielded assembly 3000 that is comprised of a substrate 3002. The substrate 3002 may be e.g., a foil comprised of metallic material and/or polymeric material. The substrate 3002 may, e.g., comprise ceramic material, glass material, composites, etc. The substrate 3002 may be in the shape of a cylinder, a sphere, a wire, a rectilinear shaped device (such as a box), an irregularly shaped device, etc.

Referring again to FIG. 29 of U.S. Pat. No. 6,713,67, and in one embodiment, the substrate 3002 preferably a thickness of from about 100 nanometers to about 2 centimeters. In one aspect of this embodiment, the substrate 3002 preferably is flexible.

Referring again to FIG. 29 of U.S. Pat. No. 6,713,671, and in the preferred embodiment depicted therein, it will be seen that a shield 3004 is disposed above the substrate 3002. As used herein, the term “above” refers to a shield that is disposed between a source 3006 of electromagnetic radiation and the substrate 3002.

The shield 3004 is comprised of from about 1 to about 99 weight percent of nanomagnetic material 3008; such nanomagnetic material, and its properties, are described elsewhere in this specification. In one embodiment, the shield 3004 is comprised of at least about 40 weight percent of such nanomagnetic material 3008. In another embodiment, the shield 3004 is comprised of at least about 50 weight percent of such nanomagnetic material 3008.

Referring again to FIG. 29 of such U.S. Pat. No. 6,713,671, and in the preferred embodiment depicted therein, it will be seen that the shield 3004 is also comprised of another material 3010 that preferably has an electrical resistivity of from about 1 microohm-centimeter to about 1×1025 microohm-centimeters. This material 3010 is preferably present in the shield at a concentration of from about 1 to about 1 to about 99 weight percent and, more preferably, from about 40 to about 60 weight percent.

In one embodiment, the material 3010 has a dielectric constant of from about 1 to about 50 and, more preferably, from about 1.1 to about 10. In another embodiment, the material 3010 has resistivity of from about 3 to about 20 microohm-centimeters.

In one embodiment, the material 3010 preferably is a nanoelectrical material with a particle size of from about 5 nanometers to about 100 nanometers.

In another embodiment, the material 3010 has an elongated shape with an aspect ratio (its length divided by its width) of at least about 10. In one aspect of this embodiment, the material 3010 is comprised of a multiplicity of aligned filaments.

In one embodiment, the material 3010 is comprised of one or more of the compositions of U.S. Pat. No. 5,827,997 and 5,643,670.

In one embodiment, the medical devices described elsewhere in this specification are coated with a coating that provides specified “signature” when subjected to the MRI field, regardless of the orientation of the device. Such a medical device may be the sealed container 12 (see FIG. 1), a stent, etc. For the purposes of simplicity of description, the coating of a stent will be described, it being understood that the same technology could be used to coat other medical devices. Th effect of such coating is illustrated in FIG. 13.

FIG. 13 is a plot of the image response of the MRI apparatus (image clarity) as a function of the applied MRI fields. The image clarity is generally related to the net reactance.

Referring to FIG. 13, plot 802 illustrates the response of a particular uncoated stent in a first orientation in a patient's body. As will be seen from plot 802, this stent in this first orientation has an effective net inductive response.

FIG. 13, and in particular plot 804, illustrates the response of the same uncoated stent in a second orientation in a patient's body. As has been discussed elsewhere in this specification, the response of an uncoated stent is orientation specific. Thus, plot 804 shows a smaller inductive response than plot 802.

When the uncoated stent is coated with the appropriate coating, as described elsewhere in this specification, the net reactive effect is zero, as is illustrated in plot 806. In this plot 806, the magnetic response of the substrate is nullified regardless of the orientation of such substrate within a patient's body.

In one embodiment, illustrated as plot 808, a stent is coated in such a manner that its net reactance is substantially larger than zero, to provide a unique imaging signature for such stent. Because the imaging response of such coated stent is also orientation independent, one may determine its precise location in a human body with the use of conventional MRI imaging techniques. In effect, the coating on the stent 808 acts like a tracer, enabling one to locate the position of the stent 808 at will.

In one embodiment, if one knows the MRI signature of a stent in a certain condition, one may be able to determine changes in such stent. Thus, for example, if one knows the signature of such stent with plaque deposited on it, and the signature of such stent without plaque deposited on it, one may be able to determine a human body's response to such stent.

Preparation of Coatings Comprised of Nanoelectrical Material

In this portion of the specification, coatings comprised of nanoelectrical material will be described. In accordance with one aspect of this invention, there is provided a nanoelectrical material with an average particle size of less than 100 nanometers, a surface area to volume ratio of from about 0.1 to about 0.05 1/nanometer, and a relative dielectric constant of less than about 1.5.

The nanoelectrical particles of aspect of the invention have an average particle size of less than about 100 nanometers. In one embodiment, such particles have an average particle size of less than about 50 nanometers. In yet another embodiment, such particles have an average particle size of less than about 10 nanometers.

The nanoelectrical particles of this invention have surface area to volume ratio of from about 0.1 to about 0.05 1/nanometer.

When the nanoelectrical particles of this invention are agglomerated into a cluster, or when they are deposited onto a substrate, the collection of particles preferably has a relative dielectric constant of less than about 1.5. In one embodiment, such relative dielectric constant is less than about 1.2.

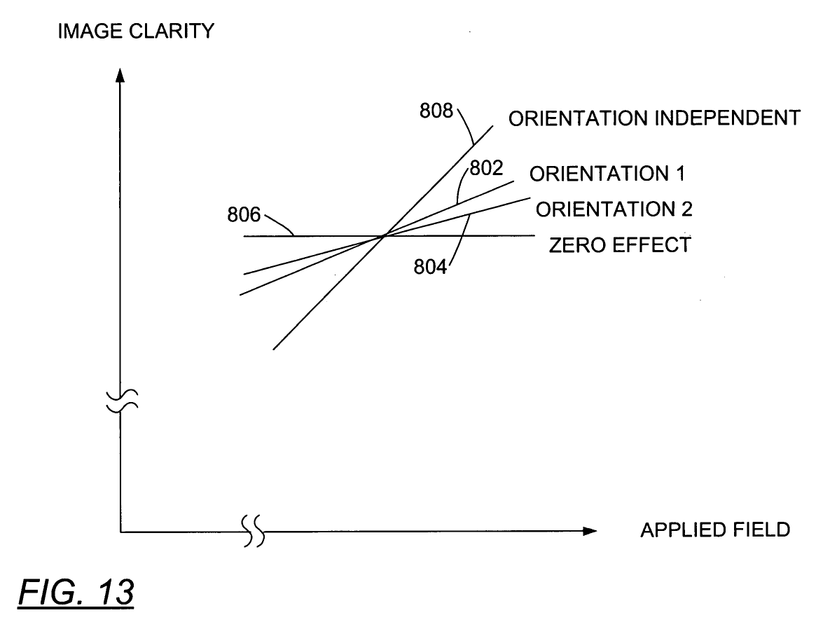

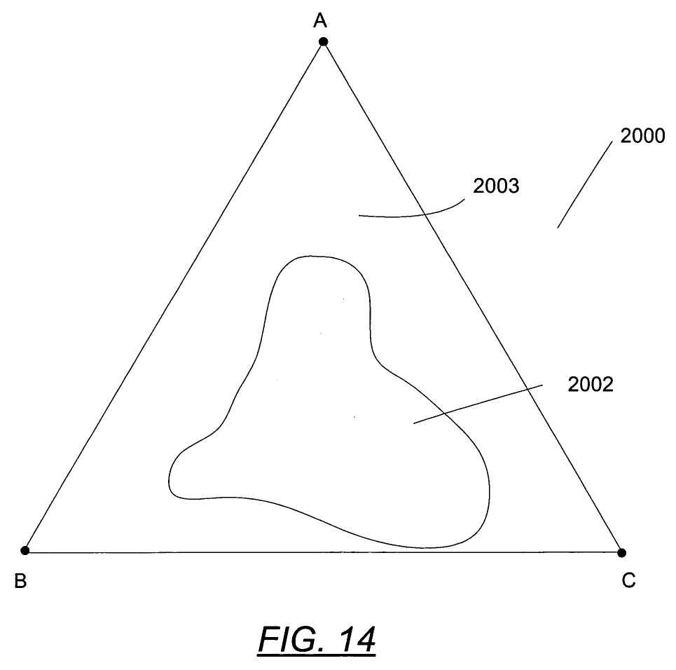

In one embodiment, the nanoelectrical particles of this invention are preferably comprised of aluminum, magnesium, and nitrogen atoms. This embodiment is illustrated in FIG. 14.

FIG. 14 illustrates a phase diagram 2000 comprised of moieties A, B, and C. Moiety A is preferably selected from the group consisting of aluminum, copper, gold, silver, and mixtures thereof. It is preferred that the moiety A have a resistivity of from about 2 to about 100 microohm-centimeters. In one preferred embodiment, A is aluminum with a resistivity of about 2.824 microohm-centimeters. As will apparent, other materials with resistivities within the desired range also may be used.

Referring again to FIG. 14, C is selected from the group consisting of nitrogen and oxygen. It is preferred that C be nitrogen, and A is aluminum; and aluminum nitride is present as a phase in system.

Referring again to FIG. 14, B is preferably a dopant that is present in a minor amount in the preferred aluminum nitride. In general, less than about 50 percent (by weight) of the B moiety is present, by total weight of the doped aluminum nitride. In one aspect of this embodiment, less than about 10 weight percent of the B moiety is present, by total weight of the doped aluminum nitride.

The B moiety may be, e.g., magnesium, zinc, tin, indium, gallium, niobium, zirconium, strontium, lanthanum, tungsten, mixtures thereof, and the like. In one embodiment, B is selected from the group consisting of magnesium, zinc, tin, and indium. In another especially preferred embodiment, the B moiety is magnesium.

Referring again to FIG. 14, and when A is aluminum, B is magnesium, and C is nitrogen, it will be seen that regions 2002 and 2003 correspond to materials which have a low relative dielectric constant (less than about 1.5), and a high relative dielectric constant (greater than about 1.5), respectively.

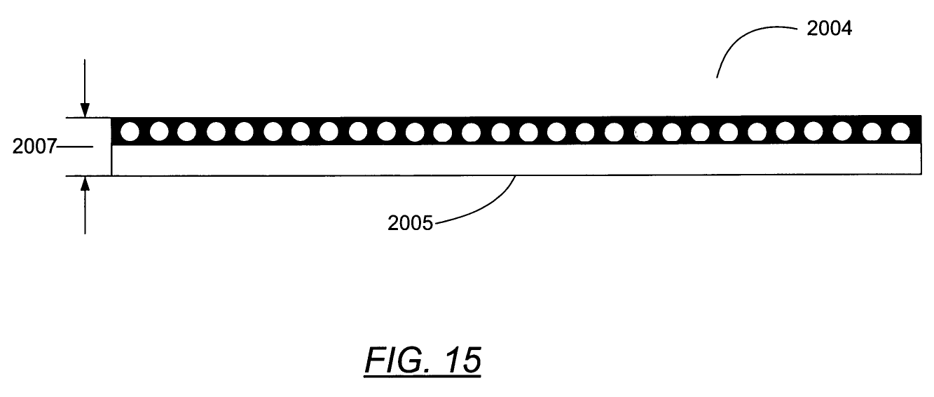

FIG. 15 is a schematic view of a coated substrate 2004 comprised of a substrate 2005 and a multiplicity of nanoelectrical particles 2006. In this embodiment, it is preferred that the nanoelectrical particles 2006 form a film with a thickness 2007 of from about 10 nanometers to about 2 micrometers and, more preferably, from about 100 nanometers to about 1 micrometer.

A Coated Substrate with a Dense Coating

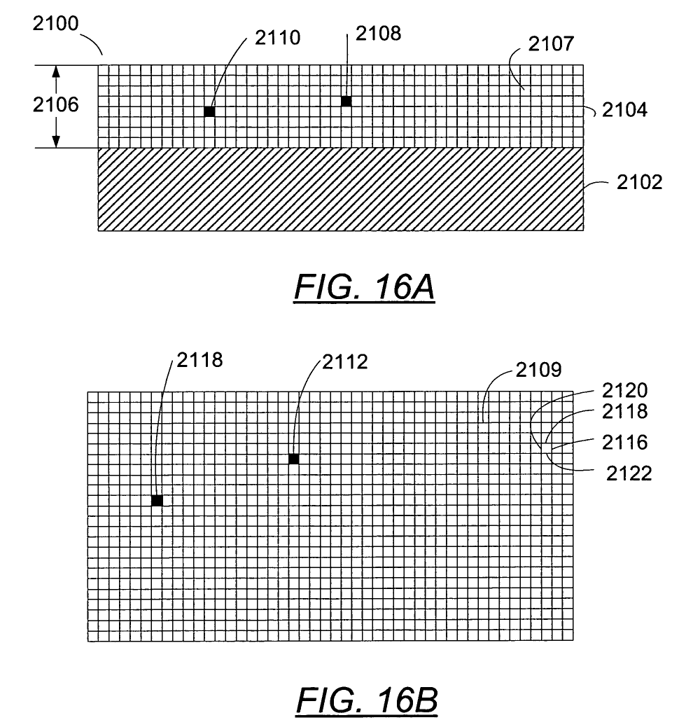

FIGS. 16A and 16B are sectional and top views, respectively, of a coated substrate 2100 assembly comprised of a substrate 2102 and, disposed therein, a coating 2104.

In the embodiment depicted, the coating 2104 has a thickness 2106 of from about 400 to about 2,000 nanometers and, in one embodiment, has a thickness of from about 600 to about 1200 nanometers.

Referring again to FIGS. 16A and 16B, it will be seen that coating 2104 has a morphological density of at least about 98 percent. As is known to those skilled in the art, the morphological density of a coating is a function of the ratio of the dense coating material on its surface to the pores on its surface; and it is usually measured by scanning electron microscopy.

By way of illustration, published United States patent application U.S. 2003/0102222A1 contains a FIG. 3A that is a scanning electron microscope (SEM) image of a coating of “long” single-walled carbon nanotubes on a substrate. Referring to this SEM image, it will be seen that the white areas are the areas of the coating where pores occur.

The technique of making morphological density measurements also is described, e.g., in a M.S. thesis by Raymond Lewis entitled “Process study of the atmospheric RF plasma deposition system for oxide coatings” that was deposited in the Scholes Library of Alfred University, Alfred, N.Y. in 1999 (call Number TP2 a75 1999 vol 1., no. 1.).

FIGS. 16A and 16B schematically illustrate the porosity of the side 2107 of coating 2104, and the top 2109 of the coating 2104. The SEM image depicted shows two pores 2108 and 2110 in the cross-sectional area 2107, and it also shows two pores 2212 and 2114 in the top 2109. As will be apparent, the SEM image can be divided into a matrix whose adjacent lines 2116/2120, and adjacent lines 2118/2122 define square portion with a surface area of 100 square nanometers (10 nanometers×10 nanometers). Each such square portion that contains a porous area is counted, as is each such square portion that contains a dense area. The ratio of dense areas/porous areas, ×100, is preferably at least 98. Put another way, the morphological density of the coating 2104 is at least 98 percent. In one embodiment, the morphological density of the coating 2104 is at least about 99 percent. In another embodiment, the morphological density of the coating 2104 is at least about 99.5 percent.

One may obtain such high morphological densities by atomic size deposition, i.e., the particles sizes deposited on the substrate are atomic scale. The atomic scale particles thus deposited often interact with each other to form nano-sized moieties that are less than 100 nanometers in size.

In one embodiment, the coating 2104 (see FIGS. 16A and 16B) has an average surface roughness of less than about 100 nanometers and, more preferably, less than about 10 nanometers. As is known to those skilled in the art, the average surface roughness of a thin film is preferably measured by an atomic force microscope (AFM). Reference may be had, e.g., to U.S. Pat. No. 5,420,796 (method of inspecting planarity of wafer surface), U.S. Pat. Nos. 6,610,004, 6,140,014, 6,548,139, 6,383,404, 6,586,322, 5,832,834, and 6,342,277. The entire disclosure of each of these United States patents is hereby incorporated by reference into this specification.

Alternatively, or additionally, one may measure surface roughness by a laser interference technique. This technique is well known. Reference may be had, e.g., to U.S. Pat. No. 6,285,456 (dimension measurement using both coherent and white light interferometers), U.S. Pat. Nos. 6,136,410, 5,843,232 (measuring deposit thickness), U.S. Pat. No. 4,151,654 (device for measuring axially symmetric aspherics), and the like. The entire disclosure of these United States patents are hereby incorporated by reference into this specification.

In one embodiment, the coated substrate of this invention has durable magnetic properties that do not vary upon extended exposure to a saline solution. If the magnetic moment of a coated substrate is measured at “time zero” (i.e., prior to the time it has been exposed to a saline solution), and then the coated substrate is then immersed in a saline solution comprised of 7.0 mole percent of sodium chloride and 93 mole percent of water, and if the substrate/saline solution is maintained at atmospheric pressure and at temperature of 98.6 degrees Fahrenheit for 6 months, the coated substrate, upon removal from the saline solution and drying, will be found to have a magnetic moment that is within plus or minus 5 percent of its magnetic moment at time zero.

In another embodiment, the coated substrate of this invention has durable mechanical properties when tested by the saline immersion test described above.

In one embodiment, the coating 2104 is biocompatible with biological organisms. As used herein, the term biocompatible refers to a coating whose chemical composition does not change substantially upon exposure to biological fluids. Thus, when the coating 2104 is immersed in a 7.0 mole percent saline solution for 6 months maintained at a temperature of 98.6 degrees Fahrenheit, its chemical composition (as measured by, e.g., energy dispersive X-ray analysis [EDS, or EDAX]) is substantially identical to its chemical composition at “time zero.”

A Preferred Process of the Invention

In one embodiment of the invention, best illustrated in FIG. 9, a coated stent is imaged by an MRI imaging process. As will be apparent to those skilled in the art, the process depicted in FIG. 9 can be used with reference to other medical devices such as, e.g., a coated brachytherapy seed (see, e.g., FIG. 1).

In the first step of this process, the coated stent described by reference to FIG. 9 is contacted with the radio-frequency, direct current, and gradient fields normally associated with MRI imaging processes; these fields are discussed elsewhere in this specification. They are depicted as an MRI imaging signal 440 in FIG. 9

In the second step of this process, the MRI imaging signal 440 penetrates the coated stent 400 and interacts with material disposed on the inside of such stent, such as, e.g., plaque particles 430 and 432. This interaction produces a signal best depicted as arrow 441 in FIG. 9.

In one embodiment, the signal 440 is substantially unaffected by its passage through the coated stent 400. Thus, in this embodiment, the radio-frequency field that is disposed on the outside of the coated stent 400 is substantially the same as the radio-frequency field that passes through and is disposed on the inside of the coated stent 400. It is preferred that at least about 90 percent of such r.f. field pass through to the inside of the coated stent 400. In such a case, the stent is said to have a radio frequency shielding factor of less than about ten percent.

By comparison, when the stent (not shown) is not coated with the coatings of this invention, the characteristics of the signal 440 are substantially varied by its passage through the uncoated stent. Thus, with such uncoated stent, the radio-frequency signal that is disposed on the outside of the stent (not shown) differs substantially from the radio-frequency field inside of the uncoated stent (not shown). In some cases, because of substrate effects, substantially none of such radio-frequency signal passes through the uncoated stent (not shown).

In the third step of this process, and in one embodiment thereof, the MRI field(s) interact with material disposed on the inside of coated stent 400 such as, e.g., plaque particles 430 and 432. This interaction produces a signal 441 by means well known to those in the MRI imaging art.

In the fourth step of the preferred process of this invention, the signal 441 passes back through the coated stent 400 in a manner such that it is substantially unaffected by the coated stent 400. Thus, in this embodiment, the radio-frequency field that is disposed on the inside of the coated stent 400 is substantially the same as the radio-frequency field that passes through and is disposed on the outside of the coated stent 400.

By comparison, when the stent (not shown) is not coated with the coatings of this invention, the characteristics of the signal 441 are substantially varied by its passage through the uncoated stent. Thus, with such uncoated stent, the radio-frequency signal that is disposed on the inside of the stent (not shown) differs substantially from the radio-frequency field outside of the uncoated stent (not shown). In some cases, because of substrate effects, substantially none of such signal 441 passes through the uncoated stent (not shown).

Another Preferred Process of the Invention

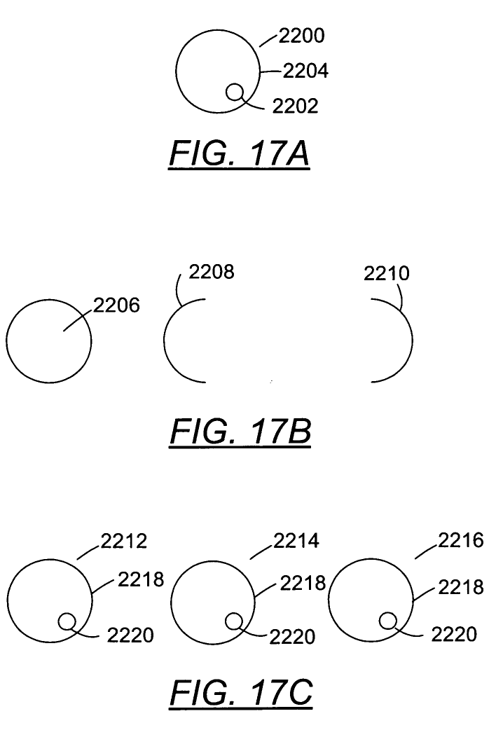

FIGS. 17A, 17B, and 17C illustrate another preferred process of the invention in which a medical device (such as, e.g., a stent 2200) may be imaged with an MRI imaging process. In the embodiment depicted in FIG. 17A, the stent 2200 is comprised of plaque 2202 disposed inside the inside wall 2204 of the stent 2200.

FIG. 17B illustrates three images produced from the imaging of stent 2200, depending upon the orientation of such stent 2200 in relation to the MRI imaging apparatus reference line (not shown). With a first orientation, an image 2206 is produced. With a second orientation, an image 2208 is produced. With a third orientation, an image 2210 is produced.

By comparison, FIG. 17C illustrates the images obtained when the stent 2200 has the nanomagnetic coating of this invention disposed about it. Thus, when the coated stent 400 of FIG. 9 is imaged, the images 2212, 2214, and 2216 are obtained.

The images 2212, 2214, and 2216 are obtained when the coated stent 400 is at the orientations of the uncoated stent 2200 the produced images 2206, 2208, and 2210, respectively. However, as will be noted, despite the variation in orientations, one obtains the same image with the coated stent 400.

Thus, e.g., the image 2218 of the coated stent (or other coated medical device) will be identical regardless of how such coated stent (or other coated medical device) is oriented vis-a-vis the MRI imaging apparatus reference line (not shown). Thus, e.g., the image 2220 of the plaque particles will be the same regardless of how such coated stent is oriented vis-a-vis the MRI imaging apparatus reference line (not shown).

Consequently, in this embodiment of the invention, one may utilize a nanomagnetic coating that, when imaged with the MRI imaging apparatus, will provide a distinctive and reproducible imaging response regardless of the orientation of the medical device.

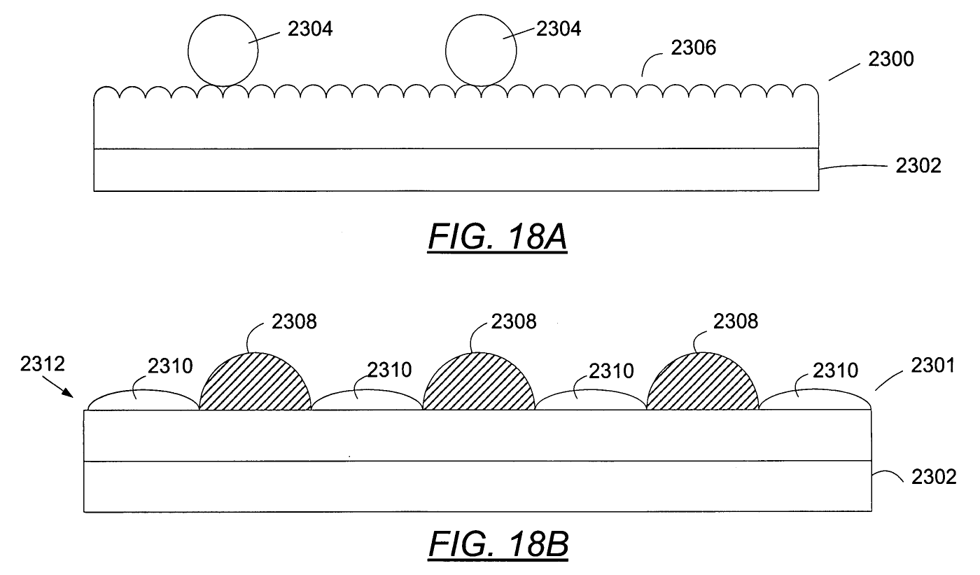

FIGS. 18A and 18B illustrate a hydrophobic coating 2300 and a hydrophilic coating 2301 that may be produced by the process of this invention.

As is known to those skilled in the art, a hydrophobic material is antagonistic to water and incapable of dissolving in water. A hydrophobic surface is illustrated in FIG. 18A.

Referring to FIG. 18A, it will be seen that a coating 2300 is deposited onto substrate 2302. In the embodiment depicted, the coating 2300 an average surface roughness of less than about 1 nanometer. Inasmuch as the average water droplet has a minimum cross-sectional dimension of at least about 3 nanometers, the water droplets 2304 will tend not to bond to the coated surface 2306 which, thus, is hydrophobic with regard to such water droplets.

One may vary the average surface roughness of coated surface 2306 by varying the pressure used in the sputtering process described elsewhere in this specification. In general, the higher the gas pressure used, the rougher the surface.

FIG. 18BB illustrates water droplets 2308 between surface features 2310 of coated surface 2312. In this embodiment, because the surface features 2310 are spaced from each other by a distance of at least about 10 nanometers, the water droplets 2308 have an opportunity to bond to the surface 2312 which, in this embodiment, is hydrophilic.

The Bond Formed Between the Substrate and the Coating

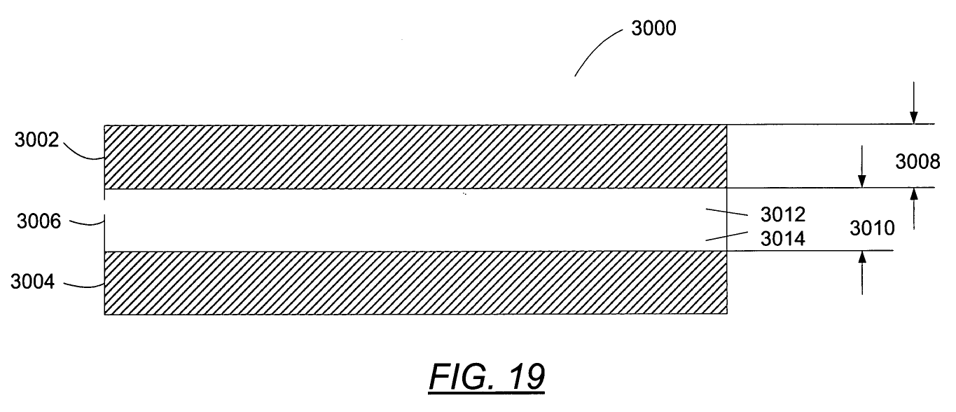

Applicants believe that, in at least one preferred embodiment of the process of their invention, the particles in their coating diffuse into the substrate being coated to form a interfacial diffusion layer. This structure is best illustrated in FIG. 19 which, as will be apparent, is not drawn to scale.

Referring to FIG. 19, the coated assembly 3000 is preferably comprised of a coating 3002 disposed on a substrate 3004. The coating 3002 preferably has at thickness 3008 of at least about 150 nanometers.

The interlayer 3006, by comparison, has a thickness of 3010 of less than about 10 nanometers and, preferably, less than about 5 nanometers. In one embodiment, the thickness of interlayer 3010 is less than about 2 nanometers.

The interlayer 3006 is preferably comprised of a heterogeneous mixture of atoms from the substrate 3004 and the coating 3002. It is preferred that at least 10 mole percent of the atoms from the coating 3002 are present in the interlayer 3006, and that at least 10 mole percent of the atoms from the substrate 3004 are in the interlayer 3006. It is more preferred that from about 40 to about 60 mole percent of the atoms from each of the coating and the substrate be present in the interlayer 3006, it being apparent that more atoms from the coating will be present in that portion 3012 of the interlayer closest to the coating, and more atoms from the substrate will be present in that portion 3014 closest to the substrate.

In one embodiment, the substrate 3004 will consist essentially of niobium atoms with from about 0 to about 2 molar percent of zirconium atoms present. In another embodiment, the substrate 3004 will comprise nickel atoms and titanium atoms. In yet another embodiment, the substrate will comprise tantalum atoms, or titanium atoms.

The coating may comprise any of the A, B, and/or C atoms described hereinabove. By way of way of illustration, the coating may comprise aluminum atoms and oxygen atoms (in the form of aluminum oxide), iridium atoms and oxygen atoms (in the form of iridium oxide), etc.

A Coated Substrate with a Specified Surface Morphology

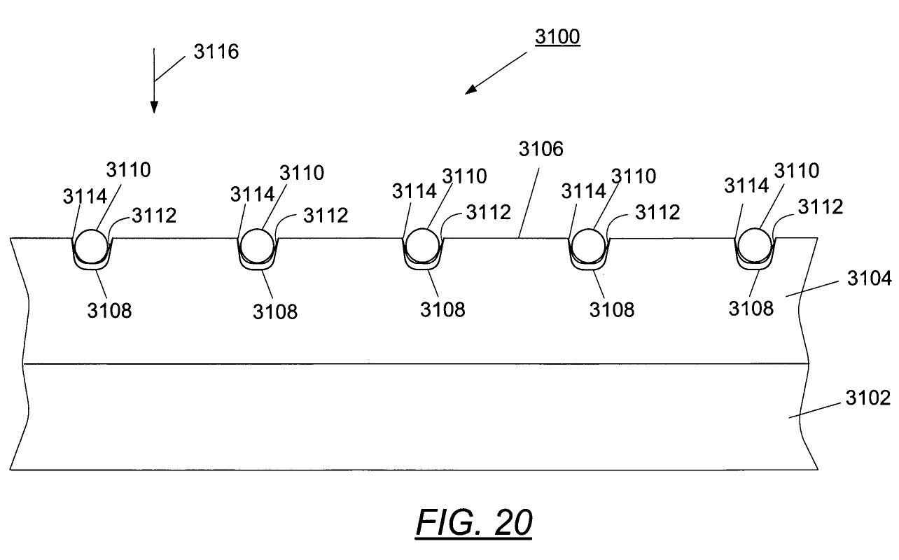

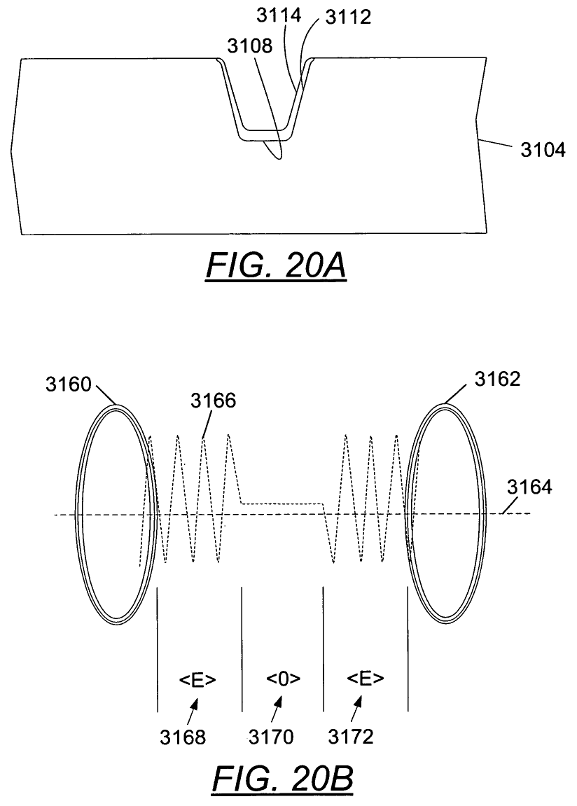

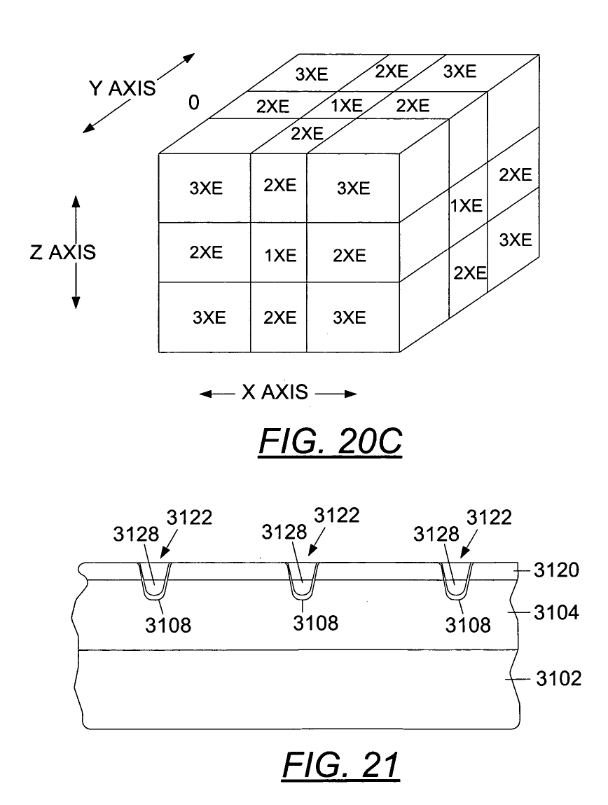

FIG. 20 is a sectional schematic view of a coated substrate 3100 comprised of a substrate 3102 and, bonded thereto, a layer 3104 of nano-sized particles that may comprise nanomagnetic particles, nanoelectrical particles, nanoinsulative particles, nanothermal particles. These particles, the mixtures thereof, and the matrices in which they are disposed have all been described elsewhere in this specification. Depending upon the properties desired from the coated substrate 3100 and/or the layer 3104, one may use one or more of the coating constructs described elsewhere in this specification. Thus, e.g., depending upon the type of particle(s) used and its properties, one may produce a desired set of electrical and magnetic properties for either the coated substrate 3100, the substrate 3200, and/or the coating 3104.

In one embodiment, the coating 3104 is comprised of at least about 5 weight percent of nanomagnetic material with the properties described elsewhere in this specification. In another embodiment, the coating 3104 is comprised of at least 10 weight percent of nanomagnetic material. In yet another embodiment, the coating 3104 is comprised of at least about 40 weight percent of nanomagnetic material.

Referring again to FIG. 20, and to the preferred embodiment depicted therein, the surface 3106 of the coating 3104 is comprised of a multiplicity of morphological indentations 3108 sized to receive drug particles 3110.

Anti-Microtubule Agents with a Magnetic Moment

In one embodiment of the process of this invention, the drug particles 3110 used (see FIG. 20) are particles of an anti-microtubule agent with a magnetic moment. Some of these “magnetic moment anti-microtubule agents” are disclosed in applicants' copending U.S. patent application U.S. Ser. No. 60/516,134, filed on Oct. 31, 2003, the entire disclosure of which is hereby incorporated by reference into this specification.” Other of these “magnetic moment anti-microtubule agents” are disclosed in applicants' copending patent application U.S. Ser. No. 10/887,521, filed on Jul. 7, 2004, the entire disclosure of which is hereby incorporated by reference into this specification In one embodiment, paclitaxel is bonded to the nanomagnetic particles of this invention in the manner described in U.S. Pat. No. 6,200,547, the entire disclosure of which is hereby incorporated by reference into this specification.

Referring again to FIG. 20 of the instant specification, and to the preferred embodiment depicted therein, the morphologically indented surface 3106 may be made by conventional means.

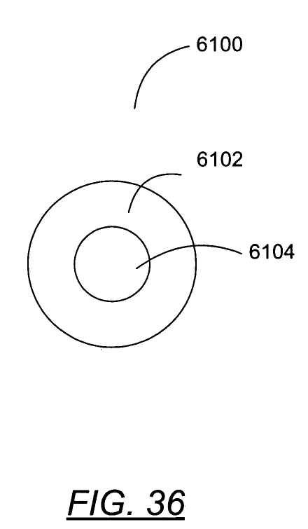

Referring again to FIG. 20, and in one preferred embodiment thereof, the size of the indentations 3108 is preferably chosen such that it matches the size of the drug particles 3110. In one embodiment, depicted in FIG. 36A, the surface 3112 of the indentations 3108 is coated with receptor material 3114 adapted to bind to the drug particles 3110.

Receptor material 3114 is comprised of a “recognition molecule”. As is known to those skilled in the art, recognition is a specific binding interaction occurring between macromolecules. These “recognition molecules” and “recognition systems” are described in copending patent application U.S. Ser. No. 10/887,521, filed on Jul. 7, 2004, the entire disclosure of which is hereby incorporated by reference into this specification Referring again to FIG. 20, and in the embodiment depicted, an external electromagnetic field 3116 is shown being applied near the surface 3106 of the coated substrate 3100. In the embodiment depicted, this applied field 3116 is adapted to facilitate the bonding of the drug particles 3110 to the indentations 3108. As long as such indentations are not totally filled, and as long as the appropriate electromagnetic field is applied, then the drug molecules 3110 will continue to bond to such indentations 3108. In one embodiment, not depicted in FIG. 20, instead of drug particles 3110 or in addition thereto, one or more of the nanomagnetic particles of this invention may be caused to bind to a specific site within a biological organism.

The external attachment electromagnetic field 3116 may, e.g., be ultrasound. It is known that ultrasound can be used to greatly enhance the rate of binding between members of a specific binding pair. Reference may be had, e.g., to U.S. Pat. No. 4,575,485, the entire disclosure of which is hereby incorporated by reference into this specification. Other ultrasound devices and processes are discussed in applicants' copending patent application U.S. Ser. No. 10/887,521, filed on Jul. 7, 2004, the entire disclosure of which is hereby incorporated by reference into this specification In one embodiment, the electromagnetic radiation used in the process of this invention is a magnetic field with a field strength of at least about 6 Tesla. It is known, e.g., that microtubules move linearly in magnetic fields of at least about 6 Tesla.

In this embodiment, the focusing of the magnetic field onto an in vivo site within a patient may be done by conventional magnetic focusing means. Some of these magnetic focusing means are disclosed in applicants' copending patent application U.S. Ser. No. 10/887,521, filed on Jul. 7, 2004, the entire disclosure of which is hereby incorporated by reference into this specification FIG. 20B is a schematic of an electromagnetic coil set 3160 and 3162, aligned to an axis 3164, and which in combination create a magnetic standing wave 3166. The excitation energy delivered to the two coils 3160 and 3162 comprises a set of high frequency sinusoidal signals that are determined via well known Fourier techniques, to create a first zone 3168 having a positive standing wave magnetic field ‘E’, a second zone 3170 having a zero or near-zero magnetic field, and a third zone 3172 having a positive magnetic field ‘E’. It should be noted that the two zones 3168 and 3172 need not have exactly matched waveforms, in frequency, phase, or amplitude; it is sufficient that the magnetic fields in both are large with respect to the near-zero magnetic field in zone 3170. The fields in zones 3168 and 3172 may be static standing wave fields or time-varying standing waves. It should be noted that in order to create a zone 3170 of useful size (1 to 5 cm at the lower limit) and having reasonably sharp ‘edges’, the frequencies of the Fourier waveforms used to create standing wave 3166 may be in the gigahertz range. These fields may be switched on and off at some secondary frequency that is substantially lower; the resulting switched-standing-wave fields in zones 3168 and 3172 will impart vibrational energy to any magnetic materials within them, while the near-zero switched field in zone 3170 will not impart substantial energy into magnetic materials within its boundaries. This secondary switching frequency may be adjusted in concert with the amplitude of the standing wave field to tune the vibrational energy to impart an optimal level of thermal energy to a specific molecule (e.g. paclitaxel) by virtue of the natural resonant frequency of that molecule. The energy imparted to an individual molecule will follow the relationship E_{T}=C×M×A×F², where E_{T} is the thermal energy imparted to an individual molecule, C is a constant, M is the magnetic moment of the molecule and any bound magnetic particles, A is the amplitude of the time-varying magnetic field, and F is the frequency of field switching.

FIG. 20C is a three-dimensional schematic showing the use of three sets of magnetic coils arranged orthogonally. Each of the axes, ‘X’, ‘Y’, and ‘Z’ will impart either positive thermal energy (E) in its outer zones that correspond to zones 3168 and 3172 (from FIG. 20B), or zero thermal energy, in its central zone which corresponds to zone 3170 (from FIG. 20B). It may be seen from FIG. 20C that there will be a small volume at the centroid of the overall 3-D volume that will have overall zero magnetically-induced thermal energy. The notations ‘1×E’, ‘2×E’, and ‘3×E’ denote the relative magnetically-induced thermal energy in other regions. Since the overall volume is made up of three zones in each of three dimensions, the overall volume will have 27 sectors. Of these sectors one (the centroid) will have near-zero magnetically-induced thermal energy, (6) sectors will have a ‘1×E’ energy level, (12) sectors will have a ‘2×E’ energy level, and (8) sectors will have a ‘3×E’ energy level.

If the energy imported to any individual molecule (e.g. paclitaxel bound to one or more nanomagnetic particles) is sufficiently larger than the binding energy of that molecule to its target (e.g. tubulin in the case of paclitaxel) to account for thermal losses in coupling magnetically-induced energy into the molecule, then binding between the paclitaxel molecule and the tubulin target will not occur. Thus if we define the binding energy between the two (e.g. paclitaxel to tubulin) as E_{B}, and D as a constant that compensates for damping losses due to a molecule that is not purely elastic, then the equation E_{T}>D×E_{B} will have been satisfied, and chemical binding (in this case between paclitaxel and tubulin) will not occur.

In one embodiment, a device having matched coil sets as shown in FIG. 20B, but in three orthogonal axes, creates an overall operational volume that imparts an relatively low energy in the above-described centroid (E_{T}<D×E_{B}), and imparts a relatively higher energy in the other surrounding (26) segments (E_{T}>D×E_{B}); and if the centroid volume corresponds to the site under treatment, then a high degree of binding will occur in the centroid and no binding will occur in the exterior regions. The size of the non-binding centroid region may be adjusted via alterations to the Fourier waveforms, relative energy levels may be adjusted via amplitude and frequency of field switching, and the region may be aligned to correspond to the volume of the tumor under treatment. One preferred method for use is to place the patient in the device as disclosed herein, administer either native paclitaxel (or other drug having an innate magnetic characteristic) or magnetically-enhanced Paclitaxel (nanomagnetic or other magnetic particles either chemically or magnetically bound), maintain the patient in the controlled fields for a period of time necessary for the drug to pass out of the patient's excretory system, and then remove the patient from the device.

In another embodiment, the three fields in the X, Y, and Z directions are selectively activated and deactivated in a predetermined pattern. For example, one may activate the field in the X axis, thus causing the therapeutic agent to align with the X axis. A certain time later the field along the X axis is deactivated and the field corresponding to the Y axis is activated for a predetermined period of time. The agent then aligns with the new axis. This may be repeated along any axis. By rapidly activating and deactivating the respective fields in a predetermined pattern, one imparts thermal and/or rotational energy to the molecule. When the energy imparted to the therapeutic agent is greater than the binding energy necessary to bring about a biological effect, such binding is drastically reduced.

In another embodiment, the Fourier techniques are selected so as to create a near-zero magnetic field zone external to the tissue to be treated, while a time-varying standing wave is generated within the centroid region. A therapeutic agent that is weakly attached to a magnetic carrier particle (a carrier-agent complex) is introduced into the body. In one embodiment, the carrier particle acts to inhibit the biological activity of the therapeutic agent. When the carrier-agent complex enters the region of variable magnetic field located at the centroid, the thermal energy imparted to the carrier-agent complex the agent is liberated from its carrier and is no longer inhibited by the presence of that carrier. The region external to the centroid is a near-zero magnetic field, thus minimizing any premature dissociation of the carrier-agent complex.

Referring again to FIGS. 20 and 36A, it will be seen that FIG. 20A is a partial sectional view of an indentation 3108 coated with a multiplicity of receptors 3114 for the drug molecules.

FIG. 21 is a schematic illustration of one process for preparing a coating with morphological indentations 3108. In this process, a mask 3120 is disposed over the film 3014. The mask 3120 is comprised of a multiplicity of holes 3122 through which etchant 3124 is applied for a time sufficient to create the desired indentations 3108

One may use conventional etching technology to prepare the desired indentations 3108. Some of these processes are disclosed in applicants' copending patent application U.S. Ser. No. 10/887,521, filed on Jul. 7, 2004, the entire disclosure of which is hereby incorporated by reference into this specification.

Referring again to FIG. 21, and to the process depicted therein, after the indentations 3108 have been formed, the etchant is removed from the holes 3122 and the indentations 3108 by conventional means, such as, e.g., by rinsing, and then receptor material 3114 is used to form the receptor surface. The receptor material 3114 may be deposited within the indentations by one or more of the techniques described elsewhere in this specification.

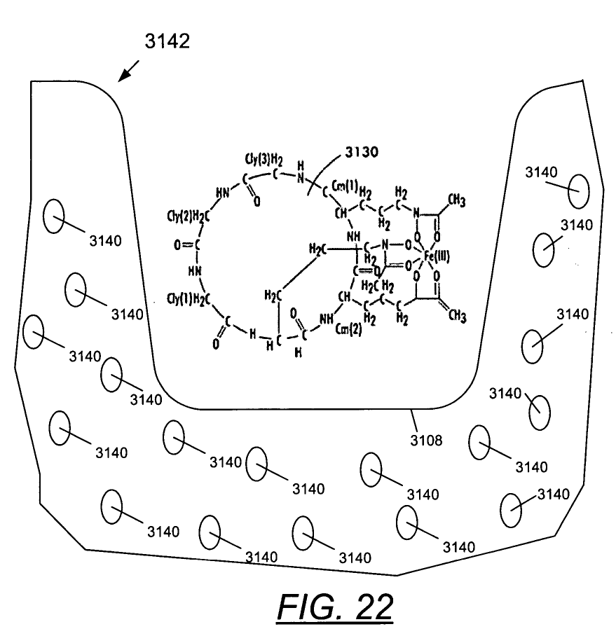

FIG. 22 is a schematic illustration of a drug molecule 3130 disposed inside of a indentation 3108. Referring to FIG. 22, and to the preferred embodiment depicted therein, it will be seen that a multiplicity of nanomagnetic particles 3140 are disposed around the drug molecule 3130. In the embodiment depicted, the forces between particles 3140 and 3130 may be altered by the application of an external field 3142. In one case, the characteristics of the field are chosen to facilitate the attachment of the particles 3130 to the particles 3140. In another case, the characteristics of the field are chosen to cause detachment of the particles 3130 from the particles 3140.

In one embodiment, the drug molecule 3130 is an anti-microtubule agent. Thus, and referring to U.S. Pat. No. 6,333,347 (the entire disclosure of which is hereby incorporated by reference into this specification), the anti-microtubule agent is preferably administered to the pericardium, heart, or coronary vasculature.

As is known to those skilled in the art, most physical and chemical interactions are facilitated by certain energy patterns, and discouraged by other energy patterns. Thus, e.g., electromagnetic attractive force may be enhanced by one applied electromagnetic filed, and electromagnetic repulsive force may be enhanced by another applied electromagnetic field. One, thus, by choosing the appropriate field(s), can determine the degree to which the one recognition molecule will bind to another, or to which a drug will bind to a implantable device, such as, e.g., a stent.

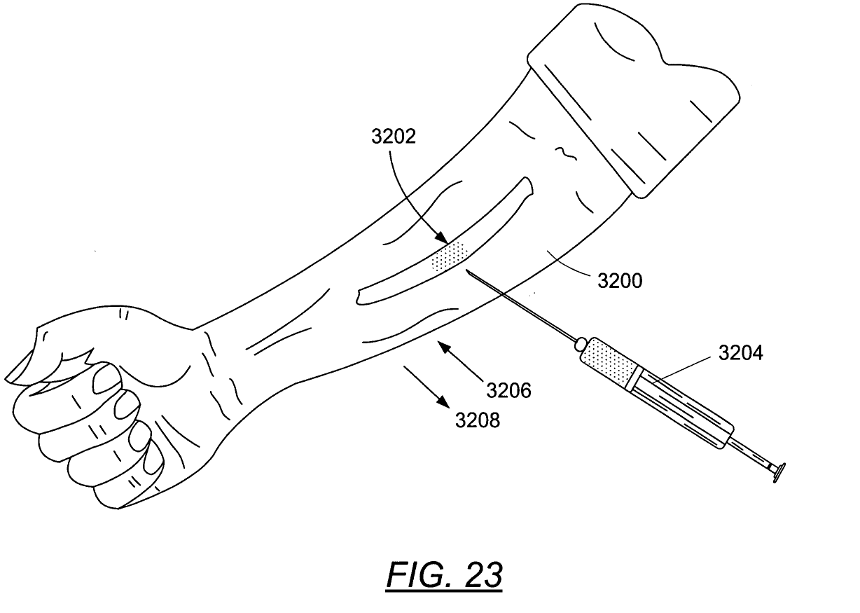

In one process, illustrated in FIG. 23, paclitaxel is administered into the arm 3200 of a patient near a stent 3202, via an injector 3204. During this administration, a first electromagnetic field 3206 is directed towards the stent 3202 in order to facilitate the binding of the paclitaxel to the stent. When it has been determined that a sufficient amount of paclitaxel has bound to the stent, a second electromagnetic field 3208 is directed towards the stent 3202 to discourage the binding of paclitaxel to the stent. The strength of the second electromagnetic field 3208 is sufficient to discourage such binding but not necessarily sufficient to dislodge paclitaxel particles already bound to the stent and disposed within indentations 3208.

A Preferred Binding Process

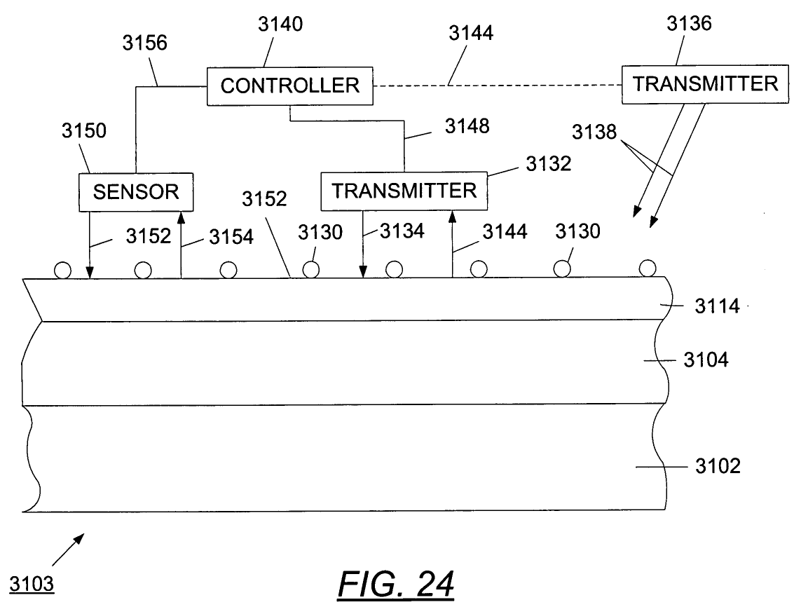

FIG. 24 is a schematic illustration of a preferred binding process of the invention.

In the first step of the process of FIG. 24, a multiplicity of drug particles, such as drug particles 3130, are brought close to or contiguous with a coated substrate 3103 comprised of receptor material 3114 disposed on its top surface. The drug particles 3130 are near and/or contiguous with the receptor material 3114. They may be delivered to such receptor material 3114 by one or more of the drug delivery processes discussed elsewhere in this specification.

In the second step of the process depicted in FIG. 24, the substrate 3102/coating 3104/receptor material 3114/drug particles 3130 assembly is contacted with electromagnetic radiation to affect, e.g., the binding of the drug particles 3130 to the receptor material 3114. This may be done by, e.g., the transmission of ultrasonic radiation, as is discussed elsewhere in this specification. Alternatively, or additionally, it may be done by the use of other electromagnetic radiation that is known to affect the rate of binding between two recognition moieties and/or other biological processes.

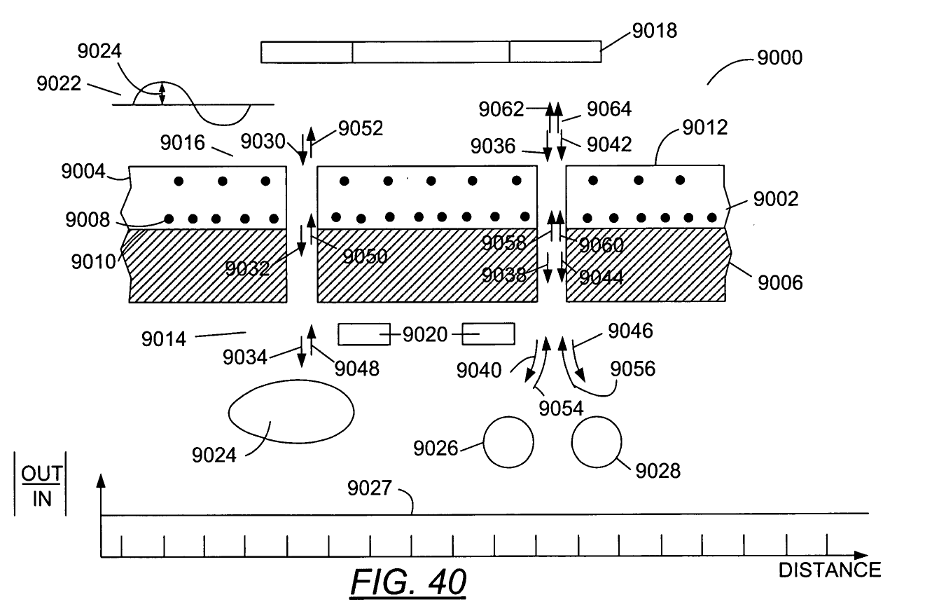

The electromagnetic radiation may be conveyed by transmitter 3132 in the direction of arrow 3134. Alternatively, or additionally, the electromagnetic radiation may be conveyed by transmitter 3136 in the direction of arrows 3138. In the embodiment depicted in FIG. 40, both transmitter 3132 and/or transmitter 3136 are operatively connected to a controller 3140. The connection may be by direct means (such as, e.g., line 3142), and/or by indirect means (such as, e.g., telemetry link 3144).

Referring again to FIG. 24, and in the preferred embodiment depicted therein, transmitter 3132 is comprised of a sensor (not shown) that can monitor the radiation 3144 retransmitted from the surface 3114 of assembly 3103.

One may use many forms of electromagnetic radiation to affect the binding of the drug moieties 3130 to the receptor surface 3114. By way of illustration, and referring to U.S. Pat. No. 6,095,148 (the entire disclosure of which is hereby incorporated by reference into this specification), the growth and differentiation of nerve cells may be affected by electrical stimulation of such cells. As is disclosed in column 1 of such patent, “Electrical charges have been found to play a role in enhancement of neurite extension in vitro and nerve regeneration in vivo. Examples of conditions that stimulate nerve regeneration include piezoelectric materials and electrets, exogenous DC electric fields, pulsed electromagnetic fields, and direct application of current across the regenerating nerve. Neurite outgrowth has been shown to be enhanced on piezoelectric materials such as poled polyvinylidinedifluoride (PVDF) (Aebischer et al., Brain Res., 436;165 (1987); and R. F. Valentini et al., Biomaterials, 13:183 (1992)) and electrets such as poled polytetrafluoroethylene (PTFE) (R. F. Valentini et al., Brain. Res. 480:300 (1989)). This effect has been attributed to the presence of transient surface charges in the material which appear when the material is subjected to minute mechanical stresses. Electromagnetic fields also have been shown to be important in neurite extension and regeneration of transected nerve ends. R. F. Valentini et al., Brain. Res., 480:300 (1989); J. M. Kerns et al., Neuroscience 40:93 (1991); M. J. Politis et al., J. Trauma, 28:1548 (1988); and B. F. Sisken et al., Brain. Res., 485:309 (1989). Surface charge density and substrate wettability have also been shown to affect nerve regeneration. Valentini et al., Brain Res., 480:300-304 (1989).”

By way of further illustration, and again referring to U.S. Pat. No. 5,566,685, extremely low frequency electromagnetic fields may be used to cause, e.g., “ . . . changes in enzyme activities . . . ” “ . . . stimulation of bone cell growth . . . ,” . . . suppression of nocturnal melatonin . . . ,” “ . . . quantative changes in transcripts . . . ,” changes in “ . . . gene expression of regenerating rate liver . . . ,” changes in “ . . . gene expression . . . ,” changes in “ . . . gene transcription . . . ,” changes in “ . . . modulation of RNA synthesis and degradation . . . ,” “ . . . alterations in protein kinase activity . . . ,” changes in “ . . . growth-related enzyme ornithine decarboxylase . . . ,” changes in embryological activity, “ . . . stimulation of experimental endochondral ossification . . . ,” “ . . . suppression of nocturnal melatonin . . . ,” changes in “ . . . human pineal gland function . . . ,” changes in “ . . . calcium binding . . . ,” etc. Reference may be had, in particular, to columns 2 and 3 of U.S. Pat. No. 5,566,685.

Referring again to FIG. 24, and to the preferred embodiment depicted therein, the transmitter 3132 preferably has a sensor to determine the extent to which radiation incident upon, e.g., surface 3146 is reflected. Information from transmitter 3132 may be conveyed to and from controller 3140 via line 3148.

In the embodiment depicted in FIG. 24, a sensor 3150 is adapted to sense the degree of binding on surface 3146 between the drug molecules 3130 and the receptor molecules 3114. This sensor 3150 preferably transmits radiation in the direction of arrow 3152 and senses reflected radiation traveling in the direction of arrow 3154. Information from and to controller 3140 is fed to and from sensor 3150 via line 3156.

There are many sensors known to those skilled in the art which can determine the extent to which two recognition molecules have bound to each other. Some of these sensors are disclosed in applicants' copending patent application U.S. Ser. No. 10/887,521, filed on Jul. 7, 2004, the entire disclosure of which is hereby incorporated by reference into this specification.

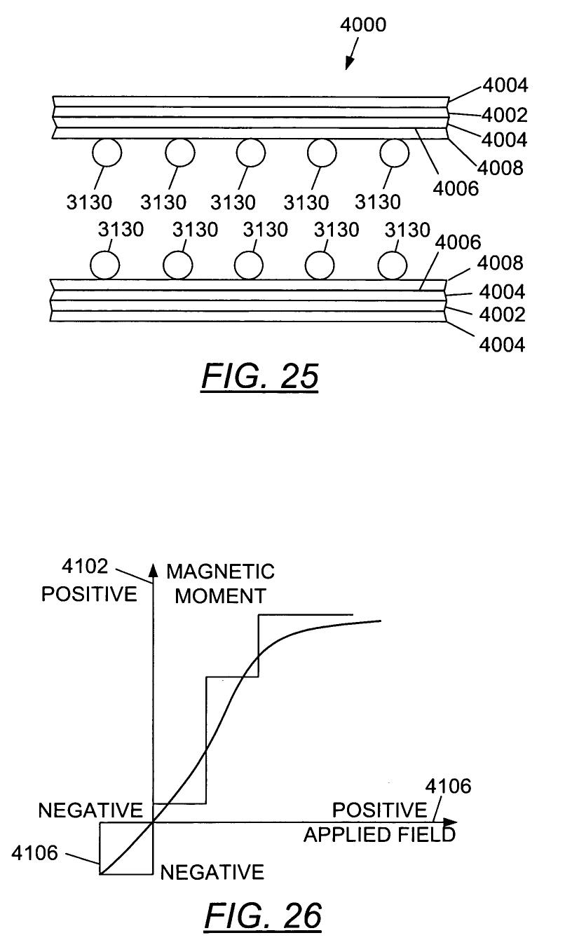

FIG. 25 is a schematic view of a preferred coated stent 4000 of the invention; as will be apparent, other coated medical devices may also be used. Referring to FIG. 25, and to the preferred embodiment depicted therein, it will be seen that coated stent 4000 is comprised of a stent 4002 onto which is deposited one or more of the nanomagnetic coatings 4004 described elsewhere in this specification. Disposed above the nanomagnetic coatings 4004 is a coating of drug-eluting polymer 4006.

One may use any of the drug eluting polymers known to those skilled in the art to produce coated stent 4000. Alternatively, or additionally, one may use one or more of the polymeric materials 14 described elsewhere in this specification. Many of these drug-eluting polymeric compositions are disclosed in applicants' copending patent application U.S. Ser. No. 10/887,521, filed on Jul. 7, 2004, the entire disclosure of which is hereby incorporated by reference into this specification

Referring again to FIG. 25, and to the preferred embodiment depicted therein, disposed on the surface 4008 of the drug eluting polymer are a multiplicity of magnetic drug particles, such the magnetic drug particle 3130 (see FIG. 22).

FIG. 26 is a graph of a typical response of a magnetic drug particle, such as magnetic drug particles 3130 (see, e.g., FIG. 22) to an applied electromagnetic field. As will be seen by reference to FIG. 26, as the magnetic field strength 4100 of an applied magnetic field is increased along the positive axis, the magnetic moment 4102 of the magnetic drug particle(s) also continuously increases along the positive axis. As will be apparent, a decrease in the magnetic field strength also causes a decrease in magnetic moment. Thus, when the polarity of the applied magnetic field changes (see section 4106 of the graph), the magnetic moment also decreases. Thus, one may affect the magnetic moment of the magnetic drug particles by varying either the intensity of the applied electromagnetic field and/or its polarity.

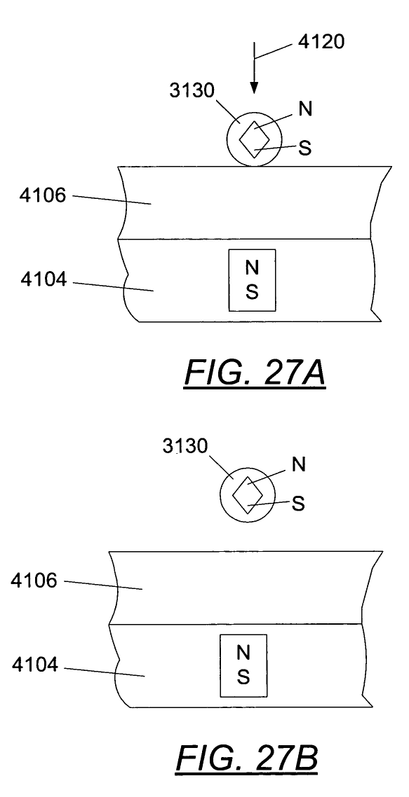

FIGS. 27A and 27B illustrate the effect of applied fields upon the nanomagnetic coating 4004 (see FIG. 25) and the magnetic drug particles 3130. Referring to FIG. 27A, when the applied magnetic field 4120 is sufficient to align the drug particle 3130 in a north(up)/south(down) orientation (see FIG. 27A), it will also tend to align the nanomagnetic material is such an orientation. However, because the magnetic hardness of the nanomagnetic material will be chosen to substantially exceed the magnetic hardness of the drug particles 3130, then the applied magnetic field will not be able to realign the nanomagnetic material.

In the ensuing discussion relating to the effects of an applied electromagnetic field, certain terms (such as, e.g., “magnetization saturation”) will be used. These terms (and others) have the meaning set forth in several of applicants' published patent applications and patents, including (without limitation) published patent application U.S. 20030107463, U.S. Pat. Nos. 6,700,472, 6,673,999, 6,506,972, 5,540,959, and the like. The entire disclosure of each of these documents is hereby incorporated by reference into this specification.

Thus, by way of illustration, reference is made to the term “magnetization.” As is disclosed in applicants' publications, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

Thus, by way of further illustration, reference is made to the term “saturation magnetization.” As is disclosed in applicants' publications, for a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification. As will be apparent to those skilled in the art, especially upon studying the aforementioned patents, the saturation magnetization of thin films is often higher than the saturation magnetization of bulk objects.

By way of further illustration, reference is made to the term “coercive force.” As is disclosed in applicants' publications, the term coercive force refers to the magnetic field, H, which must be applied to a magnetic material in a symmetrical, cyclically magnetized fashion, to make the magnetic induction, B, vanish; this term often is referred to as magnetic coercive force. Reference may be had, e.g., to U.S. Pat. Nos. 4,061,824, 6,257,512, 5,967,223, 4,939,610, 4,741,953, and the like. The entire disclosure of each of these United States patents is hereby incorporated by reference into this specification.

In one embodiment, the nanomagnetic material 103 has a coercive force of from about 0.01 to about 3,000 Oersteds. In yet another embodiment, the nanomagnetic material 103 has a coercive force of from about 0.1 to about 10.

By way of yet further illustration, reference is made to the term relative magnetic permeability. As is disclosed in applicants' publications, the term relative magnetic permeability is equal to B/H, and is also equal to the slope of a section of the magnetization curve of the film. Reference may be had, e.g., to page 4-28 of E. U. Condon et al.'s “Handbook of Physics” (McGraw-Hill Book Company, Inc., New York, 1958). Reference also may be had to page 1399 of Sybil P. Parker's “McGraw-Hill Dictionary of Scientific and Technical Terms,” Fourth Edition (McGraw Hill Book Company, New York, 1989). As is disclosed on this page 1399, permeability is “ . . . a factor, characteristic of a material, that is proportional to the magnetic induction produced in a material divided by the magnetic field strength; it is a tensor when these quantities are not parallel. Reference also may be had, e.g., to U.S. Pat. Nos. 6,181,232, 5,581,224, 5,506,559, 4,246,586, 6,390,443, and the like. The entire disclosure of each of these United States patents is hereby incorporated by reference into this specification.

Referring again to FIG. 27, and in the preferred embodiment depicted therein, the magnetic hardness of the nanomagnetic material 4104 is preferably at least about 10 times as great as the magnetic hardness of the drug particles 3130. The term “magnetic hardness” is well known to those skilled in the art. Reference may be had, e.g., to the claims and specifications of U.S. Pat. Nos. 6,201,390, 5,595,454, 5,451,162, 6,534,984, 4,967,078, 3,802,854, and the like. The entire disclosure of each of these United States patents is hereby incorporated by reference into this specification.

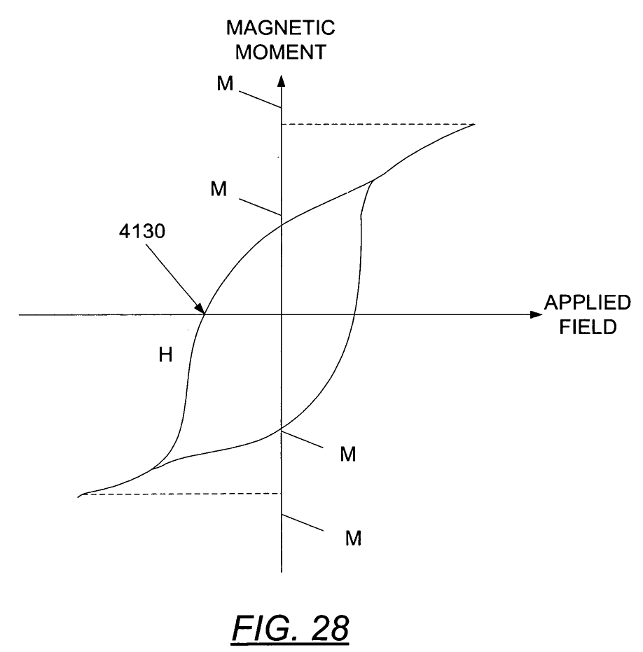

FIG. 28 is graph of a preferred nanomagnetic material and its response to an applied electromagnetic field, in which the applied field is applied against the magnetic moment of the nanomagnetic material.

As will be apparent from this FIG. 28, a certain amount of the applied electromagnetic force is required to overcome the remnant magnetization (Mr) and to change the direction of the remnant magnetization from +Mr to −Mr. Thus, e.g., the point −Hc, at point 4130, indicates how much of the field is required to make the magnetic moment be zero.

Referring again to FIGS. 27A and 27B, and in the preferred embodiments depicted therein, the Hc values of the nanomagnetic material chosen will be sufficient to realign to magnetic drug particles 3130 but insufficient to realign the nanomagnetic material. The resulting situation is depicted in FIGS. 27A and 27B.

In FIG. 27A, with the appropriate applied magnetic field, the magnetic drug particle 3130 is attached to the nanomagnetic material 4104 and thus will tend to diffuse into the polymer 4106. By comparison, in the situation depicted in FIG. 27B, the magnetic drug particles will be repelled by the nanomagnetic material. Thus, and as will be apparent, by the appropriate choice of the applied magnetic field, one can cause the magnetic drug particles either to be attracted to the layer of polymer material 4106 or to be repelled therefrom.

FIG. 29 illustrates the forces acting upon a magnetic drug particle 3130 as it approaches the nanomagnetic material 4104. Referring to FIG. 29, and in the preferred embodiment depicted therein, a certain hydrodynamic force 4140 will be applied to the particle 3130 due to the force of flow of bodily fluid, such as blood. Simultaneously, a certain attractive force 4142 will be created by the attraction of the nanomagnetic material 4104 and the particle 3130. The resulting force vector 4144 will tend to be the direction the particle 3130 will travel in. If the surface of the polymeric material is preferably comprised of a multiplicity of pores 4146, the entry of the drug particles 3130 will be facilitated into such pores.

FIG. 30 illustrates the situation that occurs after the drug particles 3130 have migrated into the layer of polymeric material and when one desires to release such drug particles. In this situation (see FIG. 27B), the applied magnetic field will be chosen such that the nanomagnetic material will tend to repel the drug particles 3130 and cause their departure into bodily fluid in the direction of arrow 4148.

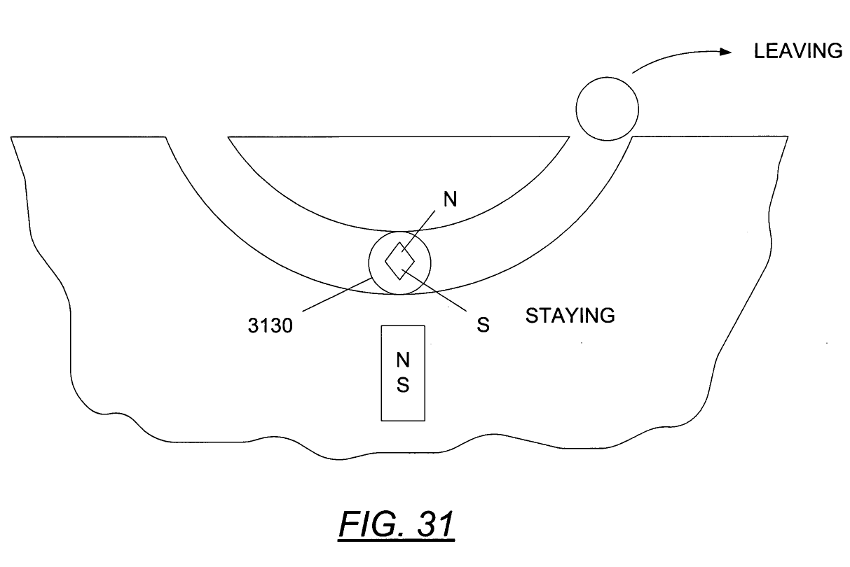

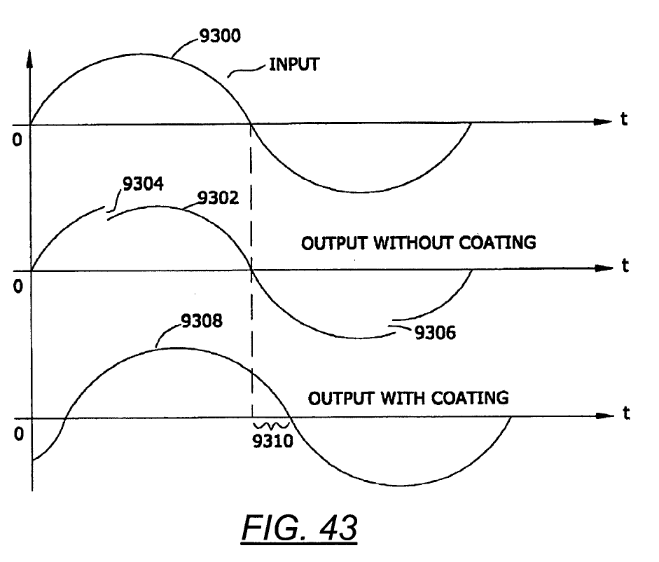

FIG. 31 illustrates the situation that occurs after the drug particles 3130 have migrated into the layer of polymeric material 4106 but when no external electromagnetic field is imposed. In this situation, there will still be an attraction between the nanomagnetic material 4104 and the magnetic drug particles 3130 that will be sufficient to keep such particles bound. However, the attraction will be weak enough such that, when hydrodynamic force 4140 is applied (see FIG. 45), the particles 3130 will elute into the bodily fluid (not shown). As will be apparent, the degree of elution in this case is less than the degree of elution in the case depicted in FIG. 43B. Thus, by the appropriate choice of electromagnetic field 4120, one can control the rate of deposition of the drug particles 3130 onto the polymer 4106, or from the polymer 4106.

Magnetic Drug Compositions

In this section of the specification, applicants will describe certain magnetic drug compositions 3130 that may be used in their preferred process. Each of these drug compositions preferably is comprised of at least one therapeutic agent and has a magnetic moment so that it can be attracted to or repelled from the nanomagnetic coatings upon application of an external electromagnetic field.

Many of these magnetic drug compositions 3130 are disclosed in applicants' copending patent application U.S. Ser. No. 10/887,521, filed on Jul. 7, 2004, the entire disclosure of which is hereby incorporated by reference into this specification

In one embodiment of the instant invention, an anti-microtubule agent (such as, e.g., paclitaxel), is adsorbed onto the surfaces of the nanoparticles. In one aspect of this embodiment, the release rate of the paclitaxel is varied by cross-linking the carbohydrate matrix after crystallization. Reference may be had, e.g., to column 4 of U.S. Pat. No. 4,501,726, the entire disclosure of which is hereby incorporated by reference into this specification.

In one embodiment, the coercive force and the remnant magnetization of applicants' nanomagnetic particles are preferably adjusted to optimize the magnetic responsiveness of the particles so that the coercive force is preferably from about 1 Gauss to about 1 Tesla and, more preferably, from about 1 to about 100 Gauss.

In one embodiment of this invention, an anti-microtubule agent (such as, e.g., paclitaxel) is incorporated into the vesicle of U.S. Pat. No. 4,652,257 and delivered to the situs of an implantable medical device, wherein the paclitaxel is released at a controlled release rate. Such a situs might be, e.g., the interior surface of a stent wherein the paclitaxel, as it is slowly released, will inhibit restenosis of the stent.

The Use of Externally Applied Energy to Affect an Implanted Medical Device

The prior art discloses many devices in which an externally applied electromagnetic field (i.e., a field originating outside of a biological organism, such as a human body) is generated in order to influence one or more implantable devices disposed within the biological organism. Some of these devices are disclosed in applicants' copending patent application U.S. Ser. No. 10/887,521, filed on Jul. 7, 2004, the entire disclosure of which is hereby incorporated by reference into this specification.

Other Compositions Comprised of Nanomagnetic Particles

In addition to the compositions already mentioned in this specification, other compositions may advantageous incorporate the nanomagnetic material of this invention. Thus, by way of illustration and not limitation, one may replace the magnetic particles in prior art compositions with the nanomagnetic materials of this invention.

In many of the prior art patents, the term “comprising magnetic particles” appears in the claims; some of these patents are disclosed in applicants' copending patent application U.S. Ser. No. 10/887,521, filed on Jul. 7, 2004, the entire disclosure of which is hereby incorporated by reference into this specification.

By way of yet further illustration, one may replace “magnetic particles” described in the medical device claimed in published U.S. patent application 2004/0030379 with applicants' nanomagnetic particles. The entire disclosure of published United States patent application U.S. 2004/0030379 is hereby incorporated by reference into this specification.

A Preferred Container Coated with Magnetostrictive Material

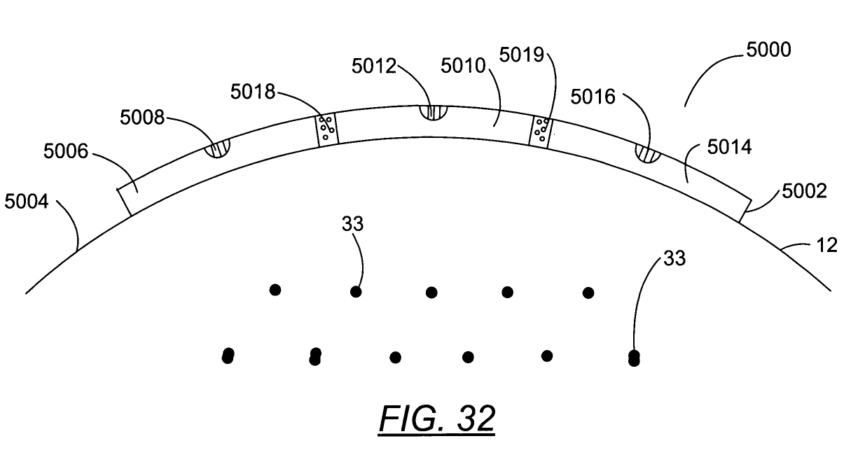

FIG. 32 is a partial view of a coated container 5000 comprised of a container 12 (see FIG. 1) over which is disposed a layer 5002 of material which changes its dimensions in response to an applied magnetic field. The material may be, e.g., magnetostrictive material, and/or it may be electrostrictive material. The direct current susceptibility of coated container 5000 is equal to the (mass of layer 5002)×(the susceptibility of layer 5002)+(the mass of container 12)×(the susceptibility of container 12). Referring again to FIG. 32, and to the preferred embodiment depicted therein, in one aspect of such embodiment the magnetostrictive materials 5006, 5010, and 5014 do not have uniform properties.

Referring again to FIG. 32, and to the preferred embodiment depicted therein, preferably disposed on the outer surface 5004 of the container 12, is a multiplicity of coatings, including a first coating of magnetostrictive material 5006 in which is disposed a first drug eluting polymer 5008, a second coating of magnetostrictive material 5010 in which is disposed a second drug eluting polymer 5012, and a third coating of magnetostrictive material 5014 in which is disposed a third drug eluting polymer 5016.

Referring again to FIG. 32, disposed between coatings 5006 and 5008 is 5018 of nanomagnetic material; and disposed between 5008 from 5010 is nanomagnetic material 5019.

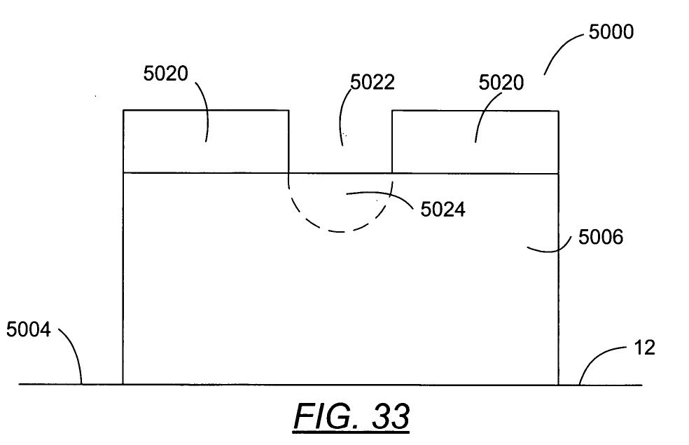

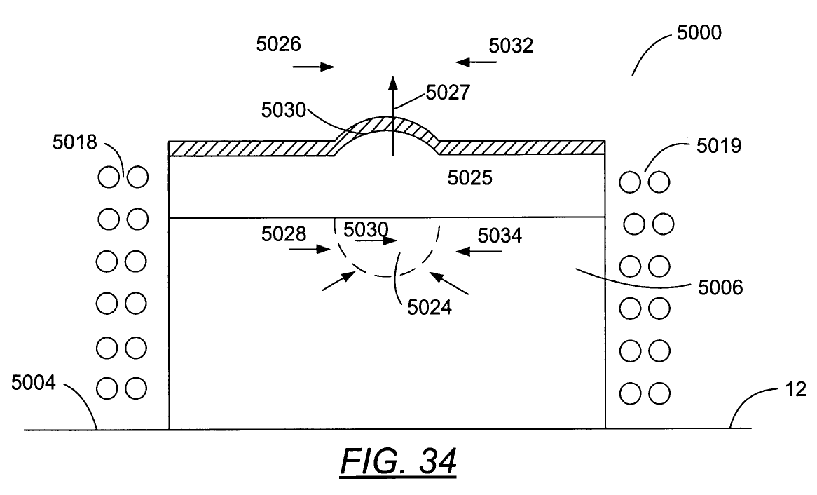

FIG. 33 is a partial view of magnetostrictive magnetostrictive material 5006 prior to the time an orifice has been created in it. In the embodiment depicted, a mask 5020 with an opening 5022 is disposed on top of the magnetostrictive material 5006, and an etchant (not shown) is disposed in said opening 5022 to create an orifice 5024, shown in dotted line outline. Thereafter, a drug-eluting polymer (such as, e.g., polymer 5008) is contacted with said etched surface and disposed within the orifice 5024. The resulting structure is shown in FIG. 34.

FIG. 34 shows the magnetostrictive material 50065 bounded by nanomagnetic material 5018/5019, and it illustrates how such assembly responds when the magnetostrictive material is subjected to one or more magnetic fields adapted to cause distortion of the material.

In the embodiment depicted in FIG. 34, a first direct current magnetic field 5026 causes force to act in the direction of arrow 5028, thereby causing distortion of the polymeric material 5024 in the direction of arrow 5030. When a second varying magnetic field 5032 (nominal direction) is applied, it causes force to act in the direction of arrow 5034. These fields, and others, may act simultaneously or sequentially to pump the material 5025 within orifice 5024 out of such orifice. The material 5025, in one embodiment, is caused to move in the direction of arrow 5027, to cause a layer of material 5029 (which may be the same as or different than material 5025) to distend, and to thus rupture pressure rupturable seal 5030.

An Implantable Medical Device with Minimal Susceptibility

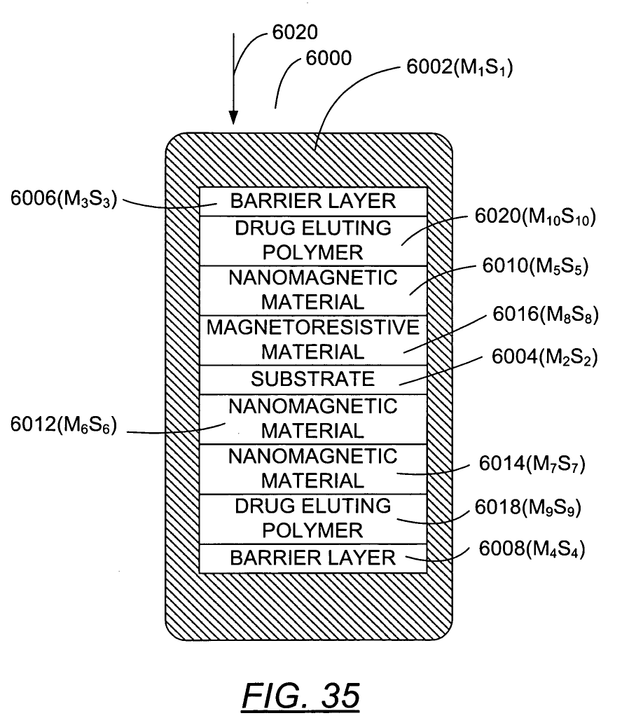

FIG. 35 presents a solution to a problem posed in published U.S. patent application 2004/0030379, the entire disclosure of which is hereby incorporated by reference into this specification and which is discussed elsewhere in this specification. This published patent application discloses (at page 1 thereof) that: “In the medical field, magnetic resonance imaging (MRI) is used to non-invasively produce medical information. The patient is positioned in an aperture of a large annular magnet, and the magnet produces a strong and static magnetic field, which forces hydrogen and other chemical elements in the patient's body into alignment with the static field. A series of radio frequency (RF) pulses are applied orthogonally to the static magnetic field at the resonant frequency of one of the chemical elements, such as hydrogen in the water in the patient's body. The RF pulses force the spin of protons of chemical elements, such as hydrogen, from their magnetically aligned positions and cause the electrons to precess. This precession is sensed to produce electromagnetic signals that are used to create images of the patient's body. In order to create an image of a plane of patient cross-section, pulsed magnetic fields are superimposed on the high strength static magnetic field.” Published United States patent application U.S. 2004/0093075 also discloses that: “While researching heart problems, it was found that all the currently used metal stents distorted the magnetic resonance images of blood vessels. As a result, it was impossible to study the blood flow in the stents and the area directly around the stents for determining tissue response to different stents in the heart region.

Published U.S. patent application 2004/0093075 also discloses that: “A solution, which would allow the development of a heart valve which could be inserted with the patients only slightly sedated, locally anesthetized, and released from the hospital quickly (within a day) after a procedure and would allow the in situ magnetic resonance imaging of stents, has long been sought but yet equally as long eluded those skilled in the art.” Such a solution is disclosed in FIG. 35 of the instant application.

The device 6000 depicted in FIG. 35, in one embodiment, is an assembly comprised of a device and material within which such device is disposed, wherein the direct current magnetic susceptibility of such assembly is plus or minus 1×10^{-3.}

Referring to FIG. 35, there is disclosed an assembly 6000 comprised of a first material 6002 (with a first mass [M₁]and a first magnetic susceptibility [S₁]) that, in the embodiment depicted, is contiguous with a substrate 6004 (with a second mass [M₂] and a second magnetic susceptibility [S2]).

In one preferred embodiment, the substrate 6004 is an implantable medical device. Thus, and as is disclosed in published U.S. patent application 2004/0030379 (the entire disclosure of which is hereby incorporated by reference into this specification), the implanted medical device may be a stent. Thus, and referring to page 4 of such published patent application, “Medical devices which are particularly suitable for the present invention include any kind of stent for medical purposes, which are known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. A bifurcated stent is also included among the medical devices suitable for the present invention.”

As is also disclosed in published U.S. patent application 2004/0030379. “The medical devices suitable for the present invention may be fabricated from polymeric and/or metallic materials. Examples of such polymeric materials include polyurethane and its copolymers, silicone and its copolymers, ethylene vinyl-acetate, poly(ethylene terephthalate), thermoplastic elastomer, polyvinyl chloride, polyolephines, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen and chitins. Examples of suitable metallic materials include metals and alloys based on titanium (e.g., nitinol, nickel titanium alloys, thermo-memory alloy materials), stainless steel, platinum, tantalum, nickel-chrome, certain cobalt alloys including cobalt-chromium-nickel alloys (e.g., Elgiloy® and Phynox®) and gold/platinum alloy. Metallic materials also include clad composite filaments, such as those disclosed in WO 94/16646.”

In one preferred embodiment, the substrate 6004 is a conventional drug-eluting medical device (such as, e.g., a drug eluting stent) to which the nanomagnetic material of this invention has been added as described hereinbelow. One may use, and modify, any of the prior art self-eluting medical devices.

By way of illustration, and as is disclosed in U.S. Pat. Nos. 5,591,227, 5,599,352, and 6,597,967 (the entire disclosure of each of which is hereby incorporated by reference into this specification), the medical device may be “ . . . a drug eluting intravascular stent comprising: (a) a generally cylindrical stent body; (b) a solid composite of a polymer and a therapeutic substance in an adherent layer on the stent body; and (c) fibrin in an adherent layer on the composite.” In the device of U.S. Pat. No. 5,591,227, the fibrin was used to provide a biocompatible surface. In the device 6000 depicted in FIG. 35, it may be used as, or in place of barrier layer 6006 and/or barrier layer 6008.

By way of yet further illustration, and as is disclosed in U.S. Pat. No. 6,623,521 (the entire disclosure of which is hereby incorporated by reference into this specification), the medical device may be an expandable stent with sliding and locking radial elements. This patent discloses many “prior art” stents, whose designs also may be modified by the inclusion of nanomagnetic material. Thus as is disclosed at columns 1-2 of this patent, “Examples of prior developed stents have been described by Balcon et al., “Recommendations on Stent Manufacture, Implantation and Utilization,” European Heart Journal (1997), vol. 18, pages 1536-1547, and Phillips, et al., “The Stenter's Notebook,” Physician's Press (1998), Birmingham, Mich. The first stent used clinically was the self-expanding “Wallstent” which comprised a metallic mesh in the form of a Chinese fingercuff. This design concept serves as the basis for many stents used today. These stents were cut from elongated tubes of wire braid and, accordingly, had the disadvantage that metal prongs from the cutting process remained at the longitudinal ends thereof. A second disadvantage is the inherent rigidity of the cobalt based alloy with a platinum core used to form the stent, which together with the terminal prongs, makes navigation of the blood vessels to the locus of the lesion difficult as well as risky from the standpoint of injury to healthy tissue along the passage to the target vessel. Another disadvantage is that the continuous stresses from blood flow and cardiac muscle activity create significant risks of thrombosis and damage to the vessel walls adjacent to the lesion, leading to restenosis. A major disadvantage of these types of stents is that their radial expansion is associated with significant shortening in their length, resulting in unpredictable longitudinal coverage when fully deployed.”

As is also disclosed in U.S. Pat. No. 6,623,521 “Among subsequent designs, some of the most popular have been the Palmaz-Schatz slotted tube stents. Originally, the Palmaz-Schatz stents consisted of slotted stainless steel tubes comprising separate segments connected with articulations. Later designs incorporated spiral articulation for improved flexibility. These stents are delivered to the affected area by means of a balloon catheter, and are then expanded to the proper size. The disadvantage of the Palmaz-Schatz designs and similar variations is that they exhibit moderate longitudinal shortening upon expansion, with some decrease in diameter, or recoil, after deployment. Furthermore, the expanded metal mesh is associated with relatively jagged terminal prongs, which increase the risk of thrombosis and/or restenosis. This design is considered current state of the art, even though their thickness is 0.004 to 0.006 inches.”

As is also disclosed in U.S. Pat. No. 6,623,521, “Another type of stent involves a tube formed of a single strand of tantalum wire, wound in a sinusoidal helix; these are known as coil stents. They exhibit increased flexibility compared to the Palnaz-Schatz stents. However, they have the disadvantage of not providing sufficient scaffolding support for many applications, including calcified or bulky vascular lesions. Further, the coil stents also exhibit recoil after radial expansion.”

As is also disclosed in U.S. Pat. No. 6,623,521, “One stent design described by Fordenbacher, employs a plurality of elongated parallel stent components, each having a longitudinal backbone with a plurality of opposing circumferential elements or fingers. The circumferential elements from one stent component weave into paired slots in the longitudinal backbone of an adjacent stent component. By incorporating locking means within the slotted articulation, the Fordenbacher stent may minimize recoil after radial expansion. In addition, sufficient numbers of circumferential elements in the Fordenbacher stent may provide adequate scaffolding. Unfortunately, the free ends of the circumferential elements, protruding through the paired slots, may pose significant risks of thrombosis and/or restenosis. Moreover, this stent design would tend to be rather inflexible as a result of the plurality of longitudinal backbones.”

As is also disclosed in U.S. Pat. No. 6,623,521, “Some stents employ “jelly roll” designs, wherein a sheet is rolled upon itself with a high degree of overlap in the collapsed state and a decreasing overlap as the stent unrolls to an expanded state. Examples of such designs are described in U.S. Pat. No. 5,421,955 to Lau, U.S. Pat. Nos. 5,441,515 and 5,618,299 to Khosravi, and U.S. Pat. No. 5,443,500 to Sigwart. The disadvantage of these designs is that they tend to exhibit very poor longitudinal flexibility. In a modified design that exhibits improved longitudinal flexibility, multiple short rolls are coupled longitudinally. See e.g., U.S. Pat. No. 5,649,977 to Campbell and U.S. Pat. Nos. 5,643,314 and 5,735,872 to Carpenter. However, these coupled rolls lack vessel support between adjacent rolls.”

As is also disclosed in U.S. Pat. No. 6,623,521, “Another form of metal stent is a heat expandable device using Nitinol or a tin-coated, heat expandable coil. This type of stent is delivered to the affected area on a catheter capable of receiving heated fluids. Once properly situated, heated saline is passed through the portion of the catheter on which the stent is located, causing the stent to expand. The disadvantages associated with this stent design are numerous. Difficulties that have been encountered with this device include difficulty in obtaining reliable expansion, and difficulties in maintaining the stent in its expanded state.”

As is also disclosed in U.S. Pat. No. 6,623,521, “Self-expanding stents are also available. These are delivered while restrained within a sleeve (or other restraining mechanism), that when removed allows the stent to expand. Self-expanding stents are problematic in that exact sizing, within 0.1 to 0.2 mm expanded diameter, is necessary to adequately reduce restenosis. However, self-expanding stents are currently available only in 0.5 mm increments. Thus, greater selection and adaptability in expanded size is needed.”

The stent design claimed in U.S. Pat. No. 6,623,521 is: An expandable intraluminal stent, comprising: a tubular member comprising a clear through-lumen, and having proximal and distal ends and a longitudinal length defined there between, a circumference, and a diameter which is adjustable between at least a first collapsed diameter and at least a second expanded diameter, said tubular member comprising: at least one module comprising a series of radial elements, wherein each radial element defines a portion of the circumference of the tubular member and wherein no radial element overlaps with itself in either the first collapsed diameter or the second expanded diameter; at least one articulating mechanism which permits one-way sliding of the radial elements from the first collapsed diameter to the second expanded diameter, but inhibits radial recoil from the second expanded diameter; and a frame element which surrounds at least one radial element in each module.”

By way of yet further illustration, one may use the multi-coated drug-eluting stent described in U.S. Pat. No. 6,702,850, the entire disclosure of which is hereby incorporated by reference in to this specification. This patent describes and claims: “ . . . a stent body comprising a surface; and a coating comprising at least two layers disposed over at least a portion of the stent body, wherein the at least two layers comprise a first layer disposed over the surface of the stent body and a second layer disposed over the first layer, said first layer comprising a polymer film having a biologically active agent dispersed therein, and the second layer comprising an antithrombogenic heparinized polymer comprising a macromolecule, a hydrophobic material, and heparin bound together by covalent bonds, wherein the hydrophobic material has more than one reactive functional group and under 100 mg/ml water solubility after being combined with the macromolecule.”

Referring again to FIG. 35, and to the preferred embodiment depicted therein, the substrate 6004 (such as, e.g., an implantable stent) is disposed within material 6002. The material is preferably biological material, such as the biological material disclosed in published U.S. patent application 2004/0030379. Thus, and as is disclosed in such published patent application, “The present invention provides a method of treatment to reduce or prevent the degree of restenosis or hyperplasia after vascular intervention such as angioplasty, stenting, atherectomy and grafting. All forms of vascular intervention are contemplated by the invention, including, those for treating diseases of the cardiovascular and renal system. Such vascular intervention include, renal angioplasty, percutaneous coronary intervention (PCI), percutaneous transluminal coronary angioplasty (PTCA); carotid percutaneous transluminal angioplasty (PTA); coronary by-pass grafting, angioplasty with stent implantation, peripheral percutaneous transluminal intervention of the iliac, femoral or popliteal arteries, carotid and cranial vessels, surgical intervention using impregnated artificial grafts and the like. Furthermore, the system described in the present invention can be used for treating vessel walls, portal and hepatic veins, esophagus, intestine, ureters, urethra, intracerebrally, lumen, conduits, channels, canals, vessels, cavities, bile ducts, or any other duct or passageway in the human body, either in-born, built in or artificially made. It is understood that the present invention has application for both human and veterinary use.”

Thus, in one embodiment, the material 6002 is biological material such as, e.g., blood, fat cells, muscle, etc.

Referring again to FIG. 35, and to the preferred embodiment depicted therein, a layer of magnetoresistive material 6016 is disposed over the substrate 6004. As is known to those skilled in the art, magnetoresistance is the change in electrical resistance produced in a current-carrying conductor or semi-conductor upon the application of a magnetic field. Reference may be had, e.g., to U.S. Pat. Nos. 6,064,552; 6,178,072; 6,219,205; 6,243,288; 6,256,177; 6,292,336; 6,329,818; 6,340,520 (giant magnetorestive film); U.S. Pat. Nos. 6,387,550; 6,396,734 6,433,792; 6,452,382; 6,483,740; 6,490,140; 6,498,707; 6,501,271 (magnetoresistive effect multilayer sensor); U.S. Pat. Nos. 6,519,119; 6,538,430; 5,538,859; 6,574,061; 6,589,366 (giant magnetoresistance materials based upon Gd—Si—Ge alloys), U.S. Pat. Nos. 6,594,175; 6,612,018; 6,621,667 (giant magnetoresistive sensor), U.S. Pat. Nos. 6,674,664; 6,717,778; 6,730,036 (giant magnetoresistive thin film); and the like. The entire disclosure of each of these United States patents is hereby incorporated by reference into this specification.

Without wishing to be bound to any particular theory, applicants believe that the presence of the magnetoresistive material 6004 helps minimize the presence of eddy currents in substrate 6004 when the assembly 6000 is subjected to a magnetic resonance imaging (MRI) field 6020.

In one preferred embodiment, illustrated in FIG. 35, layers of barrier material 6006 and 6008 are disposed over drug eluting polymer materials 6020 and 6018, respectively. This barrier material is described in U.S. Pat. No. 6,716,444, the entire disclosure of which is hereby incorporated by reference into this specification.

In one preferred embodiment, the diffusivity of the drug through the barrier layer is affected by the application of an external electromagnetic field. The external magnetic field (such as, e.g., field 6020) may be used to heat the nanomagnetic material 6010 and/or the nanomagnetic material 6012 and/or the magnetoresistive material 6016, which in turn will tend to heat the drug eluting polymer 6018 and/or the drug eluting polymer 6020 and/or the barrier layer 6008 and/or the barrier layer 6006. To the extent that such heating increases the diffusion of the drug from the drug-eluting polymer, one may increase the release of such drug from such drug-eluting polymer.

In one embodiment, illustrated in FIG. 35, The heating of the nanomagnetic material 6010 and/or 6012 decreases the effectiveness of the barrier layers 6006 and/or 6008 and, thereby, increases the rate of drug delivery from drug-eluting polymers 6020 and/or 6018.

Referring again to FIG. 35, when an MRI MRI field 6020 is present, the entire assembly 6000, including the biological material 6020, presents a direct current magnetic susceptibility that preferably is plus or minus 1××10⁻³ centimeter-gram-seconds (cgs) and, more preferably, plus or minus 1×10⁻⁴ centimeter-gram-seconds. In one embodiment, the d.c. susceptibility of the stent is equal to plus or minus 1×10⁻⁵ centimeter-gram-seconds. In another embodiment, the d.c. susceptibility of the stent is equal to plus or minus 1×10⁻⁶ centimeter-gram-seconds.

Referring again to FIG. 35, each of the components of assembly 6000 has its own value of magnetic susceptibility. The biological material 6002 has a magnetic susceptibility of S₁. The substrate 6012 has a magnetic susceptibility of S₂ The magnetoresistive 6016 material has a magnetic susceptibility of S₃. The drug-eluting polymeric materials 6018 and 6020 have magnetic susceptibilities of S₉ and S₁₀, respectively.

Each of the components of the assembly 6000 makes a contribution to the total magnetic susceptibility of such assembly, depending upon (a) whether its magnetic susceptibility is positive or negative, (b) the amount of its positive or negative susceptibility value, and (c) the percentage of the total mass that the individual component represents.

In determining the total susceptibility of the assembly 6000, one can first determine the product of Mc and Sc, wherein Mc is the weight fraction of that component (the weight of that component divided by the total weight of all components in the assembly 6000).

In one preferred process, the McSc values for the nanomagnetic material 6016 and the nanomagnetic material 6012 are chosen to, when appropriate, correct for the total McSc values of all of the other components (including the biological material 6002 such that, after such correction(s), the total susceptibility of the assembly 6000 is plus or minus 1××10⁻³ centimeter-gram-seconds (cgs) and, more preferably, plus or minus 1×10⁻⁴ centimeter-gram-seconds. In one embodiment, the d.c. susceptibility of the assembly 6000 is equal to plus or minus 1×10⁻⁵ centimeter-gram-seconds. In another embodiment, the d.c. susceptibility of the assembly 6000 is equal to plus or minus 1×10⁻⁶ centimeter-gram-seconds.

As will be apparent, there may be other materials/components in the assembly 6000 whose values of positive or negative susceptibility, and/or their mass, may be chosen such that the total magnetic susceptibility of the assembly is plus or minus 1××10⁻³ centimeter-gram-seconds (cgs) and, more preferably, plus or minus 1×10⁻⁴ centimeter-gram-seconds. Similarly, the configuration of the substrate may be varied in order to vary its magnetic susceptibility properties and/or other properties. One of these variations is depicted in FIG. 36.

As is known to those skilled in the art, many stents comprise wire. See, e.g., U.S. Pat. No. 6,723,118 (flexible metal wire stent), U.S. Pat. No. 6,719,782 (flat wire stent), U.S. Pat. No. 6,525,574 (wire stent coated with a biocompatible fluoropolymer), U.S. Pat. Nos. 6,579,308, 6,375,660, 6,161,399 (wire reinforced monolayer fabric stent), U.S. Pat. No. 6,071,308 (flexible metal wire stent), U.S. Pat. No. 6,056,187 (modular wire band stent), U.S. Pat. No. 5,999,482 (flat wire stent), U.S. Pat. No. 5,906,639 (high strength and high density intralumina wire stent), and the like. The entire disclosure of each of these United States patents is hereby incorporated by reference into this specification.

FIG. 36 is a sectional view of a wire 6100 which may be used to replace the wire used in conventional metal wire stents. The wire 6100 preferably has a sheath/core arrangement, with sheath 6102 disposed about core 6104.

In one embodiment, the materials chosen for the sheath 6102 and/or the core 6104 afford one both the desired mechanical properties as well as a magnetic susceptibility that, in combination with the other components of the assembly (and of the biological tissue), produce a magnetic susceptibility of plus or minus 1×10⁻³ cgs.

In another embodiment, the materials chosen for the sheath 6102 and/or the core 6104 are preferably magnetoresistive and produce a high resistance when subjected to MRI radiation.

FIG. 37 is a graph 7000 of the relative permeability of a coating 7002 (depicted by triangles in the plot), and a bulk ceramic material 7004 (depicted by squares in the plot), versus the frequency that each of such coatings 7002/7004 interacts with. The term “relative permeability” is well known to those skilled in the art and is discussed, e.g., elsewhere in this specification and in the claims of many United States patents. Reference may be had, e.g., to U.S. Pat. No. 3,966,216 (scanning magnetic head), U.S. Pat. No. 4,236,946 (amorphous magnetic thin films with highly stable easy axis), 4m576,876 (magnetic recording medium), U.S. Pat. No. 4,672,493 (thin film magnetic head), U.S. Pat. No. 4,782,416 (magnetic head having two legs of predetermined saturation magnetization), U.S. Pat. No. 5,105,323 (anistropic magnetic layer), U.S. Pat. No. 5,241,439 (combined read/write thin film magnetic head), U.S. Pat. No. 5,589,842 (microstrip antenna with magnetic substrate), U.S. Pat. No. 5,731,66 (integrated-magnetic filter having a lossy shunt), U.S. Pat. No. 5,858,548 (soft magnetic thin film), U.S. Pat. No. 5,965,214 (methods for coating magnetic tags), U.S. Pat. No. 6,064,546 (magnetic storage apparatus), U.S. Pat. No. 6,084,499 (planar magnetics with segregated flux paths), U.S. Pat. No. 6,225,876 (feed-through EMI filter with a metal flake composite magnetic material), U.S. Pat. No. 6,338,900 (soft magnetic composite material), U.S. Pat. No. 6,371,379 (magnetic tags or makers), U.S. Pat. No. 6,781,492 (superconducting magnetic apparatus), and the like. The entire disclosure of each of these United States patent applications is hereby incorporated by reference into this specification.

The coating 7002 is preferably a coating of the nanomagnetic material described elsewhere in this specification. This material preferably has a magnetization at 2.0 Tesla of from about 0.1 to about 10 electromagnetic units per cubic centimeter. The particle size of the nanomagnetic particles in the coating are preferably from about 3 to about 20 nanometers. Additionally, it is preferred that the concentration of the nanomagnetic particles in the coating be less at the surface of the coating than at its bottom surface, adjacent to the substrate. This is illustrated in FIG. 38.

FIG. 38 is a schematic of a sputtering process 7100 in which a target 7102 is emitting particles 7104 of nanomagnetic material as well as particles 7106 of nonmagnetic material (such as, e.g., aluminum, nitrogen, etc.). The sputtering process 7100 is similar to the sputtering processes discussed elsewhere in this specification.

Referring again to FIG. 38, when the first nanomagnetic particles 7104a approach the substrate 7108, they are attracted by two competing sets of forces. The top surface 7110 of the substrate 7108 provides nucleation centers (not shown) that facilitate the binding of many of the nanomagnetic particles 7104a; and these nucleation centers are sufficient to overcome, at least for these particles 7104a, the attractive forces provided by the magnetic field 7112 of the magnetron 7114.

As the particles 7104a tend to bind to the substrate at the nucleation centers, the new surfaces provided for such binding are not the substrate surface 7110, but the coating of the particles 7104a (and other particles). The coating provides fewer nucleation sites than did the surface 7110; and the more material 7104a (and other material) that is deposited, the weaker the attraction is between the substrate surface 7110 and the nanomagnetic particles 7104a.

Thus, and referring again to FIG. 38, when nanomagnetic particles 7104b are being propelled towards the substrate surface 7110, they are attracted less to such surface 7110 than were the particles 7104a; more of these particles 7104b are attracted back towards the magnetron 7114, and fewer of them are deposited onto the substrate surface 7110.

Similarly, when nanomagnetic particles 7104c are being propelled towards the substrate surface 7110, more of these particles are attracted back towards the magnetron 7114 than were particles 7104b (or 7104a), and fewer of them are deposited onto the substrate surface.

Accordingly, there is a concentration gradient for the nanomagnetic particles 7104. This is best illustrated in FIG. 39, which is a depth profile 8000 of a typical coating 7120 (see FIG. 38), plotting the concentration of the nanomagnetic material 7104 on the surface 7110 (see FIG. 38), and working upwardly from such surface 7110 towards the top surface 8002 of the coating 7120 (see FIG. 38). The depth profile 8000 compares, e.g., the concentration of the magnetic material at the surface 7110 (see point 8004) versus the concentration of the magnetic material at the surface 8002 (see point 8006).

Referring to FIG. 39, it will be seen that the concentration value “A” (which corresponds to the concentration of the magnetic material at or near the surface 7110) is greater than the concentration value “C” (which corresponds to concentration of the magnetic material at or near the top surface 8002 of the coating 7120). The ratio of A/C is preferably at least about 1.5 and, more preferably, is at least about 2.0. As used herein, the term “at or near” refers to the concentration of the material either at the surface in question and/or within the first 0.5 nanometers thereof.

Referring again to FIG. 37, and to the preferred embodiment depicted therein, plots of coated assembly 7020 are presented. Coated assembly 7020 is comprised of a substrate (which preferably is nonmagnetic), nanomagnetic particles, and the coating that such particles comprise.

The plot for coated assembly 7020 shows a relative permeability (plotted on the vertical axis 7010) that increases from a finite value at point 7012 (which corresponds to an a.c. frequency of 0 [or d.c.] at point 7012), up to a maximum relative permeability at point 7014, which corresponds to a critical frequency of the coating 7120; beyond this critical frequency, the ferromagnetic resonance frequency of the coating 7120 will be reached. It will be seen that the ferromagnetic resonance frequency of such coating 7120 on the substrate (which is preferably nonmagnetic) is at least 1 gigahertz (see decreased trend of the curve after point 7014), and more preferably is at least about 5 gigahertz. As is known to those skilled in the art, the precise definition of the ferromagnetic resonance frequency is the frequency at which the real part of the permeability is near 1.

As is known to those skilled in the art, ferromagnetic resonance is the magnetic resonance of a ferromagnetic material. Reference may be had, e.g., to page 7-98 of E. U. Condon et al.'s “Handbook of Physics,” (McGraw-Hill Book Company, New York, N.Y., 1958). Reference also may be had, e.g., to U.S. Pat. Nos. 4,263,374; 4,269,651; 4,853,660; 6,362,533; 6,362,543; 6,501,971; and the like. The entire disclosure of each of these United States patents is hereby incorporated by reference into this specification.

As noted above, the ferromagnetic resonance frequency of the nanomagnetic material is at least 1 gigahertz. By comparison, a bulk ceramic material (such as iron oxide/ferrite material) will have a ferromagnetic resonance frequency that is generally less than about 100 megahertz (see point 7016). The plot 7018 of this ferrite material represents the plot of a material with an average particle size greater than 1 micron. As used in this specification, the term “bulk” refers to a material with an average particle size greater than about 1 micron.

The plot 7018 is a plot of a film comprised of ferrite material that is preferably formed by conventional means, such as plasma spraying. The film has a thickness of about 1 micrometer, as does the nanomagnetic coating 7120.

Thus, the graph 7000 shows the responses of two coatings disposed on substantially identical substrates (which are preferably nonmagnetic) with substantially identical film thicknesses, substantially identical magnetizations at 2.0 Tesla, and substantially identical molar percentages of magnetic material in the films. Both of these samples, at 0 frequency, have the same relative permeability (at point 7012); but their behaviors diverge radically as the alternating current frequency is increased from zero hertz to greater than 1 gigahertz.

Referring to the plot 7020 of the nanomagnetic film, it will be seen that the relative permeability increases at a rate defined by delta permeability/delta frequency; see, e.g., the slope of the triangle 7022, which indicates that the increase in permeability per hertz is from about 1×10⁻¹⁴ to about 1×10⁻⁶, and preferably is from about 1×10⁻¹⁰ to about 1×10⁻⁷. By comparison, and referring to plot 7018 (and to triangle 7024), the permeability of the “bulk” ceramic material decreases at a rate of at least about −1×10⁻⁸.

FIG. 40 is a schematic of a preferred process 9000 in which, when coated stent assembly 9002 is contacted with electromagnetic radiation 9022, images of biological material 9024, 9026, and 9028 are obtained without substantial image artifacts and with good resolution.

The electromagnetic radiation 9022 is preferably radio-frequency alternating current radiation with a frequency of from about 10 to about 300 megahertz. In one preferred embodiment, the frequency is either 64 megahertz, 128 megahertz, or 256 megahertz.

The frequency is preferably in the form of a sine wave with a maximum amplitude 9024 (see FIG. 40). The energy in such electromagnetic radiation 9022 is proportional to the square of the amplitude 9024.

In the preferred embodiment depicted in FIG. 40, the coated stent assembly 9002 is comprised of a stent 9006 on which is disposed a coating 9004. The coating 9004 is similar to the coating 7120 depicted in FIG. 38, and it contains substantially more magnetic particles 9008 (such as, e.g., particles of iron) near the surface 9010 of the stent 9006 than near the top surface 9012 of the coating. There is preferably at least about 1.5 times as many particles of “moiety A” near surface 9010 than near top surface 9012. Without wishing to be bound to any particular theory, applicants believe that this concentration differential along the depth of the coating 9004 facilitates the entry of energy into the interior 9014 of the stent 9006, and it also facilitates the exit of energy from the interior 9014 of the stent 9006 to exterior 9016 of such stent.

Referring again to FIG. 40, and to the preferred embodiment depicted therein, it will be seen that a sensor 9018 is disposed outside of the stent assembly 9002, and that another sensor 9020 is disposed within the interior of the stent 9006. These sensors 9018/9020 are adapted to measure the amount of electromagnetic energy, and the frequency of the electromagnetic energy, that exists at a given spatial point both without and within the stent assembly 9002.

In one preferred embodiment, the stent assembly 9002 has a radio frequency shielding factor of less than about 10 percent and, more preferably, less than about 5 percent. The radio frequency shielding factor is a function of the amount of energy that is blocked from entering the interior 9104 of the stent.

The radio frequency shielding factor can be calculated by first determining the amount of energy in electromagnetic wave 9022. As is known to those skilled in the art, this energy is dependent upon the amplitude 9024 of the energy 9022, being directly dependent upon the square of such amplitude.

After the initial energy of the electromagnetic wave 9022 is determined (and measured by sensor 9018), the amount of such initial energy that passes unimpeded to the interior 9014 of stent assembly 9002 is then determined. Only that energy that has a frequency that is within plus or minus 5 percent of the initial energy of electromagnetic wave 9022 is considered. In one embodiment, only that energy that has a frequency that I within plus or minus two percent of the initial energy of electromagnetic wave 9022 is considered. In an even more preferred embodiment, the frequency of the energy that passes unimpeded into the interior of the stent is within plus or minus one percent of the initial energy.

The “interior energy” is measured by one or more of the sensors 9020; it is also dependent upon the square of the amplitude 9024.

Referring again to FIG. 40, the exterior energy 9030 passes through the stent assembly 9002 (wherein it is identified as energy 9032) until it reaches the interior 9014 of the stent (wherein it is identified as energy 9034). The energy 9034 interacts with biological matter 9024 disposed within the interior of the stent. Depending upon the type and characteristics of the biological matter 9024, a signal 9048 is generated (and measured by sensor 9020); and then this signal passes back through the stent assembly (wherein it is identified as signal 9050) and to the outside of the stent assembly (wherein it is identified as signal 9052).

Without wishing to be bound to any particular theory, applicants believe that the presence of the concentration gradient in coating 9004 of the moiety A (discussed elsewhere in this specification) facilitates the substantially unimpeded exit of signal 9048 through the stent assembly 9002 (wherein it is identified as signal 9050) and to the exterior of the stent assembly (wherein it is identified as signal 9052). The term “substantially unimpeded) refers to the fact that the signal 9052 contains at least 90 percent (and preferably at least 95 percent) of the energy of signal 9048 and has a frequency which is within plus or minus 5 percent (and preferably plus or minus 2 percent) of the frequency of signal 9048.

Referring again to FIG. 40, the exterior energy 9036 passes through the stent assembly 9002 (wherein it is identified as energy 9038) until it reaches the interior 9014 of the stent (wherein it is identified as energy 9040). The exterior energy 9036 and the interior energy 9040 are preferably substantially identical to the exterior energy 9030 and the interior energy 9034, and also to the exterior energy 9042 and to the interior energy 9046.

Referring again to FIG. 40, the energy 9040 interacts with biological matter 9026 disposed within the interior of the stent. Depending upon the type and characteristics of the biological matter 9026, a signal 9054 is generated (and measured by sensor 9020). This signal 9054 will differ from signal 9048 (and also from signal 9056) in that biological matter 9026 differs from biological matter 9024 and biological matter 9028 in either its size, composition, shape, etc.

Referring again to FIG. 40, the signal 9054 passes back through the stent assembly (wherein it is identified as signal 9058) and to the outside of the stent assembly (wherein it is identified as signal 9062).

Without wishing to be bound to any particular theory, applicants believe that the presence of the concentration gradient in coating 9004 of the moiety A (discussed elsewhere in this specification) facilitates the substantially unimpeded exit of signal 9054 through the stent assembly 9002 (wherein it is identified as signal 9058) and to the exterior of the stent assembly (wherein it is identified as signal 9062). The term “substantially unimpeded) refers to the fact that the signal 9062 contains at least 90 percent (and preferably at least 95 percent) of the energy of signal 9040 and has a frequency which is within plus or minus 5 percent (and preferably plus or minus 2 percent) of the frequency of signal 9040.

Referring again to FIG. 40, the exterior energy 9042 passes through the stent assembly 9002 (wherein it is identified as energy 9044) until it reaches the interior 9014 of the stent (wherein it is identified as energy 9046). The exterior energy 9042 and the interior energy 9046 are preferably substantially identical to the exterior energy 9030 and the interior energy 9036.

Referring again to FIG. 40, the energy 9046 interacts with biological matter 9028 disposed within the interior of the stent. Depending upon the type and characteristics of the biological matter 9028, a signal 9056 is generated (and measured by sensor 9020). This signal 9056 will differ from signal 9048 (and also from signal 9054) in that biological matter 9028 differs from biological matter 9024 and biological matter 9026 in either its size, composition, shape, etc.

Referring again to FIG. 40, the signal 9056 passes back through the stent assembly (wherein it is identified as signal 9060) and to the outside of the stent assembly (wherein it is identified as signal 9064).

Without wishing to be bound to any particular theory, applicants believe that the presence of the concentration gradient in coating 9004 of the moiety A (discussed elsewhere in this specification) facilitates the substantially unimpeded exit of signal 9056 through the stent assembly 9002 (wherein it is identified as signal 9060) and to the exterior of the stent assembly (wherein it is identified as signal 9064). The term “substantially unimpeded) refers to the fact that the signal 9064 contains at least 90 percent (and preferably at least 95 percent) of the energy of signal 9056 and has a frequency which is within plus or minus 5 percent (and preferably plus or minus 2 percent) of the frequency of signal 9056.

The “exterior energies” 9030, 9036, and 9042 will all be substantially identical to each other, as will their corresponding “intermediate energies” 9032/9038/9044 and “interior energies” 9034/9040/9046. However, because each of biological materials 9024, 9026, and 9028 differs from the others, the interaction of these biological matters with interior energies 9034/9040/9046 will produce differing interior signals 9048/9054/9056, differing intermediate signals 9050/9058/9060, and differing exterior signals 9052/9062/9064.

However, although the process 9000 produces differing interior signals 9048/9054/9056, differing intermediate signals 9050/9058/9060, and differing exterior signals 9052/9062/9064, it produces a substantially uniform response along the length of the stent assembly 9002. The ratio of the energy of signal 9052 to signal 9048 (their frequencies being within plus or minus 5 percent of each other), and the ratio of the energy of signal 9062 to signal 9058 (their frequencies being within plus or minus 5 percent of each other), and the ratio of the energy of signal 9064 to signal 9056 (their frequencies being within plus or minus 5 percent of each other), will each be substantially identical to each other, and all of them will be within the range of from 0.9 to 1.0, as described above.

Without wishing to be bound to any particular theory, applicants believe that this uniformity of imaging response is due to the substantially uniform nature of the coating 9004 disposed on the stent 9006. Because the concentration differential of the moiety A is substantially identical along the length of the stent 9006, the imaging response of the stent is also substantially identical along its entire length. This is schematically illustrated by graph 9027.

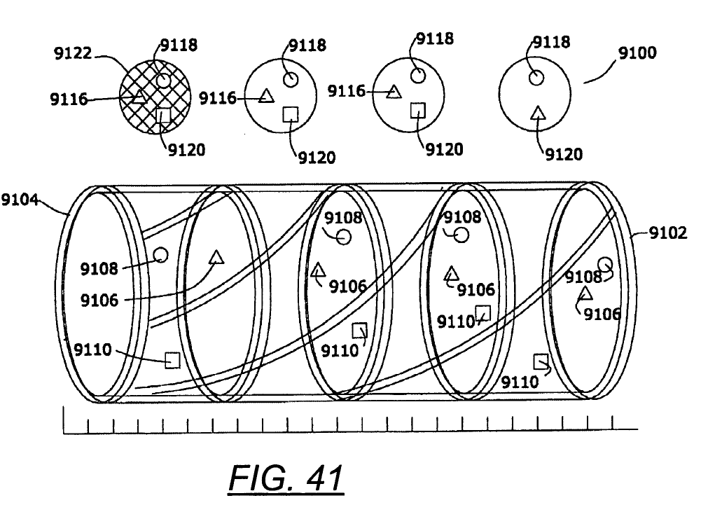

FIG. 41 is a schematic of a coated stent 9102 on which is disposed a nanomagnetic coating 9104 and within which is disposed biological materials 9106, 9108, and 9110. In the embodiment depicted, the images produced of these materials when they are subjected to MRI imaging with a 64 megahertz radio frequency source and 1.5 Tesla d.c. field are shown as 9116, 9118, and 9120. Similar images will be produced with 128 megahertz and 256 megahertz radio frequency fields.

When the coating 9104 is not disposed on the stent 9102, a “smeared” set of images 9122 is produced that makes it difficult for, e.g., a physician to clearly distinguish the images 9116, 9118, and 9120. When, however, the coating 9104 is disposed on the stent 9102, the images 9116, 9918, and 9120 are presented with good resolution.

As is known to those skilled in the art, resolution is the ability of a system to reproduce the points, lines, and surfaces in an object as separate entities in the image. A substantial amount of patent literature has been devoted to the resolution of, e.g., MRI images. Reference may be had, e.g., U.S. Pat. No. 4,684,891 (rapid magnetic resonance imaging using multiple phase encoded spin echoes in each of plural measurement cycles), U.S. Pat. No. 4,857,846 (rapid MRI using multiple receivers), U.S. Pat. No. 4,881,034 (switchable MRI RF coil arrangement), U.S. Pat. No. 4,888,552 (magnetic resonance imaging), U.S. Pat. No. 4,954,779 (correction for eddy current caused phase degradation), U.S. Pat. No. 5,361,764 (magnetic resonance imaging foot coil assembly), U.S. Pat. No. 5,399,969 (analyzer of gradient power usage for oblique MRI imaging), U.S. Pat. No. 5,438,263 (method of selectable resolution magnetic resonance imaging), U.S. Pat. No. 5,646,529 (system for producing high-resolution magnetic resonance images), U.S. Pat. No. 5,818,229 (correction of MR imaging pulse sequence), U.S. Pat. No. 6,317,620 (method and apparatus for rapid assessment of stenosis severity), U.S. Pat. No. 6,425,864 (method and apparatus for optimal imaging of the peripheral vasculature), U.S. Pat. No. 6,463,316 (delay based active noise cancellation for magnetic resonance imaging), U.S. Pat. No. 6,556,845 (dual resolution acquisition of magnetic resonance angiography data), U.S. Pat. No. 6,597,173 (method and apparatus for reconstructing zoom MR images), U.S. Pat. No. 6,603,992 (method and system for synchronizing magnetic resonance image acquisition to the arrival of a signal-enhancing contrast agent), U.S. Pat. No. 6,720,766 (thin film phantoms and phantom systems), U.S. Pat. No. 6,741,880 (method and apparatus for efficient stenosis identification and assessment using MR imaging), and the like. The entire disclosure of each of these United States patent is hereby incorporated by reference into this specification.

Referring again to FIG. 41, and in the preferred embodiment depicted, the objects 9106, 9108, and 9110 preferably have maximum dimensions of about 1 millimeter. These objects are accurately imaged with the coated stent of this invention; thus, such coated stent is said to have a resolution of at least about 1 millimeter. In one embodiment, the resolution is at least about 0.5 millimeters.

The process and apparatus of this invention allows one to avoid the well known Faraday cage effects that limit the visibility of images of objects within a stent. If the stent 9102 did not have the coating 9104, it is likely that, at best, a smeared image would be produced because of the Faraday cage effects. Such a smeared image is indicated as 9122, and it is substantially useless in helping one to accurately determine what objects are disposed within the stent.

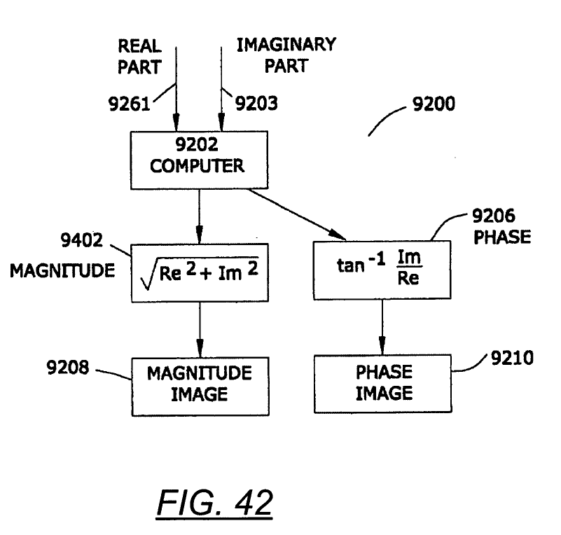

In one preferred embodiment, phase imaging is used with the coated stent 9100. The phase imaging process 9200 is schematically illustrated in FIG. 42.

The phase imaging process is well known to those skilled in the art and widely described in the patent literature. Reference may be had, e.g., to U.S. Pat. No. 4,878,116 (vector lock-in imaging system), U.S. Pat. No. 5,335,602 (apparatus for all-optical self-aligning holographic phase modulation and motion sensing), U.S. Pat. No. 5,447,159 (optical imaging for specimens having dispersive properties), U.S. Pat. No. 5,633,714 (preprocessing of image amplitude and phase data for CD and OL measurement), U.S. Pat. No. 5,760,902 (method and apparatus for producing an intensity contrast image from phase detail in transparent phase objects), U.S. Pat. No. 5,995,223 (apparatus for rapid phase imaging interferometry), U.S. Pat. No. 6,809,845 (phase imaging using multi-wavelength digital holography), U.S. Pat. No. 6,853,191 (method of removing dynamic nonlinear phase errors from MRI data), and the like. The entire disclosure of each of these United States patents is hereby incorporated by reference into this specification.

Referring again to FIG. 42, in step 9202 the real part 9201 and the imaginary part 9203 are processed in computer 9202. These parts are discussed in FIG. 13-18 of Ray H. Hashemi's “MRI The Basics,” (Lippincott Williams & Wilkins, Philadelphia, Pa., 2004) at page 158, wherein it is disclosed that “The FTs of the real and imaginary k-spaces provide the real and imaginary images, respectively.” At pages 156-157 of the Hashemi et al. text, it is disclosed that “We discussed two components of the data space, namely, the real and imaginary components. Their respective Fourier transforms provide the real and imaginary components of the image (FIG. 13-18).”

The Hashemi et al. text also discloses that (at page 157) “Recall that a given complex number c=a+ib, with a being the real and b the imaginary component . . . . This concept can be applied to the real and imaginary components of the image (FIG. 13-18) to generate the magnitude and the phase images. The magnitude image (modulus) is what we deal with most of the time in MR imaging. The phase image is used in cases in which the direction is important. An example is phase contrast MR angiography . . . .”\

Referring again to FIG. 42, and in step 9204 thereof, the magnitude image 9208 is derived by calculating the square root of the [(real image)²+(imaginary image)²]. By comparison, the phase image 9210 is derived by calculating the arc tangent of the [imaginary image/real image].

Without wishing to be bound to any particular theory, applicants' believe that their nanomagnetic coating is ideally suited for phase imaging. Some of the reasons for this suitability are illustrated in FIG. 43.

Referring to FIG. 43, plot 9300 represents the energy input to the device to be imaged; this energy is often 64 megahertz radio frequency energy.

Plot 9302 is the output signal generated from a stent with biological matter disposed therein, wherein the stent is not coated with the nanomagnetic material of this invention. As will be apparent, this output signal has a loss of coherence (see points 9304 and 9306) due to the Faraday cage effect.

Plot 9308 shows the image from a coated stent with biological matter disposed therein, wherein the coating is the nanomagnetic material of this invention . . . the bottom shows the signal out with nanomagnetic coating. This is a coherent image (compare image 9302) whose phase is shifted by less than about 90 degrees and, more preferably, less than about 45 degrees. In one preferred embodiment, depicted in FIG. 43, the phase angle 9310 is preferably less than about 30 degrees.

Referring again to FIG. 43, the coherent signal 9308 is preferably substantially identical to the input signal, except for its phase shift 9310. It has substantially the same amplitude, substantially the same frequency, and substantially the same shape.

In one embodiment of the process of this invention, using the phase shift 9310, one can reconstruct the image of the actual object inside the stent by reference to the stent and with the use of phase imaging.

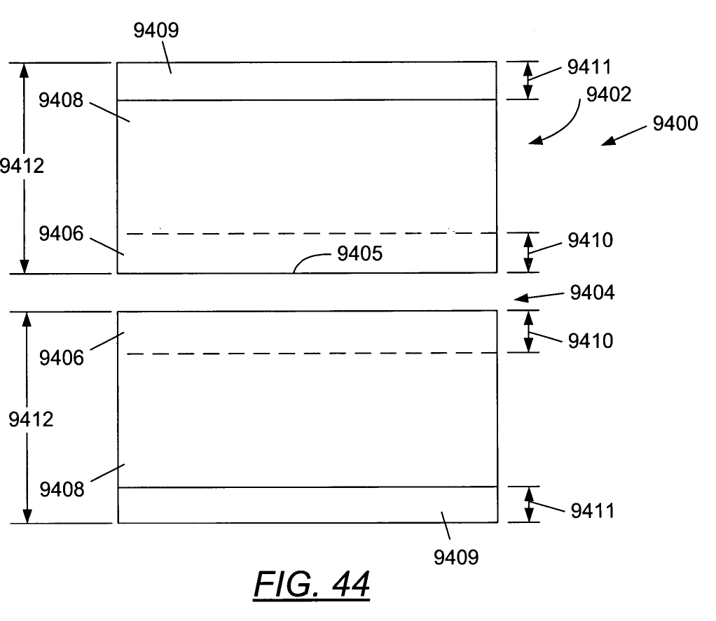

FIG. 44 is a schematic of a coated stent assembly 9400 comprised of a coating 9402 disposed circumferentially around a stent 9404. Without wishing to be bound to any particular theory, applicants believe that, in order to “choke” any particular section of the stent 9404 (such as, e.g., section 9405), the coating 9402 should preferably be circumferentially disposed around the entire periphery of such section of the stent. Applicants also believe that such circumferential coating effectively blocks the flow of induced eddy currents or loop currents through the section of sections in question.

Referring again to FIG. 44, and in the preferred embodiment depicted therein, it will be seen that coating 9402 is comprised of a first section 9406, a second section 9408, and a third section 9409. Each of these sections has different physical properties.

The first section 9406 has a thickness 9410 that preferably is from about 50 to about 150 nanometers. In one preferred embodiment, the thickness 9410 is from about 5 to about 15 percent of the total thickness 9412 of the coating, which often is in the range of from about 400 to about 1500 nanometers.

The third (top) section 9409 preferably has a thickness 9411 that is at least 10 nanometers and, more preferably, from about 10 to about 100 nanometers. In one embodiment, the thickness 9411 is from about 0.5 to about 15 percent of the total thickness 9412.

Magnetic material, such as the “moiety A” described elsewhere in this specification, is disposed throughout the entire thickness 9412 of the coating 9402, but more of it is disposed on a fractional mole per unit volume basis in the first coating than in the third coating. The first section 9406 preferably has at least 1.5 times as greater the number of fractional moles of moiety A per cubic centimeter than does the middle section 9408; and the first section 9406 preferably has at least 2.0 times as great the number of fractional moles of moiety A than does the top section 9409.

The relative permeability of the first section 9406 is preferably greater than about 2. The relatively permeability of the third section 9409 preferably is less than about 2 and, more preferably, less than about 1.5.

The resistivity of the third section 9409 is at least 10 times as great as the combined average resistivity of sections 9406 and 9408. In one embodiment, the resistivity of section 9409 is at least 100 times as great as the combined average resistivity of sections 9406 and 9408. In one embodiment, the combined average resistivity of sections 9406 and 9408 is from about 10⁸ to about 10⁻³. In another embodiment, the resistivity of section 9409 is from about 10¹⁰ to about 10³ and, more preferably, from about 10⁹ to about 10⁷.

In one embodiment, the section 9408 has a relative dielectric constant that is at least 1.2 times as great as the relative dielectric constant from section 9406, and is also at least 1.2 times as great as the relative dielectric constant 9409.

FIG. 45 is a sectional view of one preferred coated ring assembly 9500 comprised of a conductive ring 9502 and a layer of nanomagnetic material 9504 disposed around such conductive ring 9502, including its top and bottom surfaces. The conductive ring 9502 preferably comprises a section of a stent.

The conductive ring 9502 may be comprised of conductive material, such as copper, stainless steel, Nitinol, and the like. In one preferred embodiment, the conductive ring is Nitinol.

As is known to those skilled in the art, Nitinol is a paramagnetic intermetallic compound of nickel and titanium. Reference may be had, e.g., to U.S. Pat. No. 5,147,370 (Nitinol stent for hollow body conduits), U.S. Pat. No. 5,290,289 (Nitinol spinal instrumentation and method for surgically treating scoliosis), U.S. Pat. No. 5,681,344 (esophopgeal dilation balloon catheter containing flexible Nitinol wire), U.S. Pat. No. 5,916,178 (steerable high support guidewire with thin wall Nitinol tube), U.S. Pat. No. 6,706,053 (Nitinol alloy design for sheath deployable and resheathable vascular devices), U.S. Pat. No. 6,855,161 (radiopaque nitinol alloys for medical devices), and the like. The entire description of each of these United States patents is hereby incorporated by reference into this specification.

Referring again to FIG. 45, and in the preferred embodiment depicted therein, the wire on the ring 9502 preferably has a diameter of from about 0.8 to about 1.2 millimeters. The ring 9502 preferably has a inner diameter of from about 4 to about 7 millimeters and, more preferably, from about 5 to about 6 millimeters.

When the coated ring assembly 9500 is subjected to an MRI field (that is, e.g., comprised of a radio frequency wave of 64 megahertz), the strongest applied radio frequency field is in the middle 9506 of the ring. It in order to maximize the likelihood of imaging biological material (not shown) disposed within the interior 9508 of the ring 9502, I is preferred that the ring 9502 be coated around its entire periphery with the nanomagnetic material 9504 that contains a higher concentration of magnetic material near the surface of the ring than away from the surface of the ring (see FIG. 40 and the discussion of coating 9002). Such a coating of this type of nanomagnetic material will produce the desired “choking effects” and will thus enhance the imageability of the material disposed within the interior 9508 of the stent.

For optimum imageability under MRI imaging conditions, it is preferred that coated assembly have an inductance within the range of from about 0.1 to about 5.0 nanohenries, and that it also have a capacitance of from about 0.1 to about 10 nanofarads. Referring again to FIG. 45, a material with a high dielectric constant (such as aluminum nitride) is used to provide a coating 9510.

The coating 9510 preferably should contain material with a dielectric constant of from about 4 to about 700 and, more preferably, from about 8 to about 100. Suitable materials include, e.g., aluminum nitride, barium titanate, bismuth titanate, etc.

The material chosen for the coating 9510, and the materials chosen for the coatings 9504, should preferably have a resistance such that the bandwidth of the filter formed by these components is from about 1 to about 5 percent of the frequency of MRI radiation.

In one preferred embodiment, the coatings 9504/9510 comprise a bandpass filter. As is known to those skilled in the art, a bandpass filter is a filter designed to transmit a band of frequencies with negligible loss while rejecting all other frequencies. In the case of 64 megahertz MRI radiation, the bandwidth of such filter is preferably from about 0.5 to about 4.0 megahertz.

FIG. 46 illustrates a coated stent assembly 9501 that is similar in many respects to the coated stent assembly 9500 (see FIG. 45) but differs therefrom in that a thin layer 9505 of FeAl with a thickness of from about 1 to about 20 nanometers (and preferably of from about 8 to about 12 nanometers) is disposed between the layers 9504 of nanomagnetic material and the layers 9510 of dielectric material. Without wishing to be bound to any particular theory, applicants believe that the layer of FeAl disposed over the nanomagnetic material 9504 provides additional magnetic properties (because its concentration of the A moiety is often higher than the concentration of the A moiety in the nanomagnetic material 9504) and it also increases the “choking effect” (because of the increased concentration of the A moiety) and the inductance value.

In this embodiment, it is still preferred to have the inductance within the range of from about 0.1 to about 5.0 nanohenries, and the capacitance of be from about 0.1 to about 10 nanofarads. The addition of the FeAl layer(s) 9505 often helps to “tune” the assembly to obtain the optimal inductance and capacitance values with the aforementioned ranges.

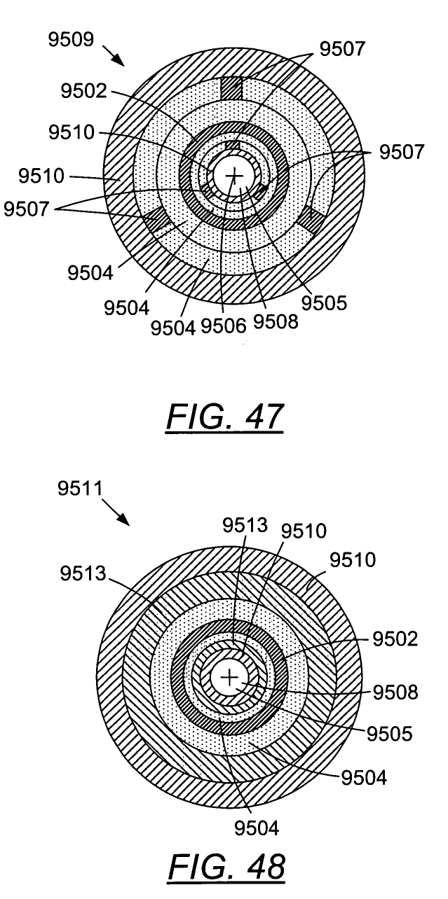

FIG. 47 is a sectional view of a coated stent assembly 9509 that is comprised of conductive vias.9507. As will be apparent, this FIG. 47, and the other Figures, are purposely not drawn to scale in order to facilitate the depiction of certain important details such as, e.g., vias 9507.

One may create vias, such as, e.g., via 9507. by conventional means. Thus, e.g., one may create vias by the means disclosed in U.S. Pat. No. 3,988,823, the entire disclosure of which is hereby incorporated by reference into this specification. This patent claims “1 . . . A method for fabricating a multilevel interconnected large scale integrated microelectronic circuit including vias therein having 0.5 mil and smaller openings for interlayer electrical communication of active devices and unit circuits on a silicon wafer in the microelectronic circuit, comprising the steps of: preparing a silicon wafer with active devices therein and interconnecting the active devices into functional unit circuits at a first level of aluminum metallization including means defining signal-connect pads terminating the unit circuits, by metal evaporation, masking and etching techniques; depositing a layer of pyrolytic silicon dioxide of approximate 0.5 micron thickness on the first level of metallization within a pyrolytic silicon dioxide deposition chamber for passivating the first level and for creating undesired openings in the pyrolytic layer; depositing a layer of photoresist material on the layer of pyrolytic silicon dioxide; placing on the photoresist layer a first mask defining positions of via openings to be etched in the layer of pyrolytic silicon dioxide and to be positioned over the signal-connect means; exposing the photoresist layer through the mask and thereafter removing the mask; developing, baking and further processing the exposed photoresist layer for forming therefrom an etch-resistant mask on the pyrolytic silicon dioxide layer with means defining openings in the etch-resistant mask positioned above the positions of the vias to be formed in the pyrolytic silicon dioxide layer; etching the pyrolytic silicon dioxide layer through the opening means in the etch-resistant mask by applying a mixture of acetic acid, ammonium fluoride and hydrogen fluoride over the etch-resistant mask for forming the vias having at most 0.5 mil openings; stripping the etch-resistant mask from and thereafter cleaning the etched pyrolytic silicon dioxide layer; forming aluminum-magnesium masks defining mushroom configurations, each comprising an aluminum crown and a magnesium stem on the etched pyrolytic silicon dioxide layer, with the stems covering the vias in the etched pyrolytic silicon dioxide layer; sputter depositing a layer of silicon dioxide of a thickness sufficient for adequate insulation over the pyrolytic silicon dioxide layer and over the mushroom-masks in a radio-frequency system for providing tapered deposits at the base of the stems and for closing any of the undesired openings in the pyrolytic silicon dioxide layer; removing the mushroom-masks by immersing the wafer in a dilute nitric acid bath for dissolving the magnesium stems of the mushroom-masks and thereby for floating-out the mushroom-masks for forming means in the RF-sputtered silicon dioxide layer defining openings of at least 3 mil diameters over the vias having at most the 0.5 mil openings in the pyrolytic silicon dioxide layer; forming a second level of aluminum metallization defining interconnections among the active devices and the unit circuits over the RF-sputtered silicon dioxide layer and the pyrolytic silicon dioxide layer exposed and surrounded by the opening means for making low resistance electrical contact through the vias and for effecting continuity of the second level of aluminum through the opening means and the vias; further processing of the silicon wafer from the second level of metallization into the integrated microelectronic circuit; and annealing of the circuit at approximately 400° C. for approximately 16 hours for reducing any contact resistance through the opening means and the vias to a uniform, acceptable level.”

By way of further illustration, and referring to U.S. Pat. No. 4,753,709, the entire disclosure of which is hereby incorporated by reference into this disclosure, one may form vias by the etching process of claim 1 of this patent, which describes “1. A method for fabricating an integrated circuit on a semiconductor chip, comprising: forming a conductive interconnection layer comprised of silicon; forming a silicide film on the surface of said conductive layer; depositing a dielectric film covering said conductive layer; etching said dielectric film so that selected locations of said silicide film on said conductive layer are exposed; and depositing a metal interconnection layer.”

By way of yet further illustration, and referring to U.S. Pat. No. 6,784,096, the entire disclosure of which is hereby incorporated by reference into this specification, one may form barrier layers in high aspect vias by a process comprising the steps of “A method of forming a barrier layer comprising: (a) providing a substrate having a metal feature; a dielectric layer formed over the metal feature; and a via having sidewalls and a bottom, the via extending through the dielectric layer to expose the metal feature; (b) forming a barrier layer over the sidewalls and bottom of the via using atomic layer deposition, the barrier layer having sufficient thickness to servo as a diffusion barrier to at least one of atoms of the metal feature and atoms of a used layer formed over the barrier layer; (c) removing at least a portion of the barrier layer from the bottom of the via by sputter etching the substrate within a high density plasma physical vapor deposition (HDPPVD) chamber having a plasma ion density of at least 1010 ions/cm3 and configured for seed layer deposition, wherein a bias is applied to the substrate during at least a portion of the sputter etching; and (d) depositing a seed layer on the sidewalls and bottom of the via within the HDPPVD chamber.”

The aforementioned patents are merely illustrative of many United States patents that describe via forming processes. Thus, e.g., by way of yet further illustration, one may use the via forming processes described in U.S. Pat. No. 4,258,468 (forming vias through multilayer circuit boards), U.S. Pat. No. 4,670,091 (forming vias on integrated circuits), U.S. Pat. No. 4,780,770 (planarized process for forming vias), U.S. Pat. No. 5,091,339 (trenching techniques for forming vias and channels), U.S. Pat. No. 5,108,562 (electrolytic method for forming vias), U.S. Pat. No. 5,293,025 (method for forming vias in multilayer circuits), U.S. Pat. No. 5,424,245 (forming vias through two-sided substrate), U.S. Pat. No. 5,510,294 (forming vias for multilevel metallization), U.S. Pat. No. 5,593,606 (ultraviolet laser system and method for forming vias in multi-layered targets), U.S. Pat. No. 5,593,921 (method of forming vias), U.S. Pat. No. 5,683,758 (method of forming vias), U.S. Pat. Nos. 5,825,076, 5,861,673 (method for forming vias in multi-level integrated circuits), U.S. Pat. No. 5,874,369 (method for forming vias in a dielectric film), U.S. Pat. No. 5,904,566 (reactive ion etch method for forming vias), U.S. Pat. No. 6,037,262 (process for forming vias and trenches for metal lines in multiple dielectric layers), U.S. Pat. No. 6,096,655 (method for forming vias in an insulation layer for a dual-damascene multilevel interconnection structure), U.S. Pat. No. 6,140,221 (method for forming vias through porous dielectric materials), U.S. Pat. No. 6,180,518 (method of forming vias in a low dielectric constant material), U.S. Pat. No. 6,429,049 (laser method for forming vias), U.S. Pat. No. 6,433,301 (beam shaping and projection imaging with solid state UV Gaussian beam to form vias), U.S. Pat. No. 6,475,889 (method of forming vias in silicon carbide), U.S. Pat. No. 6,518,171 (dual damascene process), U.S. Pat. Nos. 6,649,497, 6,791,060, and the like. The entire disclosure of each of these United States patents is hereby incorporated by reference into this specification.

Referring again to FIG. 47, and to the preferred embodiment depicted therein, the filled vias 9507 preferably extend between nanomagnetic material 9504 and dielectric material 9510. These filled vias which, in one embodiment are filled with aluminum, provide yet another means to “tune” the coated assembly 9509 so that it preferably has an inductance within the range of from about 0.1 to about 5.0 nanohenries, and a capacitance of from about 0.1 to about 10 nanofarads. Without wishing to be bound to any particular theory, applicants believe that capacitance is formed between two adjacent dielectric materials separated by a conductor. Thus, constructs 9510/9507/9510 form capacitance, as do constructs 9510/9504/9510.

is a sectional view of a coated stent assembly 9511 in which a layer 9513 of conductive material is preferably disposed between a layer 9504 of nanomagnetic material and a layer 9510 of dielectric material. The use of the conductive material (such as aluminum) disposed between layers of “dielectric material” provides some capacitance. Thus e.g., a construct of FeAlN/Al/FeAlN provides some capacitance, inasmuch as the material FeAlN/Al/AlN provides some capacitance to which the FelAlN and the AlN layers contribute. In this construct, it is preferred to keep the conductive layer 9513 (such as the aluminum layer 9513) relatively thin, preferably less than about 100 nanometers.

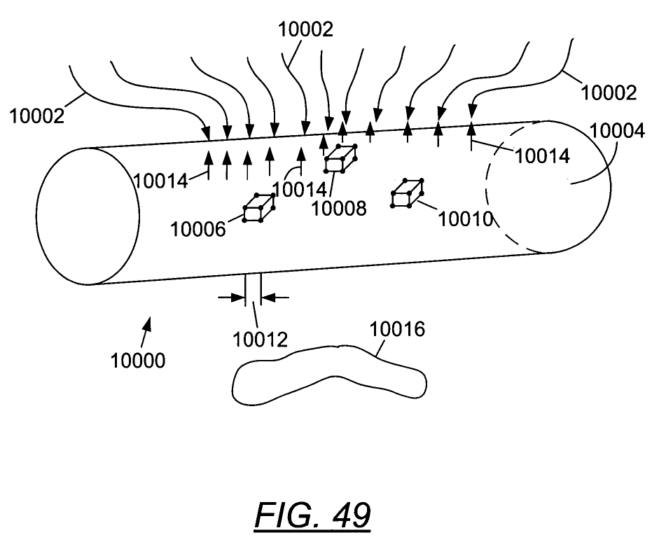

FIG. 49 is a schematic illustration of the behavior of a prior art stent 10000 that is being subjected to MRI radiation (not shown) comprised of lines of flux 10002; in the embodiment depicted, the MRI radiation is 64 megahertz radio frequency electromagnetic radiation, but it could be, e.g., 32-megahertz, 128 megahertz, or 256 megahertz radio frequency electromagnetic radiation.

Referring again to FIG. 49, the prior art stent 10000 may, e.g., be similar to the stent described and claimed in U.S. Pat. No. 6,280,385, the entire disclosure of which is hereby incorporated by reference into this specification. Claim 10 of this patent describes “10. A stent imageable by a magnetic resonance imaging system and having a skeleton which can be unfolded, the stent comprising at least one passive resonance circuit having an inductor and a capacitor forming a closed-loop coil arrangement and whose resonance frequency corresponds to a resonance frequency of high-frequency radiation applied by the magnetic resonance imaging system.

The mechanism by which the stent of U.S. Pat. No. 6,280,385 functions is disclosed in column 3 of the patent, wherein it is disclosed that “These and other objects are achieved by the present invention, which comprises a stent which is to be introduced into the examination object. The stent is provided with an integrated resonance circuit which induces a changed response signal in a locally defined area in or around the stent that is imaged by spatial resolution. The resonance frequency is essentially equal to the resonance frequency of the applied high-frequency radiation of the magnetic resonance imaging system. Since that area is immediately adjacent to the stent (either inside or outside thereof), the position of the stent is clearly recognizable in the correspondingly enhanced area in the magnetic resonance image. Because a changed signal response of the examined object is induced by itself, only those artifacts can appear that are produced by the material of the stent itself.”

U.S. Pat. No. 6,280,385 also discloses that “Due to a clear imaging of the stent in the magnetic resonance image, a precise position determination is possible. Furthermore, based on the changed signal conditions, improved flow measurement of the medium flowing through the stent or along the stent is now possible. Use is made of the fact that different excitation is present inside and outside the stent.”

U.S. Pat. No. 6,280,385 also discloses that “The present invention is based on the surprising discovery that suitable resonance circuits can be provided or disposed on a stent itself. Advantageously, the present invention preferably provides that the inductor and capacitor defining the resonance circuit are formed by the material of the stent, thereby resulting in an additional synergistic effect. It is also within the framework of this invention to form the inductor and capacitor as separate components on the stent.”

U.S. Pat. No. 6,280,385 also discloses that “According to the invention, the signal response of the spins within the inductance is changed. Two processes contribute to this. On the one hand, the resonance circuit tuned to the resonance frequency is excited by the application of high-frequency radiation and the nuclear spins detected by the field of the resonance circuit experience amplified excitation through the local amplification of the alternating magnetic field in or near the inductance. In other words, protons detected by the field lines of the induced magnetic field are deflected at a larger angle than the protons on the outside of this induced magnetic field. An increased flip of the nuclear spins results. Accordingly, the signal response sensed by a receptor coil and evaluated for imaging can be amplified. It is furthermore possible that only the spins within the inductance experience saturation and that the signal is diminished with regard to the environment. In both cases, a change in signal response is apparent.”

U.S. Pat. No. 6,280,385 also discloses that “On the other hand—independent of amplified excitation—the magnetic resonance response signals of the protons within the inductance are amplified. The inductance thus bundles the magnetic field lines originating from the spins within the inductance, which results in an amplified signal emission and an application to a corresponding receptor coil that receives the amplified signals and transmits them for magnetic resonance imaging. This effect is described in the publication by J. Tanttu: “Floating Surface Coils”, in: XIV ICMBE and VII ICMP, Espoo, Finland 1985”.”

According to U.S. Pat. No. 6,280,385, priority for this patent was based upon German patent application 197 46 735, filed on Oct. 13, 1997 (see the front page of the United States patent). This German patent application was also referred to in column 2 of U.S. Pat. No. 6,767,360 of Alt, the entire disclosure of which is hereby incorporated by reference into this specification. In this Alt patent, in the paragraph beginning at line 50 of column 2, it is disclosed that “In German application 197 46 735.0, which was filed as international patent application PCT/DE98/03045, published Apr. 22, 1999 as WO 99/19738, Melzer et al (Melzer, or the 99/19738 publication) disclose an MRI process for representing and determining the position of a stent, in which the stent has at least one passive oscillating circuit with an inductor and a capacitor. According to Melzer, the resonance frequency of this circuit substantially corresponds to the resonance frequency of the injected high-frequency radiation from the magnetic resonance system, so that in a locally limited area situated inside or around the stent, a modified signal answer is generated which is represented with spatial resolution. However, the Melzer solution lacks a suitable integration of an LC circuit within the stent.”

Without wishing to be bound to any particular theory, applicants believe that FIG. 49 represents what occurs with the stent disclosed in U.S. Pat. No. 6,280,385 when it is exposed to the aforementioned MRI radiation; and FIG. 49 illustrates how the “integrated resonance circuit” of the stent of such patent influences imaging of objects disposed within the lumen of such stent.

Referring to FIG. 49, and in the embodiment depicted, disposed within the lumen 10004 of the stent 10000 are objects 10006, 10008, and 10010. In the embodiment depicted, the objects 1006/10008/10010 are square shaped with a width/length 10012 of about 1 millimeter.

Referring again to FIG. 49, and to the embodiment depicted therein, it is believed that the “passive resonant circuit” (not shown) of the stent 10000 concentrates and distorts the flux lines 10002 produced by the MRI radio frequency electromagnetic field. Without wishing to be bound to any particular theory, it is believed that such distortion occurs because the resonance effect produced by stent assembly 10000 occurs over the whole stent (and also beyond the stent in the surrounding area) thereby capturing magnetic flux lines 10002 from nearby areas and concentrating them in the area of stent 10000. Consequently, the magnetic field that previously had been homogeneous prior to the time it came near the stent 100000 (not shown) now becomes distorted and non uniform.

At least some of these flux lines 10002 interact with objects 10006 and/or 10008 and/or 10010 and cause the generation of return signals 10014. These signals 10014 are then processed by the MRI machine (not shown) and converted into an image 10016.

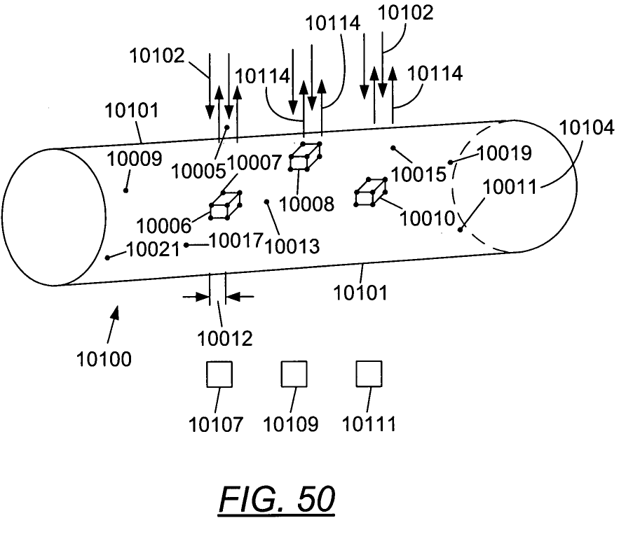

As will be apparent, the image 10016 is somewhat “smeared,” i.e., it does not allow one to distinguish the existence of and/or the separate identity of and/or the size of objects 10006, 10008, and/or 10010. Without wishing to be bound to any particular theory, applicants believe that this “smearing” occurs because the resonance effects in the stent assembly 10000 are not localized and that the concentration of flux lines at different points within the lumen 10004 is not substantially uniform. By comparison, in the stent 10100 depicted in FIG. 50, the resonance effects produced are “localized” over relatively small areas, the magnetic flux lines 10102 are not distorted, and the concentration of the magnetic flux lines is substantially uniform within the entire area of the lumen 10104.

As is known to those skilled in the art, one may measure the concentration of alternating current magnetic flux lines at a particular point in space with, e.g., a Hall probe or a gaussmeter

Hall probes, and their use in measuring magnetic fields, are well known to those skilled in the art. Reference may be had, e.g., to U.S. Pat. No. 3,597,679 (device for measuring magnetic field strength using a hall probe), U.S. Pat. No. 3,665,366 (Hall probe for measuring an axial magnetic field in a bore), U.S. Pat. No. 5,528,139 (Magnetic position sensor with hall probe in an air gap), U.S. Pat. No. 5,789,917 (magnetic position sensor with hall probe formed in an air gap of a stator), U.S. Pat. No. 6,043,645 (Magnetic position and speed sensor having a hall probe).U.S. Pat. No. 6,486,654 (calibration of magnetic force or scanning hall probe microscopes), U.S. Pat. No. 6,573,709 (position sensor with hall probe), and the like. The entire disclosure of each of these United States patents is hereby incorporated by reference into this specification.

One may also use a gaussmeter to measure the magnetic field strength at various positions within the lumen 10104. As is known to those skilled in the art, a gaussmeter is a magnetometer whose scale is graduated in gauss or kilogauss and which usually measures only the intensity and not the direction of the magnetic field. Reference may be had, e.g., to U.S. Pat. No. 4,063,158 (gaussmeter), U.S. Pat. No. 5,070,214 (organic material with extremely narrow electron spin resonance line and gaussmeter probe or magnetometer using this material), and the like. The entire disclosure of each of these United States patents is hereby incorporated by reference into this specification.

In one preferred embodiment, one can take measurements of points separated by one millimeter in a three-dimensional matrix to establish the alternating current magnetic flux local density. Thus, e.g., in a volume of 1,000 cubic millimeters (10 mm.×10 mm×10 mm), one could take at least 10 measurements along the X axis, and for each of these take 10 measurements along the Y axis, and for each of these take 10 measurements along the Z axis. The measurements thus taken could be used to calculate an average alternating current magnetic flux local density. At least about 95 weight percent of the points so measured would be within about plus or minus ten percent of the average alternating current magnetic flux local density; when this condition occurs, then it can be said that the magnetic field strength with the space being measured is substantially uniform.

The a.c. magnetic flux local density can be measured for a particular frequency or frequency range. Thus, e.g., the probes commonly used often have an adjustable band pass filter which allows you to measure the a.c. flux local density that corresponds to a electromagnetic radiation with a certain frequency or range of frequencies. Devices with such adjustable band pass filters are well known. Reference may be had, e.g., to U.S. Pat. Nos. 3,884,162; 3,945,008; 4,051,841; 4,083,031; 4,644,272; 5,843,133; 6,063,043; and the like. The entire disclosure of each of these United States patents is hereby incorporated by reference into this specification.

In one embodiment, and referring to FIG. 50, the radio frequency energy disposed within lumen 10104 is limited to a certain range of frequencies clustered around the “center frequency” of the MRI radiation used. In this embodiment, the MRI “center frequency” will be either 32 megahertz, 64 megahertz, 128 megahertz, or 256 megahertz; and the range of frequencies around such “center frequency” will be plus or minus 20 percent. Thus, if the “center frequency” is 32 megahertz, the range of frequencies will extend from 25.6 megahertz to 38.4 megahertz with a bandwidth of plus or minus 20 percent. Thus, if the “center frequency” is 64 megahertz, the range of frequencies will extend from 51.2 megahertz to 76.8 megahertz with a bandwidth of plus or minus 20%.

In one embodiment, the bandwidth over which the range of frequencies extends from the “center frequency” is plus or minus 15 percent and, more preferably, plus or minus 10 percent. In another embodiment, the bandwidth over which the range of frequencies extends from the center frequency is plus or minus 5 percent and, more preferably, plus or minus 1 percent.

In this embodiment, and referring again to FIG. 50, frequencies outside of the bandwidth are substantially excluded from the lumen 10104, and less than about 20 percent of the radiofrequency radiation within the lumen 10104 has a frequency outside of the bandwidth. Thus, e.g., where the center frequency is 64 megahertz, and the bandwidth extends from plus or minus 5 percent (from 60.8 megahertz to 67.2 megahertz), less than about 20 percent of the radiation within the lumen 10104 has a frequency below 60.8 megahertz and above 67.2 megahertz.

In one aspect of this embodiment, less than about 10 percent of the radiation within the lumen 10104 has a frequency outside of frequencies plus or minus 20 percent of the “center frequency.” In another aspect of this embodiment, less than about 5 percent of the radiation within the lumen 10104 has a frequency outside of frequencies plus or minus 20 percent of the “center frequency.” In yet another embodiment, less than about 1 percent of the radiation within the lumen 10104 has a frequency outside of frequencies plus or minus 20 percent of the “center frequency.”

In another embodiment, one may determine, by means discussed elsewhere in this specification, the average frequency within the lumen 10104 (see FIG. 50). In one aspect of this embodiment, It is preferred that the a.c. magnetic flux local density within the lumen 10104 be within plus or minus 10 percent of the average, and, more preferably, be within plus or minus 5 percent of the average.

FIG. 50 is a schematic illustration of a response of stent 10100 that is coated with nanomagnetic material (not shown) in accordance with the procedure of this invention; this stent is comprised of a lumen 10104.

Referring to FIG. 50, it will be seen that the magnetic lines of force 10102 are not distorted as much by applicants' stent 10100 as the lines of force 10002 are distorted by the stent 10000 of FIG. 49. Without wishing to be bound to any particular theory, applicants believe that, in one embodiment of their stent 101000, the “resonance circuits” formed are “local” rather than “global,” i.e., many different such “resonance circuits” are formed by many different combinations of nanomagnetic particles and dielectric matrix material.

In the embodiment depicted in FIG. 50, there is substantially no distortion caused by the “passive resonant circuits.” Thus, e.g., the field density at point 10007 is substantially identical to the field density at point 10005 (being within about 10 percent or less of the latter value). As is discussed elsewhere in this specification, at least about of the MRI electromagnetic radiation penetrates to the lumen 10104 of the device 10104; and the concentration of the electromagnetic radiation that penetrates to the lumen of the device is substantially identical at different points within such lumen.

If one were to assume that the stent 10100 were to be exposure to MRI electromagnetic radiation of, e.g., 64 megahertz, and if one also were to assume that objects 10006, 10008, and 10010 were not disposed within lumen 10104 during such exposure, then the field strength of the radiation within lumen 10104 would not only be at least about 90 percent of the field strength of the radiation outside of stent, but the field strength of the radiation at different points within the lumen 10104 would be substantially equal, being within about plus or minus 10 percent. Thus; e.g., in such a situation, where no material 10006/10008/10010 is disposed within the lumen 10104, the field strength at points 10009, 10011, 10013, 10015, 10017, 10019, 10021, and 10023 would be substantially equal.

Without wishing to be bound to any particular theory, applicants believe that the images 10107, 10109, and 10111 obtained with their stent 10100 provide a substantially greater degree of imaging resolution than does the image 10016 (see FIG. 49). The imaging resolution 10112 obtainable with applicants' process is at least 10 millimeters and, preferably, at least 5 millimeters. In one aspect of this embodiment, resolutions 10112 of at least one millimeter are often obtained.

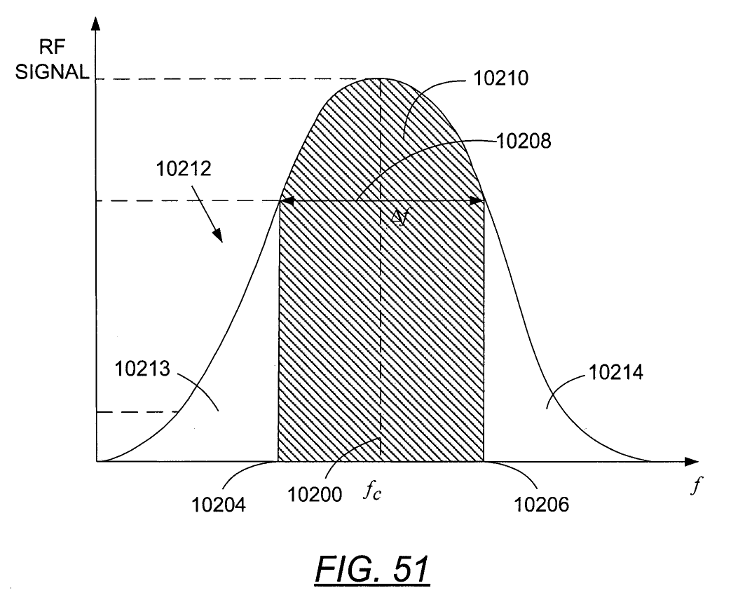

Referring again to FIG. 50, and in the preferred embodiment depicted therein, it will be seen that the stent assembly 10100 is comprised of a coating 10101 that preferably comprises nanomagnetic material. This coating 10101, and the stent assembly 10100, preferably have a bandwidth of less than about 20 percent at a center frequency of either 32 megahertz, 64 megahertz, 128 megahertz, or 256 megahertz, as is best illustrated in FIG. 51.

As is known to those skilled in the art, bandwidth is the difference between the frequency limits of a band containing the useful frequency limits of a signal. Reference may be had, e.g., to U.S. Pat. No. 3,622,919 (step attenuator of low inductance and high bandwidth), U.S. Pat. No. 4,615,034 (ultra-narrow bandwidth optical thin film interference coatings), U.S. Pat. No. 4,731,881 (narrow spectral bandwidth UV solar blind detector), U.S. Pat. No. 5,189,532 (edge-illuminated narrow bandwidth holographic filter), U.S. Pat. No. 5,486,935 (high efficiency chiral nematic liquid crystal rear polarizer for liquid crystal displays having a notch polarization bandwidth of 100 nanometers to 250 nanometers), U.S. Pat. No. 5,770,304 (wide bandwidth electromagnetic wave absorbing material), U.S. Pat. No. 6,854,986 (very high bandwidth electrical interconnect), and the like. The entire disclosure of each of these United States patents is hereby incorporated by reference into this specification.

Referring to FIG. 51, it is preferred that the coated stent assembly 10100 (see FIG. 50) pass a range of frequencies about its center frequency 10200 (see FIG. 51), and between frequencies 10206 and 10204, such that the bandwidth 10208/2 is no greater than plus or minus about 20 percent of the center frequency 10200. In one embodiment, the bandwidth 10208/2 is no greater than plus or minus about 15 percent of the center frequency 10200.

Referring again to FIG. 51, it is preferred that the center frequency 10200, “f_{c}” be either 32 megahertz, 64 megahertz, 128 megahertz, or 256 megahertz. When the center frequency 10200 “f_{c}” is, e.g., 64 megahertz and the bandwidth 10208 is plus or minus 10 percent, the bandwidth 10208 extends from a frequency 10204 of 57.6 mega to a frequency 10206 about 70.4 megahertz; and, in this case, the bandwidth 10208 is 12.8 megahertz.

Referring again to FIG. 51, and to the preferred embodiment depicted therein, only the shaded area 10210 of the radiofrequency signal 10212 will preferably pass substantially unattenuated from the exterior of stent assembly 10100 (see FIG. 50) into the lumen 10104 of the stent 10100.

At radio frequencies below the limits of the bandwidth 10208 (see area 10213), and/or above the limits of the bandwidth 10208 (see area 10214), less than 90 percent of the “unshaded portions” of radio frequency signal 10212 will pass substantially unattenuated form the exterior of the stent assembly 101000 into the lumen 10104 of the stent 10100. The degree of attenuation of the radio frequency signal may be measured by determining the amplitude of the signal, or its field strength.

In one embodiment, less than about 50 percent of the radio frequency signal will pass substantially unattenuated from the exterior of the stent assembly 101000 into the lumen 10104 of the stent 10100 when that signal has a frequency above frequency 10206 or below frequency 10204. In another embodiment, less than about 20 percent of the radio frequency signal will pass substantially unattenuated form the exterior of the stent assembly 101000 into the lumen 10104 of the stent 10100 when that signal has a frequency above frequency 10206 or below frequency 10204. In another embodiment, less than about 10 percent of the radio frequency signal will pass substantially unattenuated form the exterior of the stent assembly 101000 into the lumen 10104 of the stent 10100 when that signal has a frequency above frequency 10206 or below frequency 10204. In another embodiment, less than about 5 percent of the radio frequency signal will pass substantially unattenuated form the exterior of the stent assembly 101000 into the lumen 10104 of the stent 10100 when that signal has a frequency above frequency 10206 or below frequency 10204.

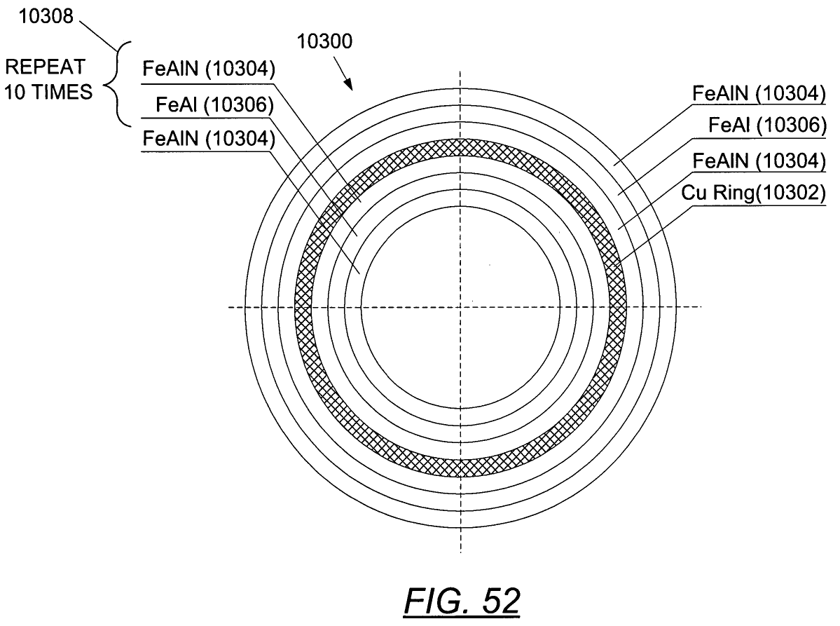

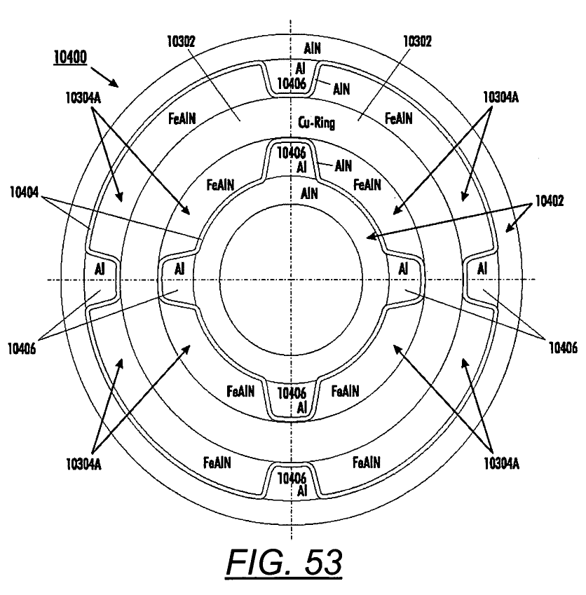

FIG. 52 is a schematic of a coated substrate 10300 comprised of a substrate 10302 that, in one preferred embodiment, is a copper ring with a thickness of 0.030.” In another embodiment, not shown, the substrate 10302 is a metallic stent that may consist of or comprise Nitinol.

Referring to FIG. 52, and to the preferred embodiment depicted, the copper ring 10302 is coated on its top and bottom surfaces with a layer 10304 of FeAlN that has a thickness of 1,000 angstroms. Contiguous with the layer 10304 of FeAlN is a layer 10306 of FeAl that has a thickness of 100 angstroms.

In one embodiment, the combination 10308 of the FeAlN 10304 layer/FeAl 10306 layer can be repeated symmetrically or asymmetrically on the top and the bottom surfaces of the substrate 10302.

FIG. 53 is a schematic of a coated substrate 10400 that is similar to the coated substrate 10300 but differs therefrom n that the layers 10304A of FeAlN has a thickness of 10,000 angstroms, the coating is comprised of layers 10402 of AlN with a thickness of 5,000 angstroms, the coating is comprised of thinner layers 10404 of AlN with a thickness of 2,000 angstroms, and the coated is also comprised of filled aluminum vias with a thickness of 10,000 angstroms.

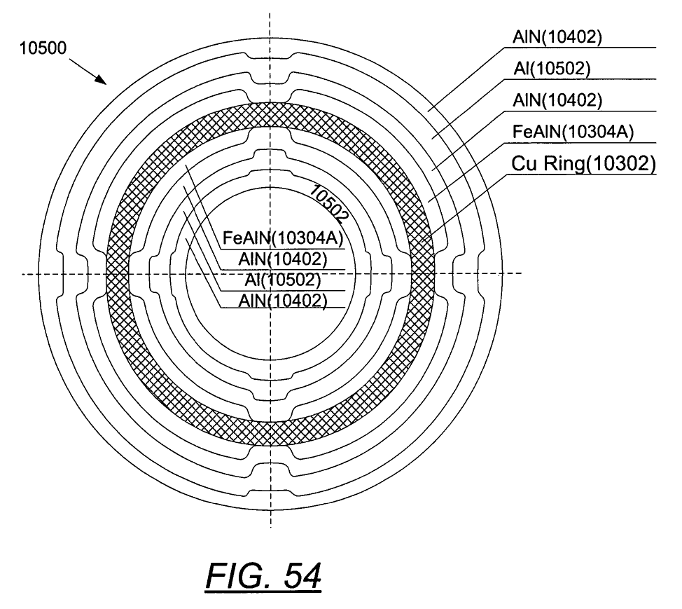

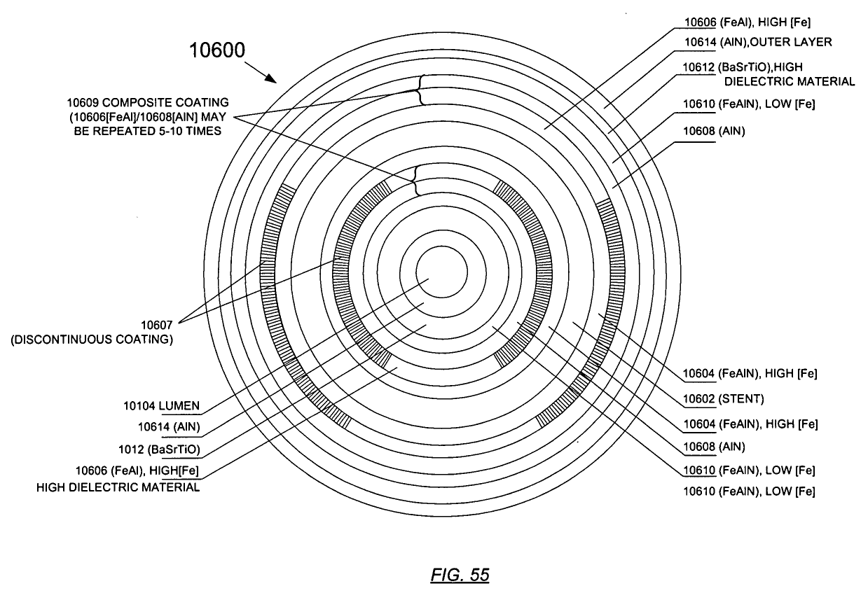

FIG. 54 is a schematic of a coated substrate 10500 that is similar to coated substrates 10300 and 10400 but that also comprises layers 10502 of aluminum with a thickness of 5,000 angstroms.

FIG. 55 is a schematic of coated stent assembly 10600 that is preferably comprised of one or more of the metallic stents 10602 described elsewhere in this specification. In one aspect of this embodiment, the Nitinol stent has a diameter of about 6 millimeters.

Referring again to FIG. 55, and to the preferred embodiment depicted therein, disposed above and below the stent 10602, and contiguous therewith, is a layer 10604 of FeAlN that preferably contains more than 60 mole percent of Fe, by combined moles of Fe and Al. In one preferred embodiment, layer FeAlN contains 82.5 weight percent of Fe by combined weight of Fe and Al. It is preferred that layer 10604 be relatively thin, ranging from about 100 to about 1000 angstroms. In one embodiment, layer 10604 is about 500 angstroms thick.

Referring again to FIG. 55, and to the preferred embodiment depicted therein, disposed above and below 10604, and contiguous therewith, is a layer 10606 of FeAl that preferably contains more than 60 mole percent of Fe, by combined moles of Fe and Al. In one preferred embodiment, layer FeAl contains 82.5 weight percent of Fe by combined weight of Fe and Al. It is preferred that layer 10606 be relatively thin, preferably being less than about 500 angstroms thick.

In one preferred embodiment, illustrated in FIG. 55, the FeAl coating is discontinuous, i.e., it does not necessarily extend continuously around the periphery of the 10604 coating and may have one or more discontinuities, i.e., areas where the FeAl coating does not appear. The discontinuities 10607 are illustrated in FIG. 55 merely for purposes of illustration, it being apparent that such discontinuities may appear at other portions of the FeAl coating and/or, in one embodiment, not at all.

Referring again to FIG. 55, and to the preferred embodiment depicted therein, disposed above and below the layer 10606, and contiguous therewith, is a layer 10608 of AlN that has a thickness of from about 100 to about 1,000 angstroms and, in one embodiment, has a thickness of about 500 angstroms. The layers 10606/10608 form a composite FeAl/AlN coating 10609 that may be repeated for from, e.g., 5 to 10 times. In one embodiment, the composite FeAl/AlN coating 10609 has a total thickness of from about 300 to about 800 nanometers and, more preferably from about 450 to about 550 nanometers.

Referring again to FIG. 55, and to the preferred embodiment depicted therein, disposed above and below the ;layer 0608, and contiguous therewith, is a layer 10610 of FeAlN that preferably contains relatively low amounts of Fe. In one preferred embodiment, layer 10610 contains less than 15 weight percent of Fe by combined weight of Fe and Al and, more preferably, less than 11 weight percent of Fe by combined weight of Fe and Al. In one embodiment, layer, layer 10610 contains from about 5 to about 11 weight percent of Fe, by combined weight of Fe and Al. It is preferred that layer 10610 be relatively thin, ranging from about 100 to about 500 angstroms.

Referring again to FIG. 55, and to the preferred embodiment depicted therein, disposed above and below the lawyer 10610, and contiguous therewith, is a layer 10612 of a material with a high dielectric constant of at least about 80 and preferably at least about 100. One may use any of the high dielectric materials described elsewhere in this specification such as, e.g., barium strontium titanate. The layer 10612 preferably has a thickness of from about 500 to about 5000 angstroms; in one embodiment, layer 10612 has a thickness of about 3000 angstroms.

Referring again to FIG. 55, and to the preferred embodiment depicted therein, disposed above and below the stent 10612, and contiguous therewith, is an outer layer 10614 of AlN that preferably has a thickness of from about 100 to about 500 angstroms and, in one embodiment, has a thickness of about 300 angstroms.

Although the invention has been described herein with respect to certain preferred embodiments, numerous modifications and alterations may be made to the described embodiment without departing from the spirit and intended scope of the invention. It is intended to include any and all such modifications and alterations within the scope of the following claims and/or the equivalents thereof.

1. A medical device comprised of a lumen wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen.

2. A coated medical device assembly comprised of a medical device, a coating disposed on said medical device, and a cavity disposed within said medical device, wherein:
(a) said coating is comprised of material with a dielectric constant of from about 1 to about 2,000, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

3. The medical device assembly as recited in claim 2, wherein said medical device is a stent.

4. A medical device with an inductance of from about 0.1 to about 5 nanohenries and a capacitance of from about 0.1 to about 10 nanofarads, wherein said device is comprised of an exterior wall and an interior cavity, and wherein, when said device is exposed to radio frequency electromagnetic radiation with a frequency of from 10 megahertz to about 200 megahertz, at least 90 percent of the electromagnetic radiation penetrates to the cavity of the device; and the concentration of the electromagnetic radiation that penetrates to the cavity of the device is substantially identical at different points within such cavity.

5. A coated medical device assembly comprised of a medical device, a coating disposed on said medical device, and a cavity disposed within said medical device, wherein:
(a) said coating is comprised of material with a conductivity of from about 10⁻¹³ (ohm-meter)⁻¹ to about 10⁸ (ohm-meter)⁻¹, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

6. The coated medical assembly as recited in claim 5, wherein said coating is comprised of material with a conductivity of and from about 10⁻³ (ohm-meter)^{31 1} to about 10 (ohm-meter)⁻¹.

7. A coated assembly with an inductance of from about 0.1 to about 5 nanohenries and a capacitance of from about 0.1 to about 10 nanofarads, wherein said coated assembly is comprised of a substrate and a coating disposed thereon, wherein said coating is comprised of magnetic particles with a particle size in the range of from about 3 to about 20 nanometers, wherein said coating has a top surface and a bottom surface, wherein said bottom surface is contiguous with said substrate, and wherein at least 1.5 times as many of said magnetic particles are disposed near said bottom surface of said stent than near said top surface of said stent.

8. A coated assembly with an inductance of from about 0.1 to about 5 nanohenries and a capacitance of from about 0.1 to about 10 nanofarads, wherein said coated assembly is comprised of a stent and a coating disposed thereon, wherein said coated stent assembly is comprised of a lumen, and biological material disposed within said lumen, and wherein, when said stent is exposed to radio frequency electromagnetic radiation with a frequency of from 10 megahertz to about 200 megahertz, said coated stent assembly has a radio frequency shielding factor of less than about 10 percent, at least 90 percent of said electromagnetic radiation penetrating said stent and contacting said biological material disposed within said lumen.

9. The coated stent assembly as recited in claim 8, wherein said stent has a substantially constant radio frequency shielding factor along the length of said stent.

10. A coated assembly with an inductance of from about 0.1 to about 5 nanohenries and a capacitance of from about 0.1 to about 10 nanofarads, wherein said assembly is comprised of a coating, and wherein said coating that has a relative permeability of at least 1.1 over the range of frequencies of from about 10 megahertz to about 200 megahertz, an increase of such relative permeability over such range of from about 1×10⁻¹⁴ to about 1×10⁻⁶ per hertz, and a magnetization, when measured at a direct current magnetic field of 2 Tesla, of from about 0.1 to about 10 electromagnetic units per cubic centimeter.

11. The coated assembly as recited in claim 4, wherein said coated assembly further comprises a substrate on which said coating is disposed.

12. The coated assembly as recited in claim 5, wherein said substrate is a stent.

13. The coated assembly as recited in claim 2, wherein said coating is comprised of particles of nanomagnetic material.

14. The coated assembly as recited in claim 13, wherein said particles of said nanomagnetic material are at least triatomic, being comprised of a first distinct atom, a second distinct atom, and a third distinct atom.

15. The coated assembly as recited in claim 14, wherein said first distinct atom is an atom selected from the group consisting of atoms of actinium, americium, berkelium, californium, cerium, chromium, cobalt, curium, dysprosium, einsteinium, erbium, europium, fermium, gadolinium, holmium, iron, lanthanum, lawrencium, lutetium, manganese, mendelevium, nickel, neodymium, neptunium, nobelium, plutonium, praseodymium, promethium, protactinium, samarium, terbium, thorium, thulium, uranium, and ytterbium, and mixtures thereof.

16. The coated assembly as recited in claim 15, wherein said second distinct atom is selected from the group consisting of silicon, aluminum, boron, platinum, tantalum, palladium, yttrium, zirconium, titanium, calcium, cerium, beryllium, barium, silver, gold, indium, lead, tin, antimony, germanium, gallium, tungsten, bismuth, strontium, magnesium, zinc, and mixtures thereof.

17. The coated assembly as recited in claim 16, wherein from about 2 to about 20 mole percent of said first distinct atom is present in said coating, by combined moles of said first distinct atom and said second distinct atom.

18. The coated assembly as recited in claim 16, wherein from about 5 to about 10 mole percent of said first distinct atom is present in said coating, by combined moles of said first distinct atom and said second distinct atom.

19. The coated assembly as recited in claim 10, wherein from about 6 to about 8 mole percent of said first distinct atom is present in said coating.

20. The coated assembly as recited in claim 10, wherein said first distinct atom is iron and said second distinct atom is aluminum.

21. A coated medical device assembly comprised of a medical device, a coating disposed on said medical device, and a cavity disposed within said medical device, wherein:
(a) said coating is comprised of material with a magnetization when measured at a direct current magnetic field of 2 Tesla of from about 0.2 to about 1 electromagnetic units per cubic centimeter, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

22. The coated medical device assembly as recited in claim 21, wherein said medical device is a stent.

23. The coated assembly as recited in claim 22, wherein said coating is comprised of material with a magnetization when measured at a direct current magnetic field of 2 Tesla of from about 0.2 to about 0.8 electromagnetic units per cubic centimeter

24. A coated medical device assembly comprised of a medical device, a coating disposed on said medical device, and a cavity disposed within said medical device, wherein:
(a) said coating has a relative magnetic permeability when measured at a radio frequency of 64 megahertz of at least 1.2, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

25. The coated assembly as recited in claim 24, wherein said coating has a relative permeability when measured at a radio frequency of 64 megahertz of at least 1.3.

26. The coated assembly as recited in claim 13, wherein said particles of nanomagnetic material are comprised of a said first distinct atom, said second distinct atom, said third distinct atom, and a fourth distinct atom.

27. The coated assembly as recited in claim 13, wherein said particles of nanomagnetic material have a squareness is from about 0.2 to about 0.8.

28. The coated assembly as recited in claim 13, wherein said particles of nanomagnetic material have an average size of less of less than about 50 nanometers.

29. The coated assembly as recited in claim 13, wherein said particles of nanomagnetic material have an average size of less of less than about 20 nanometers.

30. The coated assembly as recited in claim 13, wherein said particles of nanomagnetic material have a phase transition temperature of less than about 50 degrees Celsius.

31. The coated assembly as recited in claim 13, wherein said particles of nanomagnetic material have a saturation magnetization of at least about 1,000 electromagnetic units per cubic centimeter.

32. A coated medical device assembly comprised of a medical device, a coating disposed on said medical device, and a cavity disposed within said medical device, wherein said coated assembly has a magnetic susceptibility within the range of plus or minus 1×10⁻³ centimeter-gram-seconds, and wherein, when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

33. The coated assembly as recited in claim 13, wherein the average coherence length between adjacent nanomagnetic particles is less than 100 nanometers

34. The coated assembly as recited in claim 33, wherein said nanomagnetic material has a saturation magnetization of at least 2,000 electromagnetic units per cubic centimeter.

35. The coated assembly as recited in claim 13, wherein said particles of nanomagnetic material are disposed within an insulating matrix.

36. The coated assembly as recited in claim 35, wherein said coating has a thickness of from about 400 to about 2000 nanometers.

37. A coated medical device assembly comprised of a medical device, a coating disposed on said medical device, and a cavity disposed within said medical device, wherein:
(a) said coating has a morphological density of at least about 99 percent, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

38. A coated medical device assembly comprised of a medical device, a coating disposed on said medical device, and a cavity disposed within said medical device, wherein:
(a) said coating has an average surface roughness of less than about 10 nanometers, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

39. A coated medical device assembly comprised of a medical device, a coating disposed on said medical device, and a cavity disposed within said medical device, wherein:
(a) said coating is biocompatible, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

40. A coated medical device assembly comprised of a medical device, a coating disposed on said medical device, and a cavity disposed within said medical device, wherein:
(a) said coating is hydrophohtic, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

41. A coated medical device assembly comprised of a medical device, a coating disposed on said medical device, and a cavity disposed within said medical device, wherein:
(a) said coating is hydrophilic, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.

42. A medical device comprised of superparamagnetic material and a lumen wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen.

43. A medical device comprised of material with a saturation magnetization of at least 2,500 electromagnetic units per cubic centimeter and a lumen wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen.

44. A medical device comprised of material with a coercive force of from about 0.1 to about 10 Oersteds and a lumen wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen.

45. A medical device comprised of antithrombogenic material and a lumen wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen.

46. A medical device with a bend radius of at least 2 centimeters, wherein said medical device is comprised of a lumen, and wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen.

47. A medical device comprised of nanoelectrical material with a resistivity of from about 1 to about 100, wherein said medical device is comprised of a lumen, and wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen. microohm-centimeters.

48. A medical device with a relative dielectric constant of from about 1 to about 100, wherein said medical device is comprised of a lumen, and wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen.

49. A medical device comprised of nanoconductive material with a resistivity at 20 degrees Centigrade of from about 1×10⁻⁶ ohm-centimeters to about 1×10⁻⁵ ohm-centimeters and an average particle size of less than about 100 nanometers, wherein said medical device is comprised of a lumen, and wherein, when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen.

50. A medical device with a relative dielectric constant of from about 1 to about 100, wherein:
(a) said medical device is comprised of a lumen,
(b) when said device is exposed to radio frequency electromagnetic radiation with a frequency of from 10 megahertz to about 200 megahertz, at least 90 percent of the electromagnetic radiation penetrates to the lumen of the device; and the concentration of the electromagnetic radiation that penetrates to the lumen of the device is substantially identical at different points within such lumen, and
(c) the product of said relative dielectric constant of said medical device and the relative magnetic permeability of such medical device is at least 10.

51. The medical device as recited in claim 50, wherein said product of said relative dielectric constant of said medical device and the relative magnetic permeability of such medical device is at least 100; and wherein said relative magnetic permeability of such medical device is at least 1.

52. The medical device as recited in claim 51, wherein said product of said relative dielectric constant of said medical device and the relative magnetic permeability of such medical device is at least 1,000.

53. The medical device as recited in claim 51, wherein said product of said relative dielectric constant of said medical device and the relative magnetic permeability of such medical device is at least 5,000.

54. The medical device as recited in claim 51, wherein said product of said relative dielectric constant of said medical device and the relative magnetic permeability of such medical device is at least 10,000.

55. A coated medical device assembly comprised of a medical device, a coating disposed on said medical device, and a cavity disposed within said medical device, wherein:
(a) said medical device has a resistivity at 20 degrees Centigrade of from about 1 to about 100 microohm-centimeters, and
(b) when said medical device is exposed to radio frequency electromagnetic radiation with a frequency of from 10 megahertz to about 200 megahertz, at least 90 percent of the electromagnetic radiation penetrates to the cavity of said medical device; and the concentration of the electromagnetic radiation that penetrates to said cavity of said device is substantially identical at different points within said cavity.
